(19) United States
(12) Patent Application Publication
(10) Pub. No.: US 2025/0208123 A1
(43) Pub. Date: Jun. 26, 2025

(54) Multi-Omic Integration Platform

(71) Applicant: Early is Good, Inc., Indianapolis, IN (US)

(72) Inventors: Thakshila Udayakanthi Habarakada Liyanage, Indianapolis, IN (US); Asel Ananda Habarakada Liyanage, Indianapolis, IN (US); Ananda Piyankara Habarakada Liyanage, Indianapolis, IN (US)

(21) Appl. No.: 18/988,772

(22) Filed: Dec. 19, 2024

Related U.S. Application Data
(60) Provisional application No. 63/704,465, filed on Oct. 7, 2024.
(60) Provisional application No. 63/575,570, filed on Apr. 5, 2024.
(60) Provisional application No. 63/613,004, filed on Dec. 20, 2023.

(51) Int. Cl. G01N 33/542; G01N 33/53; G01N 33/543; G01N 33/553; G01N 33/68

(52) U.S. Cl. CPC G01N 33/542; G01N 33/5308; G01N 33/54346; G01N 33/553; G01N 33/6803

(57) ABSTRACT

The present invention discloses a system and method for detecting a target analyte using multiple distinct probes, such as a capture probe, a donor probe, and an acceptor probe. The method involves the specific binding of a capture probe to a target analyte, followed by the hybridization of a donor probe and the acceptor probe to adjacent regions of the target analyte. Upon excitation, the donor probe transfers energy to the acceptor probe, resulting in a detectable signal. The use of these probes allows for highly sensitive and specific detection of nucleic acids, proteins and small molecules.

### CROSS-REFERENCE TO RELATED APPLICATIONS

This application claims benefit of priority under 35 U.S.C. § 119(e) of U.S. Ser. No. 63/613,004, filed Dec. 20, 2023; U.S. Ser. No. 63/575,570, filed Apr. 5, 2024; and U.S. Ser. No. 63/704,465, filed Oct. 7, 2024, the contents of which are incorporated herein by reference in their entirety.

### BACKGROUND INFORMATION

### Field of the Invention

The present disclosure relates generally to a system for detecting nucleic acids, proteins and small molecules, and more specifically to methods of detection using high sensitivity molecular marker detection.

### Background Information

Various methods have been developed for detecting molecular analytes such as DNA, RNA, and protein. Such detection methods play a crucial role in disease analysis, diagnostics, and research. One common approach involves the use of probes that specifically bind to the target analytes of interest. These probes can be labeled with fluorescent or other detectable markers to enable visualization and quantification of the target nucleic acid.

In some existing methods, a single probe is used to detect a target nucleic acid or protein. This probe, known as a capture probe, is designed to bind to a specific region of the target analyte. Upon binding, the capture probe can be detected using various techniques, such as fluorescence or colorimetric assays. While this approach has been widely used, it suffers from limitations in terms of specificity and sensitivity, as a single probe may not provide sufficient discrimination against non-specific binding or background noise.

Another approach involves the use of two probes, known as a donor probe and an acceptor probe, which are designed to hybridize to adjacent regions of the target analyte. The donor probe is labeled with a donor fluorophore, while the acceptor probe is labeled with an acceptor fluorophore. The donor fluorophore is compatible with the acceptor fluorophore, and following excitation of the donor fluorophore, energy is transferred from the donor fluorophore to the acceptor fluorophore, causing detectable emission of energy from the fluorophores. When the two probes hybridize to the target analyte, the proximity of the donor and acceptor fluorophores allows for energy transfer, resulting in a detectable signal. This method, known as fluorescence resonance energy transfer (FRET), can provide enhanced specificity and sensitivity compared to single-probe methods. However, it may still be limited by the need for careful design and optimization of the probe sequences and fluorophores.

Other approaches have utilized more than two probes to detect nucleic acids and proteins, such as three or more probes. These multi-probe methods aim to further improve specificity and sensitivity by incorporating additional probes that can bind to different regions of the target nucleic acid sequence. However, these approaches have often been complex and challenging to implement, requiring careful probe design, optimization, and analysis.

### SUMMARY OF THE INVENTION

The present disclosure provides a surprisingly multifunctional detection system that detects several analytes simultaneously with high sensitivity, accuracy, and specificity.

In one embodiment, the present disclosure is directed to a target analyte detection system including: (a) a capture probe including an affinity tag and a cleavable linker or photo-cleavable spacer; (b) a donor probe; (c) an acceptor probe; (d) a nanoparticle array including a substrate with nanoparticles, functionalized with the acceptor probes on the surface of the substrate and a spacer between the nanoparticle and the acceptor probe; and (e) a universal signal enhancer solution comprising nanoparticles, wherein each nanoparticle is conjugated with one or more of: an anti-bioluminescent antibody for donor bioluminescent tag, an anti-fluorescent antibody for donor fluorescent tag, an anti-chemiluminescent antibody for donor chemiluminescent tag and an affinity tag, wherein the donor probe and the acceptor probe comprise a BRET signal pair, a FRET signal pair, a bioluminescent subunit complex, a bioluminescent signal emitter, a fluorescent signal emitter, a chemiluminescent signal emitter, a LSPR signal, or an LSPR signal pair.

In some aspects, the donor probe includes an affinity tag and a bioluminescent tag, a first fluorophore tag, first bioluminescent subunit tag or a chemiluminescence tag.

In some aspects, the acceptor probe includes an affinity tag and one or more of: a fluorescence tag compatible with the bioluminescent tag, a second fluorophore tag compatible with the first fluorophore tag, or a second bioluminescent subunit tag compatible with the first bioluminescent subunit tag.

In some aspects, the cleavable linker or spacer includes a cleavable chemical group that can be selectively broken under predetermined conditions.

In some aspects, the detection mechanism includes one or more resonance energy transfer (RET) mechanisms, including but not limited to BRET, FRET, and similar processes, for detecting and measuring interactions between donor and acceptor molecules. In some aspects, the detection mechanism includes one or more of: BRET, FRET, CRET, ECL, SPA, QRET, LSPR, BiFC, TR-FRET, fluorescence anisotropy, SPR, luminescence, chemiluminescence, Raman scattering, or FLIM.

In some aspects, the BRET signal pair, the FRET signal pair, the bioluminescent signal pair, or the fluorescence signal pair is located between 5 to 60 nm from nanoparticles in the nanoparticle array. In some aspects, the BRET signal pair, the FRET signal pair, the bioluminescent signal pair, or the fluorescence signal pair is located between 5 to 60 nm from the nanoparticle of the universal signal enhancer solution.

In some aspects, signal enhancement is achieved using Localized Surface Plasmon Resonance (LSPR) of the nanoparticle of the nanoparticle array and the nanoparticle of the universal signal enhancer solution. In some aspects, signal enhancement is achieved using LSPR of the nanoparticle of the nanoparticle array only. In some aspects, signal enhancement is achieved using LSPR of the nanoparticle of the universal signal enhancer solution only.

In some aspects, analyte detection can be used without signal enhancement from the nanoparticle of the nanoparticle array or the nanoparticle of the universal signal enhancer solution.

In some aspects, the target analyte is a nucleic acid or a protein.

In some aspects, the nucleic acid is DNA, lncRNA, mRNA, tRNA, rRNA, or small RNA.

In some aspects, the small RNA is selected from the group consisting of short RNA, microRNA (miRNA), tiny non-coding RNAs (tncRNA), small modulatory RNA, small interfering RNAs (siRNAs), Piwi-interacting RNAs (piRNAs), transfer RNA-derived small RNAs (tsRNAs or tRFs), small nuclear RNAs (snRNAs), small nucleolar RNAs (snoRNAs), small RNA fragments derived from ribosomal RNAs (rRFs), small modulatory RNAs (smRNAs), small guide RNAs (sgRNAs), and small temporal RNAs (stRNAs).

In some aspects, the target analyte detection system further includes an extended probe.

In some aspects, the extended probe includes a sequence of 25-100 nucleotides, wherein about 6-20 nucleotides are complementary to a portion of the small RNA, and remaining bases of the extended probe are complementary to one or more of the donor probe and the acceptor probe.

In some aspects, the capture probe includes a nucleotide sequence from about 10 to 100 or 25 to 50 nucleotides in length. In some aspects, the capture probe is complementary to a target analyte. In some aspects, the capture probe includes an affinity tag and a cleavable spacer attached to the 5′ end of the capture probe.

In some aspects, for detection of small RNA, the target analyte detection system further includes: an extended probe, wherein: the extended probe is configured to hybridize with a portion of the small RNA; the capture probe is configured to hybridize with a portion of the small RNA distinct from the portion hybridized by the extended probe; and the extended probe is configured to hybridize with the donor probe and the acceptor probe.

In some aspects, for detection of mRNA, lncRNA, or DNA: the donor probe and the acceptor probe are configured to hybridize with the target analyte; and the system does not include an extended probe.

In some aspects, the affinity tag includes His-tag, FLAG-tag, Glutathione S-transferase (GST) tag, Maltose Binding Protein (MBP) tag, Strep-tag, HA-tag, Myc-tag, Avi-tag, V5-tag, T7-tag, biotin or its derivatives, SNAP tag, CLIP tag, or HaloTag®. In some aspects, the affinity tag includes amine, carboxylic acid, amino acid, thiol (SH), hydroxyl (OH), phosphate, azide group, alkaline group, ketone group, biotin or a functional derivative thereof, or a halide group, or a functional derivative thereof. In some aspects, the affinity tag includes biotin or a functional derivative thereof.

In some aspects, the cleavable spacer is a photo-cleavable spacer.

In some aspects, the donor probe includes a nucleotide sequence of about 10-100 nucleotides or about 15 to 25 nucleotides in length. In some aspects, the donor probe is complementary to the target analyte or the extended probe. In some aspects, the donor probe includes a bioluminescent tag or a fluorophore tag or bioluminescent subunit tag or chemiluminescence tag attached to the 3′ end of the donor probe and an affinity tag attached to the 5′ end.

In some aspects, the bioluminescent tag is Luciferin, Luciferyl adenylate firefly luciferase, Renilla luciferase, aequorin, Gaussia luciferase, or bacterial luciferase, coelenterazine aequorin, dinoflagellate luciferin Photoprotein, nanoluc luciferase, cypridina luciferase, nanobit-smallbit, nanobit-largebit or a functional equivalent thereof.

In some aspects, the acceptor probe includes a nucleotide sequence from about 6 to 100 nucleotides or about 10 to 25 nucleotides in length. In some aspects, the acceptor probe is complementary to the target analyte or the extended probe. In some aspects, the acceptor probe includes an affinity tag and one or more of: a fluorescent tag compatible with the bioluminescent tag, a second fluorescent tag compatible with the first fluorescent tag, a second bioluminescent subunit tag compatible with the first bioluminescent subunit tag or a fluorescent tag compatible chemiluminescence tag. In some aspects, the acceptor probe includes a spacer between the affinity tag and the nucleotide sequence.

In some aspects, the spacer includes hydrocarbon chain, polyethylene glycol (PEG), a polyamino acid, a polyacrylamide, polyvinylpyrrolidone, a zwitterionic polymer, a polysaccharide, poly(N-(2-hydroxypropyl)methacrylamide), poly(oligo(ethylene glycol), methylether methacrylate, a carboxylic dextran, a hydrocarbon chain, a substituted hydrocarbon chain, a silane group, 3-mercaptopropyl triethoxysilane (MPTES), or a combination thereof.

In some aspects, the spacer includes a hydrocarbon chain. In some aspects, the spacer includes a 1-20 nm distance from nanoparticles on the nanoparticle array.

In some aspects, the acceptor probe is bound to the donor probe via analyte to create a bioluminescent resonance energy transfer (BRET) complex. In some aspects, the acceptor probe is bound to the donor probe via analyte to create a fluorescence resonance energy transfer (FRET) complex. In some aspects, the acceptor probe is bound to the donor probe via analyte to create a bioluminescent complex. In some aspects, the acceptor probe is bound to the donor probe via analyte to create a bioluminescent signal. In some aspects, the acceptor probe is bound to the donor probe via analyte to create a fluorescence signal. In some aspects, the acceptor probe is bound to the donor probe via analyte to create a chemiluminescence signal.

In some aspects, the target analyte is a protein, and wherein the capture probe is a capture antibody, the donor probe is a donor antibody, the acceptor probe is an acceptor antibody.

In some aspects, the capture antibody includes a photocleavable spacer with a biotin label, and epitope binding sites for the target analyte.

In some aspects, the detection system further includes an affinity tag to bind with the nanoparticle of the universal signal enhancer solution, wherein the affinity tag is located on either: (a) the capture antibody; or (b) the donor antibody.

In some aspects, the affinity tag includes: His-tag, FLAG-tag, Glutathione S-transferase (GST) tag, Maltose Binding Protein (MBP) tag, Strep-tag, HA-tag, Myc-tag, Avi-tag, V5-tag, T7-tag, biotin, SNAP tag, CLIP tag, HaloTag®, amine, carboxylic acid, amino acid, thiol (SH), hydroxyl (OH), phosphate, azide group, alkaline group, ketone group, or a halide group, or a functional equivalent thereof.

In some aspects, the donor antibody includes a bioluminescent tag and epitope binding sites for the target analyte.

In some aspects, the detection system further includes an affinity tag to bind with the nanoparticle of the universal signal enhancer solution, wherein the affinity tag is located on either: (a) the capture antibody; or (b) the donor antibody.

In some aspects, the bioluminescent tag is Luciferin, Luciferyl adenylate firefly luciferase, Renilla luciferase, aequorin, Gaussia luciferase, or bacterial luciferase, coelenterazine aequorin, dinoflagellate luciferin Photoprotein, nanoluc luciferase, cypridina luciferase, nanobit-smallbit, nanobit-largebit or a functional equivalent thereof. In some aspects, the bioluminescent tag is luciferin or a functional equivalent thereof.

In some aspects, the acceptor antibody includes a fluorescent tag or a bioluminescent tag, an affinity tag, and binding sites for the donor antibody, wherein the donor antibody is complexed with the target analyte and the capture antibody. In some aspects, the acceptor antibody is bound to the donor antibody to create a bioluminescent resonance energy transfer (BRET) complex. In some aspects, the acceptor antibody is bound to the donor antibody to create a fluorescence resonance energy transfer (FRET) complex. In some aspects, the acceptor antibody is bound to the donor antibody to create a bioluminescent complex. In some aspects, the acceptor antibody is bound to the donor antibody to create a bioluminescent signal. In some aspects, the acceptor antibody is bound to the donor antibody to create a fluorescence signal. In some aspects, the acceptor antibody is bound to the donor antibody to create a chemiluminescence signal. In some aspects, the acceptor antibody is bound to the donor antibody to create a LSPR signal.

In some aspects, the substrate includes a custom nanofiber array, glass, an elastomeric polymer, polydimethylsiloxane (PDMS), ECOFLEX®, SILBIONE®, polyethylene terephthalate (PET), polyurethane (PU), polyethylene naphthalate (PEN), a polyimide (PI), polybutadiene, polyisoprene, a silane, a polyamine, polymethylmethacrylate (PMMA), polydopamine, polyvinylpyrrolidone (PVP), polyvinyl alcohol (PVA), a polyolefin, a polyamide, a polyimide, a protein, silk, cellulose, a polyelectrolyte, a peptoid, or a combination thereof.

In some aspects, the nanoparticle includes gold, silver, iron oxide, a quantum dot, a carbon-based nanoparticle, a chemical nanoparticle, a liposome, a polymeric nanoparticle, a dendrimer, a magnetic nanoparticle, a silica nanoparticle, a metal oxide nanoparticle, a semiconductor nanoparticle, an MXene nanoparticle, or a combination thereof. In some aspects, the nanoparticle is spherical, rod-shaped, triangular, prismatic, cube-shaped, star-shaped, wire-shaped, a sheet, tube-shaped, hollow or cage-like, flower-shaped, disk-shaped, a nanopyramid, a nanobipyramid, a nanoplate, a self-assembled nanostructure, a bowtie antenna, a nano island, a nanoshell, or a combination thereof.

In some aspects, the nanoparticle is about 4 nm to 2000 nm.

In some aspects, the spacer between the nanoparticle and the acceptor probe has a thickness from about 0.5 nm to 20 nm. In some aspects, the spacer between the nanoparticle and the acceptor antibody has a thickness from about 0.5 nm to 20 nm. In some aspects, the spacer includes polyethylene glycol (PEG), a polyamino acid, a polyacrylamide, polyvinylpyrrolidone, a zwitterionic polymer, a polysaccharide, poly(N-(2-hydroxypropyl)methacrylamide), poly(oligo(ethylene glycol), methylether methacrylate, a carboxylic dextran, a hydrocarbon chain, a substituted hydrocarbon chain, a silane group, 3-mercaptopropyl triethoxysilane (MPTES), or a combination thereof.

In some aspects, the universal enhancer solution includes nanoparticles, each nanoparticle being conjugated with one or more of the following: (a) an affinity tag that binds to an affinity tag on the donor probe or the donor antibody; (b) an anti-bioluminescent antibody that binds to a bioluminescent tag on the donor probe or the donor antibody; (c) an anti-fluorescent antibody that binds to a fluorescent tag on the donor probe or the donor antibody; or (d) an anti-chemiluminescent antibody that binds to a chemiluminescent tag on the donor probe or the donor antibody.

In some aspects, the detection system further includes a spacer between the nanoparticle and the anti-fluorescent/bioluminescent/chemiluminescent antibody or affinity tag.

In some aspects, the spacer includes polyethylene glycol (PEG), a polyamino acid, a polyacrylamide, polyvinylpyrrolidone, a zwitterionic polymer, a polysaccharide, poly(N-(2-hydroxypropyl)methacrylamide), poly(oligo(ethylene glycol), methylether methacrylate, a carboxylic dextran, a hydrocarbon chain, a substituted hydrocarbon chain, a silane group, 3-mercaptopropyl triethoxysilane (MPTES), or a combination thereof. In some aspects, the spacer has a thickness of 0.5 nm-20 nm.

In some aspects, the nanoparticle includes gold, silver, iron oxide, a quantum dot, a carbon-based nanoparticle, a chemical nanoparticle, a liposome, a polymeric nanoparticle, a dendrimer, a magnetic nanoparticle, a silica nanoparticle, a metal oxide nanoparticle, a semiconductor nanoparticle, an MXene nanoparticle, or a combination thereof. In some aspects, the nanoparticle is spherical, rod-shaped, triangular, prismatic, cube-shaped, star-shaped, wire-shaped, a sheet, tube-shaped, hollow or cage-like, flower-shaped, disk-shaped, a nanopyramid, a nanobipyramid, a nanoplate, a self-assembled nanostructure, a bowtie antenna, a nano island, a nanoshell, or a combination thereof. In some aspects, the nanoparticle is about 4 nm to about 2000 nm.

In some aspects, the thickness from the nanoparticle surface to the donor bioluminescent and fluorescence tag is about 5 nm to about 20 nm.

In some aspects, the donor probe includes a bioluminescent tag attached to the 5′ end to bind with the anti-bioluminescent antibody on the nanoparticle of the universal signal enhancer.

In some aspects, the bioluminescent tag is Luciferin, Luciferyl adenylate firefly luciferase, Renilla luciferase, aequorin, Gaussia luciferase, or bacterial luciferase, coelenterazine aequorin, dinoflagellate luciferin Photoprotein, nanoluc luciferase, cypridina luciferase, nanobit-smallbit, nanobit-largebit or a functional equivalent thereof.

In some aspects, the donor probe further includes an affinity tag attached to the 3′ end to bind with the affinity tag on the nanoparticle of the universal signal enhancer.

In some aspects, the donor antibody includes a bioluminescent tag to bind with the anti-bioluminescent antibody on the nanoparticle of the universal signal enhancer.

In some aspects, the bioluminescent tag is Luciferin, Luciferyl adenylate firefly luciferase, Renilla luciferase, aequorin, Gaussia luciferase, or bacterial luciferase, coelenterazine aequorin, dinoflagellate luciferin Photoprotein, nanoluc luciferase, cypridina luciferase, nanobit-smallbit, nanobit-largebit or a functional equivalent thereof.

In some aspects, the donor antibody further includes an affinity tag to bind with the affinity tag on the nanoparticle of the universal signal enhancer.

In another embodiment, the present disclosure is directed to a method for detection of a target analyte, including the detection system of the disclosure, wherein the method includes: (a) hybridizing the capture probe to the target analyte to form a capture probe-target analyte complex, wherein the capture probe includes a photocleavable spacer with a biotin label affinity tag; (b) attaching the biotin label to a magnetic bead; (c) separating the capture probe-target analyte complex with a magnet; (d) hybridizing a donor probe to the target analyte to form a capture probe-target analyte-donor probe complex, wherein the donor probe includes an affinity tag and a bioluminescent tag; (e) separating the capture probe-target analyte-donor probe complex with a magnet; (f) exposing the capture probe-target analyte-donor probe complex to ultraviolet light to detach the magnetic bead from the complex; (g) contacting the capture probe-target analyte-donor probe complex with acceptor probes on the nanoparticle array to create a BRET or FRET complex; (h) adding the universal signal enhancer solution of claim 1; and (i) introducing a substrate reagent to generate light emission.

In some aspects, the detection system further includes hybridizing an extended probe to the target analyte and capture probe.

In some aspects, the cleavable linker or spacer includes disulfide linkers, photocleavable linkers, acid-labile linkers, enzyme-cleavable linkers, or hydrazone linkers.

In some aspects, the bioluminescent tag on the donor probe and the fluorescent tag on the acceptor probe are positioned at a distance less than 10 nm apart.

In some aspects, the first fluorescent tag and the second fluorescent tag are positioned at a distance less than 10 nm apart.

In some aspects, the first bioluminescent subunit tag and the second bioluminescent subunit tag are positioned at a distance less than 10 nm apart.

In some aspects, the donor probe includes a no tag, and acceptor probe includes no tag, and the detection system generates a localized surface plasmon resonance (LSPR) signal.

In some aspects, the donor probe includes an affinity tag for interaction with a universal signal enhancer, and the acceptor probe includes no tag, and the detection system generates a localized surface plasmon resonance (LSPR) signal.

In some aspects, the universal signal enhancer solution increases the sensitivity of chemiluminescence detection by at least 1 to 1,000 or more times.

In some aspects, the universal signal enhancer solution containing one nanoparticle increases the sensitivity of standard chemiluminescence detection by at least about 10, 20, 30, 40 50 or more times.

### BRIEF DESCRIPTION OF THE DRAWINGS

FIG. 1 is a schematic diagram illustrating the application of the Multi-Omic integration platform (MIP) for detecting analytes from biospecimen samples using Method 1. It depicts the MIP assay for polynucleotide, small RNA, and protein analytes, showing the steps from initial hybridization/conjugation to final signal detection. FIG. 1.1 is a schematic diagram illustrating the application of MIP for polynucleotide detection using Method 1. FIG. 1.2 is a schematic diagram illustrating the application of MIP for small RNA detection using Method 1. FIG. 1.3 is a schematic diagram illustrating the application of MIP for protein detection using Method 1.

FIG. 2A-2E are schematic diagrams illustrating the MIP assay based on Method 1 for detecting polynucleotides, small RNA, and proteins. They show the platform setup, detection configurations for various analytes, and details of the universal signal enhancer solution.

FIG. 3 is a schematic diagram illustrating the application of the MIP for detecting analytes from biospecimen samples using Method 2. It shows the process for polynucleotide, small RNA, and protein analytes, from initial binding to final signal detection. FIG. 3.1 is a schematic diagram illustrating the application of MIP for polynucleotide detection using Method 2. FIG. 3.2 is a schematic diagram illustrating the application of MIP for small RNA detection using Method 2. FIG. 3.3 is a schematic diagram illustrating the application of MIP for protein detection using Method 2.

FIGS. 4A-4E are schematic diagrams of the MIP assay based on Method 2 for detecting polynucleotides, small RNA, and proteins. They illustrate the platform setup, detection configurations for various analytes, and components of the universal signal enhancer solution.

FIG. 5 illustrates a flowchart of the MIP design and optimized parameter conditions.

FIG. 6A illustrates a graph showing capturing efficiencies for various bead to ssDNA ratios. It displays ratios from 1:0.01 to 1:0.65 mg/mL beads per 1 μg/mL ssDNA, with the optimal concentration at a ratio of 0.25 mg/mL beads.

FIG. 6B illustrates a graph showing capturing efficiencies for various bead to antibody ratios. It shows ratios from 1:0.01 to 1:0.10 mg/mL beads per 10 μg/mL antibodies, with the highest efficiency at 0.05 mg/mL beads.

FIG. 6C illustrates a graph showing AuSPs and AuNRs absorption scattering profiles. It displays normalized extinction spectra of aqueous solutions of AuSPs (520 nm) and AuNRs (555 nm), highlighting overlap with NanoLuc emission and Oregon Green absorbance/emission peaks.

FIG. 6D illustrates a graph showing maximum single monolayer coverage of AuSP on the substrate. It presents the evaluation of AuSP coverage using solutions varying in optical density from 0.5 OD to 3.0 OD.

FIG. 6E illustrates a scanning electron microscopic image of AuSPs on the substrate, showing a maximum coverage of 60% and confirming single monolayer formation.

FIG. 6F illustrates a graph showing the determination of optimal concentrations for ssDNA-SH and antibodies for enhanced fluorescence. It displays results for concentrations ranging from 1 to 30 μg/mL for both OG-ssDNA-SH and antibodies OG-PEG-1K-SH.

FIG. 6G illustrates a graph showing distance-dependent fluorescence enhancement by plasmonic nanostructures. It demonstrates optimal distances from gold nanoparticles for nucleic acids and antibodies.

FIG. 6H illustrates a bar graph showing fluorescence enhancement comparing samples without nanoparticles, at approximately 8 nm, and greater than 20 nm distances from the AuSP nanoparticle array surface.

FIG. 6I illustrates a graph showing the development of a standard curve for ANXA10 mRNA under three conditions: no nanoparticles, one nanoparticle, and two nanoparticles at concentrations from 100 nM to 100 pM.

FIG. 6J illustrates a graph showing the development of a standard curve for NMP22 protein under three conditions: no nanoparticles, one nanoparticle, and two nanoparticles at concentrations from 100 nM to 100 pM.

FIG. 6K illustrates a graph showing BRET enhancement with respect to the distance from the AuNRs for ANXA10 and NMP22, based on the PEG chain length for AuNRs linked to S-PEGn-NH-anti Nanoluc-Ab.

FIG. 6L illustrates a graph showing the optimum AuNRs concentration for universal signal enhancer solution. It displays results for solutions of AuNRs-S-PEG-NH-anti NanoLuc antibodies with varying optical densities to optimize signal enhancement of the MIP assay.

FIG. 6M illustrates an image of FDTD simulation of electromagnetic field enhancement upon AuSP and AuNR coupling.

FIG. 7A illustrates a graph showing the LSPR peaks of AuSPs (520 nm) and AuNRs (650 nm) alongside the emission spectrum of Nanoluc (460 nm—Blue region) and the absorption (590 nm—Orange region) and emission (645 nm—dense and wavy pattern) spectra of Ligand 618.

FIG. 7B illustrates calibration plot for NMP22 (ranging from 100 nM to 100 aM) on the MIP assay, using the Nanoluc-Ligand 618 BRET pair (red triangles), the Nanoluc-OG BRET pair (blue circles), and the CY3-CY5 FRET pair (magenta squares).

FIG. 8A illustrates a schematic diagram of different readout signals using Localized Surface Plasmon Resonance (LSPR) with two nanoparticles for Method 1. Readout signals include BRET, FRET, bioluminescent, fluorescence, chemiluminescence, SERS, and LSPR. Method 1 uses a nanoparticle with an antibody that directly binds to the bioluminescent, fluorescent, or chemiluminescent tag.

FIG. 8B illustrates a schematic diagram of different readout signals using Localized Surface Plasmon Resonance (LSPR) with two nanoparticles for Method 2. Readout signals include BRET, FRET, bioluminescent, fluorescence, chemiluminescence, SERS, and LSPR. Method 2 uses a nanoparticle with an affinity tag that directly binds to the affinity tag on the donor probe or capture antibody.

FIG. 9 illustrates a graph showing the LSPR peaks of AuNPs and AuNRs alongside excitation and emission spectra of CY3 and CY5 acceptor tags.

FIGS. 10A-10B illustrate graphs showing calibration curves developed for MIP assay using Forster Resonance Energy Transfer (FRET) as a readout signal under three conditions for ABL1 (mRNA) and NMP22 (protein): Method 2 without nanoparticles for enhancement (blue triangle plot), with one nanoparticle for enhancement (red circle plot), and with two nanoparticles for enhancement (black square plot). FIG. 10A shows the FRET ratio using a FAM-tagged donor probe and a Cy5-tagged acceptor probe, corresponding to different concentrations of ABL1 (mRNA) analyte in accordance with the present disclosure. The limit of detection identified in the plot demonstrates an improvement of approximately 2 logs and 4 logs, respectively, compared to the FRET complex with one nanoparticle and no nanoparticles. FIG. 10B shows FRET ratio using a FAM-tagged donor antibody and a Cy5-tagged acceptor antibody, corresponding to different concentrations of NMP22 protein analyte in accordance with the present disclosure. The limit of detection identified in the plot demonstrates an improvement of approximately 2 logs and 4 logs, respectively, compared to the FRET complex with one nanoparticle and no nanoparticles.

FIG. 11 illustrates a graph showing calibration curves for the MIP assay using Bioluminescent as a readout signal under three conditions for ABL1 (mRNA).

FIG. 12 illustrates a graph showing calibration curves for the MIP assay using fluorescence as a readout signal under three conditions for ABL1 (mRNA).

FIG. 13 illustrates a graph showing localized surface plasmon resonance as a readout signal for the MIP assay without any label using Method 2 for ABL1 (mRNA).

FIGS. 14A-14D illustrate graphs showing calibration plots for mRNA, lncRNA, miRNAs, and Proteins in spiked human urine samples using Method 1.

FIG. 15 illustrates a bar graph showing MIP assay specificity for each multi-omics category: protein, mRNA, miRNA, and lncRNA.

FIG. 16A illustrates a graph displaying the reproducibility tested for each omic category: protein (NMP22), miRNA (16-1), mRNA (ANXA10-1) and lncRNA (UCA) at varying concentrations. BRET ratios for these biomarkers were recorded after hybridization/conjugation on three independently prepared assay batches with each tested at four different concentrations. Each concentration was subjected to six technical replicates.

FIG. 16B illustrate a table showing the average coefficients of variation (CVs) for the concentrations of 100 nM, 100 pM, 100 fM, and 100 aM, which were found to be less than 2.88%, 2.92%, 1.95%, and 2.13% respectively.

FIG. 17 illustrates a graph showing the repeatability and reproducibility of the MIP assay across different laboratories and operators.

FIG. 18A and FIG. 18B illustrate graphs comparing the sensitivity and linearity of multi omic measurements using MIP assay technology against conventional RT-PCR for nucleic acids and ELISA for proteins.

FIGS. 19A-D illustrate box plots showing the expression levels of multi omicmarkers in urine from patients with bladder cancer tumors present and healthy individuals

FIGS. 20A-D illustrate receiver operating characteristic (ROC) curves for each biomarker predicting the presence of tumors.

FIGS. 21A-E illustrate scanning electron microscope (SEM) images of various nanoparticles and binding events in the MIP assay.

FIG. 22A illustrates a schematic representation of a standard sandwich ELISA. The figure depicts the sequence of steps involved in the assay: coating of capture antibody, addition of target specimen, binding of detection antibody with biotin tag, attachment of HRP-streptavidin conjugate, and introduction of chemiluminescent substrate. The figure visually represents the layered structure of antibodies, antigens, and enzymes that form in a standard sandwich ELISA, culminating in the production of luminescence for detection. Chemiluminescent substrate is added to complete the assay.

FIG. 22B illustrates a schematic representation of a standard sandwich ELISA with the addition of a universal signal enhancer (USE). This figure shows all the steps from FIG. 22A, plus an additional step where a USE solution containing AuNRs (gold nanorods) conjugated with an anti-HRP antibody is added. The figure visually represents how the USE solution integrates into the ELISA structure, binding to the HRP enzyme, and illustrates how this additional step enhances the sensitivity of the standard sandwich ELISA by amplifying the signal produced when the chemiluminescent substrate is added.

FIG. 23 illustrates a bar graph comparing IL-8 protein marker detection using standard sandwich ELISA and sandwich ELISA with universal signal enhancer (USE).

FIG. 24 illustrates a schematic representation of MIP technology used in cell and extracellular vesicle capture applications.

FIG. 25 is a table that provides an illustrative list of sequences for capture probes, donor probes, acceptor probes, and antibodies utilized in the MIP assay for the simultaneous detection of mRNA and protein targets.

### DETAILED DESCRIPTION OF THE INVENTION

This invention is not limited to the particular compositions, methods, and experimental conditions described, as such compositions, methods, and conditions may vary. Additionally, the terminology used herein is for the purposes of describing particular embodiments only, and is not intended to be limiting, since the scope of the present invention will be limited only in the appended claims.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The methods and materials are now described herein.

As used in this specification and the appended claims, the singular forms “a”, “an”, and “the” include plural references unless the context clearly dictates otherwise. Thus, for example, references to “the method” includes one or more methods and/or steps of the type described herein which will become apparent to those persons skilled in the art upon reading this disclosure.

### Multi-Omic Integration Platform (MIP)

Cancer is a heterogeneous and rapidly evolving disease. Achieving a precise diagnosis with a single biomarker, or from multiplexing the same type of marker, is challenging. The present disclosure describes a multi-omics approach to enhance the overall accuracy of cancer detection, particularly in early-stage cancers when they are most curable. This approach also allows description of a more comprehensive profile of disease signaling pathways and subtypes.

Conventional molecular diagnostic assays rely on complex and expensive instrumentation to identify specific target analytes, such as nucleic acids for PCR, or proteins for techniques like flow cytometry or mass spectrometry. This complexity poses a significant obstacle in integrating multi-omics approaches into both research and clinical settings.

Addressing this unmet need, the described Multi-Omic Integration Platform (MIP) uses advanced nanotechnology designed with a strong coupling of Localized Surface Plasmon Resonance (LSPR) of nanoparticles to enhance Bioluminescent Resonance Energy Transfer (BRET). This combination of LSPR and BRET allows for enhanced simultaneous detection and quantification of multi-omics (genomics, transcriptomics, proteomics, metabolomics, lipidomics) expression in a single platform.

### Multi-Omic Integration Platform (MIP): Method 1

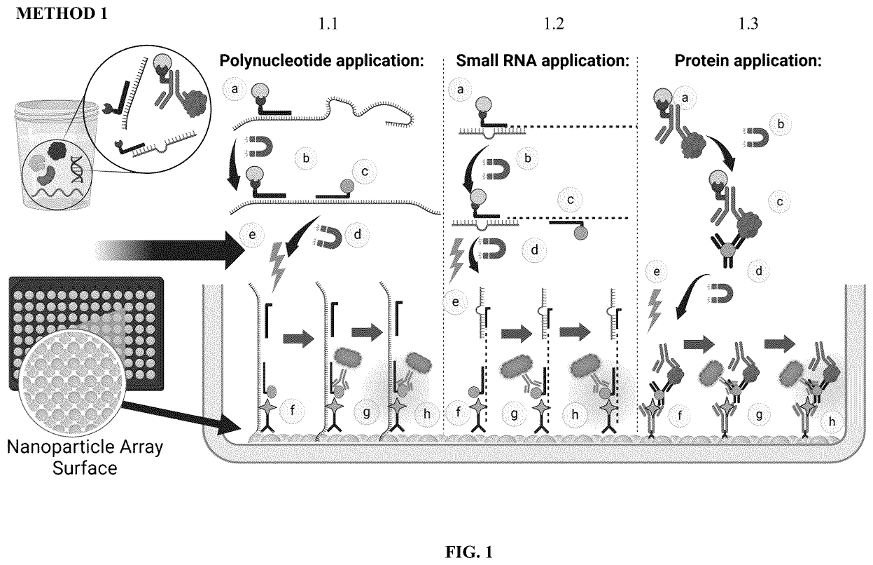

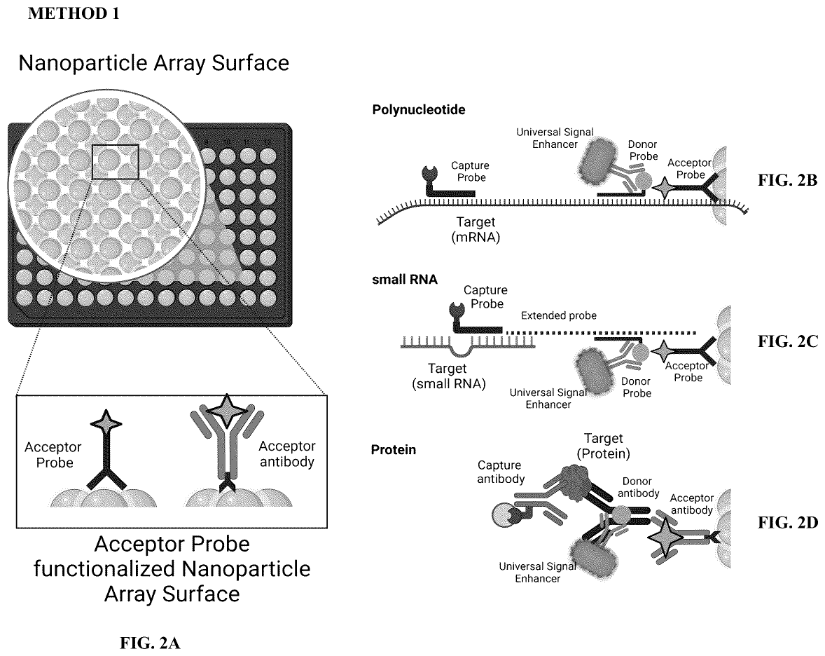

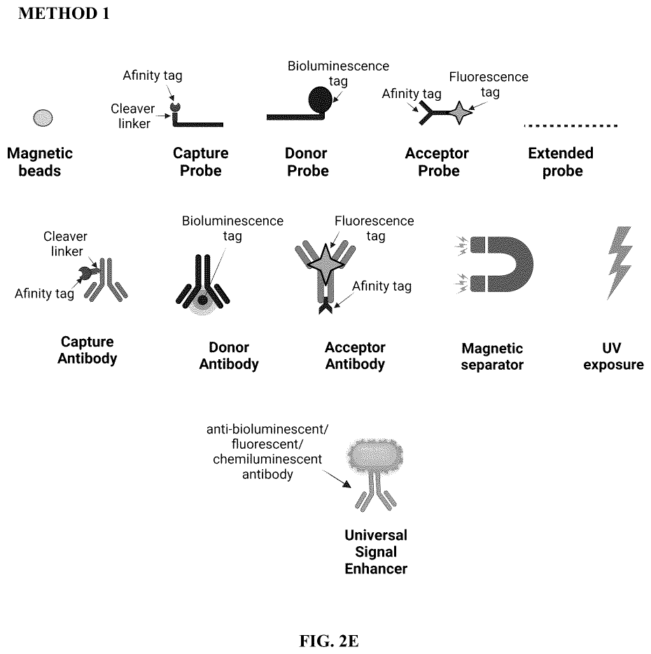

The disclosed MIP assay is designed for automation, enhancing both efficiency and accuracy. Utilizing capture antibodies tagged with a photocleavable biotin label and an affinity tag with an epitope binding site for the target protein, the assay captures analytes in biological samples through hybridization or conjugation reactions (FIGS. 2A-2E). This is followed by a magnetic bead-based separation of the analyte (FIG. 1). Subsequently, the polynucleotide analyte, mRNA or miRNA analyte (small RNA analyte), or protein analyte is introduced to a donor probe, or antibodies, and a second magnetic separation isolates the analyte-donor complex. UV exposure then facilitates the removal of magnetic beads. The analyte-donor solution is transferred to the nanoparticle array surface (Plasmo Matrix Array), where it forms a BRET complex with the acceptor antibody with fluorescent tag that contains binding sites for the donor antibody and is immobilized on the nanoparticle array surface. The next step involves the addition of a universal signal enhancer solution, that consists of a metallic nanoparticle with an anti-bioluminescent antibody to bind with the donor probe or the antibody bioluminescent tag (FIG. 2E). Finally, a single-addition reagent emits a glow-type signal for detection and quantification.

In some aspects, the donor probe or antibody includes a bioluminescent tag and the acceptor probe or antibody is untagged and the detection system creates a bioluminescent signal.

FIG. 2E is a schematic diagram of the components of a Multi-Omic Integration Platform (MIP) which illustrates how they are combined in order to detect a polynucleotide, a small RNA, and a protein. FIG. 1 is a schematic diagram which illustrates the application of one embodiment of the MIP to detect an analyte from a biospecimen sample. To detect a polynucleotide analyte from a biospecimen sample, a polynucleotide analyte bound to a capture probe with a photocleavable biotin affinity tag that has a magnetic bead at its 5′ (far right) end. The polynucleotide analyte is separated from the sample using magnetism and a magnetic bead. The donor probe is bound to the polynucleotide analyte. A second magnetic separation isolates the polynucleotide analyte-donor complex. UV exposure facilitates the removal of magnetic particles. The analyte-donor solution is transferred to the nanoparticle array surface, where it forms a BRET complex with the acceptor probe immobilized on the nanoparticle array surface. The universal signal enhancer (a metallic nanoparticle present in the universal signal enhancer solution) is added. The metallic nanoparticle, which has anti-bioluminescent antibody to bind with the bioluminescent tag of the donor probe. The single-addition reagent (luminescence substrate solution) emits a glow-type signal for detection and quantification.

To detect a small RNA analyte from the biospecimen sample, the small RNA is bound to capture probe with a photocleavable biotin affinity tag and an extended probe and purified using magnetism. Then a donor probe is bound to the extended probe, isolated with a second magnetic separation, and UV exposure facilitates the removal of the magnetic particles. The analyte-donor solution is transferred to the nanoparticle array surface and the analyte enhancer is added. The single-addition reagent emits a glow signal for detection and quantification.

To implement a magnetic bead-based separation process to isolate a protein or peptide of interest from a biospecimen sample, capture antibodies with a photocleavable biotin label and an affinity tag with an epitope binding site for the target analyte are introduced to the biospecimen sample and separated with magnetism. Donor antibodies that possess a bioluminescent tag and epitope binding sites for the above separated analyte via conjugation are introduced. A second magnetic separation step is performed to isolate the analyte-donor complex. Ultraviolet (UV) exposure facilitates the removal of magnetic particles from the analyte-donor complex. The analyte-donor complex solution is then transferred on to the nanoparticle array surface, that contains immobilized acceptor antibody with fluorescence tag and binding sites for the donor antibody. This conjugation leads to the formation of a Bioluminescent Resonance Energy Transfer (BRET) complex. An analyte enhancer solution is added. A single-addition reagent is added that emits a glow-type signal, enabling the detection and quantification of the target analyte.

In some aspects, the complex used to detect a polynucleotide includes the target polynucleotide (mRNA or DNA), a capture probe, a donor probe, an acceptor probe, and an analyte enhancer (FIG. 2B). In another aspect, the complex used to detect a small RNA target includes the target small RNA, a capture probe, and extended probe, a donor probe, an acceptor probe, and an analyte enhancer (FIG. 2C). In another aspect, the complex used to detect a protein target includes the target protein, a capture antibody, a donor antibody, and an acceptor antibody (FIG. 2D). These complexes are immobilized on a nanoparticle array surface (FIG. 2A).

By harnessing the high sensitivity of LSPR and the dynamic detection capabilities of BRET, the present Multi-Omic Integration Platform (MIP) revolutionizes the field of molecular diagnostics by providing a unified solution for detecting and quantifying a wide range of multi-omics biomarkers in a streamlined and accessible manner.

### Multi-Omic Integration Platform (MIP): Method 2

### Polynucleotides

Three sequence-specific probe hybridization techniques are described herein for the detection of polynucleotides including mRNA, lncRNA, DNA, and others. The first probe is a capture probe containing a 25 to 50 base sequence complementary to a particular target with a photo-cleavable spacer attached affinity tag such as biotin, or an amine or carboxylic acid. The second donor probe contains a 15-25 base sequence complementary to the target with bioluminescent tag to the 3′ end and affinity tag. The third acceptor probe, which is immobilized on the nanoparticle array surface through an affinity tag, contains a 10-25 base sequence complementary to the target with a acceptor fluorescence tag attached. Next, the addition of a universal signal enhancer solution binds to the affinity tag on the donor probe, completing the LSPR enhanced BRET complex. Finally, a single-addition reagent emits a glow-type signal for detection and quantification.

In some aspects, the MIP assay detects small RNA selected from short RNA, microRNA (miRNA), tiny non-coding RNAs (tncRNA), small modulatory RNA, small interfering RNAs (siRNAs), Piwi-interacting RNAs (piRNAs), transfer RNA-derived small RNAs (tsRNAs or tRFs), small nuclear RNAs (snRNAs), small nucleolar RNAs (snoRNAs), small RNA fragments derived from ribosomal RNAs (rRFs), small modulatory RNAs (smRNAs), small guide RNAs (sgRNAs), and small temporal RNAs (stRNAs).

In some aspects, the MIP assay sensitivity of at least 90%, at least 91%, at least 92%, at least 93%, at least 94%, at least 95%, at least 96%, at least 97%, at least 98% or at least 99%. In some aspects, the MIP assay shows specificity of about 65% to 100%, about 75% to 100%, about 85% to 100%, or about 95% to 100%. In some aspects, the MIP assay shows specificity of at least 65%, at least 70%, at least 75%, at least 80%, at least 85%, at least 90%, at least 95%, or about 100%. In some aspects, the MIP assay shows accuracy of about 65% to 100%, about 75% to 100%, about 85% to 100%, or about 95% to 100%. In another aspect, the MIP assay shows accuracy of at least 65%, at least 70%, at least 75%, at least 80%, at least 85%, at least 90%, at least 95%, or about 100%. In some aspects, the MIP assay shows PPV of about 55% to 65%, about 65% to 100%, about 75% to 100%, about 85% to 100%, or about 95% to 100%. In another aspect, the MIP assay shows PPV of at least 55%, at least 65%, at least 70%, at least 75%, at least 80%, at least 85%, at least 90%, at least 95%, or about 100%. In one embodiment, the MIP assay shows NPV of at least 90%, at least 91%, at least 92%, at least 93%, at least 94%, at least 95%, at least 96%, at least 97%, at least 98% or at least 99%. In some aspects, the MIP assay shows NPV of about 65% to 100%, about 75% to 100%, about 85% to 100%, or about 95% to 100%.

In some aspects, the concentration of target analyte used is 1 aM to 1 μM. In some aspects, the analyte concentration is about 1 aM, about 5 aM, about 10 aM, about 20 aM, about 30 aM, about 40 aM, about 50 aM, about 60 aM, about 70 aM, about 80 aM, about 90 aM, about 100 aM, about 200 aM, about 300 aM, about 400 aM, about 500 aM, about 600 aM, about 700 aM, about 800 aM, about 900 aM, or about 1 fM. In some aspects, the analyte concentration is about 1 to 100 aM, about 100-200 aM, about 200-300 aM, about 300-400 aM, about 400-500 aM, about 500-600 aM, about 600-700 aM, about 700-800 aM, about 800-900 aM, about 900 aM-1 fM, 1-100 fM, 100-200 fM, 200-300 fM, 300-400 fM, 400-500 fM, 500-600 fM, 600-700 fM, 700-800 fM, 800-900 fM, or 900 fM-1 pM. In some aspects, the analyte concentration is about 1 pM, about 5 pM, about 10 pM, about 20 pM, about 30 pM, about 40 pM, about 50 pM, about 60 pM, about 70 pM, about 80 pM, about 90 pM, about 100 pM, about 200 pM, about 300 pM, about 400 pM, about 500 pM, about 600 pM, about 700 pM, about 800 pM, about 900 pM, or about 1 nM. In some aspects, the analyte concentration is about 1 to 100 pM, about 100-200 pM, about 200-300 pM, about 300-400 pM, about 400-500 pM, about 500-600 pM, about 600-700 pM, about 700-800 pM, about 800-900 pM, about 900 pM-1 nM. In some aspects, the analyte concentration is about 1 pM, about 5 pM, about 10 pM, about 20 pM, about 30 pM, about 40 pM, about 50 pM, about 60 pM, about 70 pM, about 80 pM, about 90 pM, about 100 pM, about 200 pM, about 300 pM, about 400 pM, about 500 pM, about 600 pM, about 700 pM, about 800 pM, about 900 pM, or about 1 nM. In some aspects, the analyte concentration is about 1-100 nM, 100-200 nM, 200-300 nM, 300-400 nM, 400-500 nM, 500-600 nM, 600-700 nM, 700-800 nM, 800-900 nM, or 900 nM-1 uM.

In some aspects, the present disclosure is directed to a target analyte detection system including: (a) a capture probe including an affinity tag; (b) a donor probe including a bioluminescent tag or a first fluorophore tag or a first bioluminescent subunit tag (c) an acceptor probe including a fluorescence tag compatible with to the bioluminescent tag or a second fluorophore tag compatible with to the first fluorophore tag or a second bioluminescent subunit tag compatible with to the first bioluminescent subunit tag (d) a nanoparticle array including a substrate, a nanoparticle, the acceptor probe, and optionally a spacer between the nanoparticle and the acceptor probe; and (e) a universal signal enhancer solution comprising a universal analyte enhancer, a nanoparticle and an affinity tag, wherein the donor probe and the acceptor probe are a BRET signal pair, a FRET signal pair, a bioluminescent pair, or a fluorescence pair. In some aspects, the donor probe includes a bioluminescent tag and the acceptor probe includes a fluorescence tag, and the detection system includes a bioluminescent resonance energy transfer (BRET) complex. In some aspects, the bioluminescent tag and the fluorophore tag on the acceptor probe are positioned at a distance less than 10 nm apart. In some aspects, the donor probe includes a first fluorophore tag and the acceptor probe includes a second fluorophore tag compatible with the first fluorophore tag, and the detection system includes a fluorescence resonance energy transfer (FRET) complex. In some aspects, the first fluorophore tag and the second fluorophore are positioned at a distance less than 10 nm apart. In some aspects, the donor probe includes a first bioluminescent subunit tag and the acceptor probe includes a second bioluminescent subunit tag and the detection system includes a bioluminescent signal. In some aspects, the first bioluminescent subunit tag on the donor probe and the second bioluminescent subunit tag on the acceptor probe are positioned at a distance less than 10 nm apart. In some aspects, the system further includes a universal signal enhancer solution including a nanoparticle and an affinity tag.

In some aspects, the donor probe includes a bioluminescent tag and the acceptor probe or the acceptor is untagged and the detection system creates a bioluminescent signal.

In some aspects, the detection system includes a universal signal enhancer solution including a nanoparticle and an affinity tag. In another aspect, the wherein the BRET signal pair, the FRET signal pair, the bioluminescent signal pair, or the fluorescence signal pair is located between 5-20 nm from nanoparticles of the plasmon matrix array. In a further aspect, the BRET signal pair, the FRET signal pair, the bioluminescent signal pair, or the fluorescence signal pair is located between 5-20 nm from nanoparticles of the universal analyte enhancer. In yet another aspect, signal enhancement is achieved using LSPR of the nanoparticles of nanoparticle array surface and the nanoparticles of the universal analyte enhancer. In some aspects, signal enhancement achieved using LSPR of the nanoparticles of nanoparticle array surface only. In another aspect, signal enhancement is achieved using LSPR of only the nanoparticles of the universal analyte enhancer. In yet another aspect, analyte detection can be used without enhancement from nanoparticles of the nanoparticle array or nanoparticles of the universal analyte enhancer.

In some aspects, an LSPR-Enhanced BRET assay that conducts simultaneous multi-omic detection via liquid biopsy, non-invasively, without extraction or amplification. The process begins with a biospecimen sample in a sample container, followed by the introduction of a capture probe and a donor probe that selectively bind to the target mRNA (a polynucleotide analyte) to form a complex that is brought into proximity with the nanoparticle array surface functionalized with the acceptor probe. The addition of the Universal Analyte Enhancer enhances the signal, and the final detection limit is indicated by a series of dots on the right, showing a progression from current ng/ml to a new, more sensitive fg/ml detection limit. The entire process illustrates the integration of various components leading to the enhanced detection of target mRNA.

### Polynucleotide Application

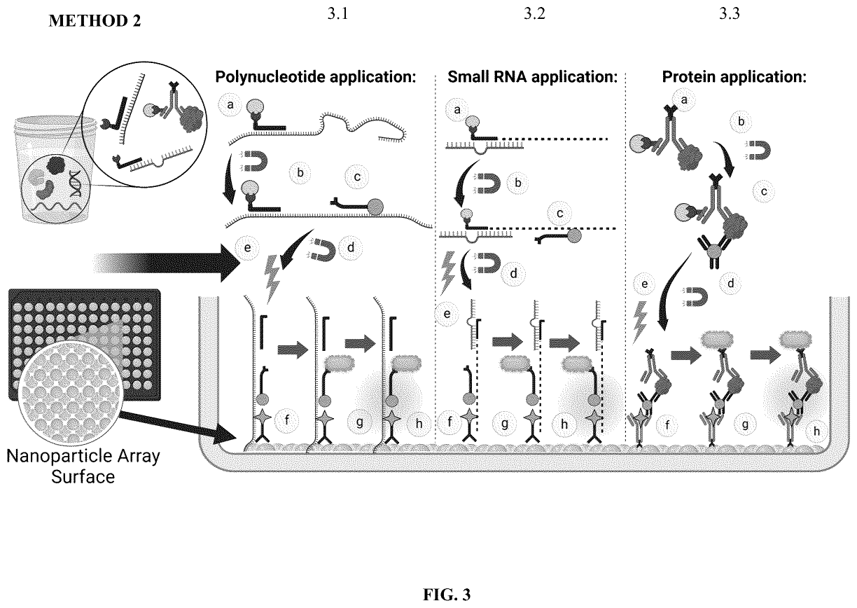

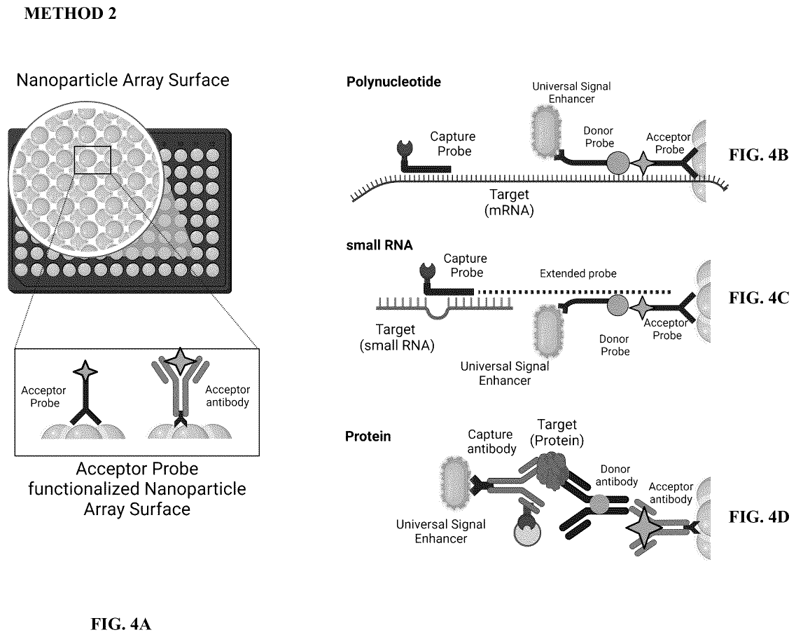

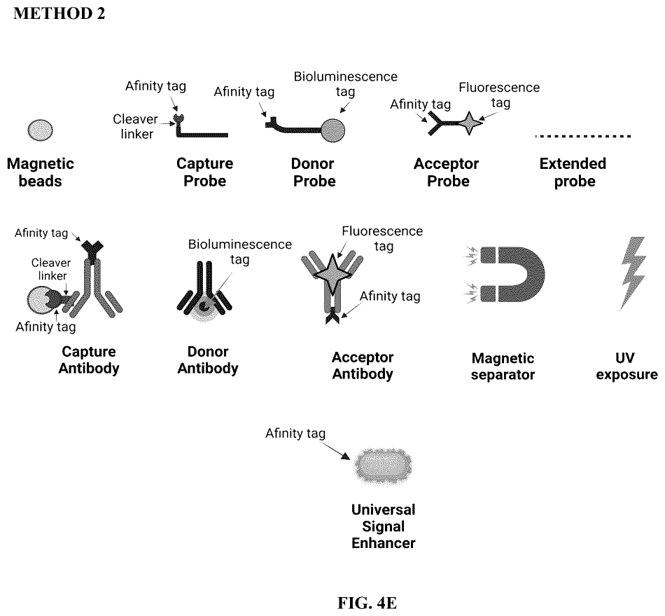

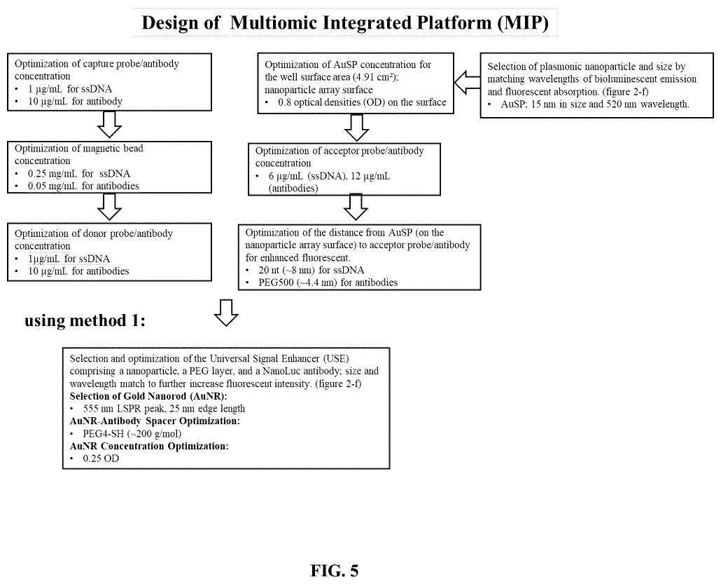

To detect a polynucleotide analyte (FIG. 3.1 and FIG. 4B) from a biospecimen sample, a polynucleotide analyte bound to a capture probe that has a magnetic bead at its 5′ (far right) end. The polynucleotide analyte is separated from the sample using magnetism and a magnetic bead. The donor probe is bound to the polynucleotide analyte. A second magnetic separation isolates the polynucleotide analyte-donor complex. UV exposure facilitates the removal of magnetic particles. The analyte-donor solution is transferred to the nanoparticle array surface (Plasmo Matrix Array Surface), where it forms a BRET complex with the acceptor probe immobilized on the nanoparticle array surface. The Universal Analyte Enhancer (a metallic nanoparticle present in the universal signal enhancer solution) is added. The metallic nanoparticle, which has binding sites for the donor probe, is shown bound to the donor probe that is part of the BRET complex. The single-addition reagent (luminescence substrate solution) emits a glow-type signal for detection and quantification.

### Small RNA Application

The disclosure provides utilized hybridization and ligation techniques to enhance the chain length of small RNA (miRNA, siRNA, piRNA, snRNA, snoRNA, tRFs, scaRNAs) for detection using four probe hybridization techniques. The capturing process involves two probe sequences, both of which are partially complementary to the small RNA of interest. The capture probe 1 (FIG. 4E) includes 6-20 base sequences complementary to half of the target small RNA with a photo-cleavable spacer (cleaver linker or cleavable linker) attached affinity tag, such as biotin. The Capture Probe 2, also known as the extended probe (FIG. 4E), includes a sequence of 20-100 bases. Within this sequence, approximately 6-20 bases are complementary to the remaining portion of the small RNA sequence of interest. The remaining bases of the extended probe are designed to complement the donor probe and the acceptor probe. The donor probe contains a 15-25 base sequence complementary to the extended probe with bioluminescent tag to the 3′ end and affinity tag to the 5′ end (FIG. 4E). The acceptor probe which is immobilized in the nanoparticle array through an affinity tag surface contains a 10-25 base sequence complementary to the target miRNA extended probe with acceptor fluorescence tag attached. Next, the addition of a universal signal enhancer solution binds to the affinity tag on the donor probe, completing the LSPR enhanced BRET complex. Finally, addition of a single-addition reagent emits a glow-type signal for detection and quantification.

### Protein or Peptide Application

Three different antibody systems are described herein; namely, a capture antibody, a donor antibody and an acceptor antibody for the protein or peptide detections. Capture antibodies for the protein or peptide of interest contain an affinity tag, such as HaloTag® linker label, and an affinity tag such as biotin attached to a photo-cleavable spacer, or cleaver linker (FIG. 4E). The donor antibody contains a bioluminescent tag with a high affinity epitope to the target of interest (FIG. 4E). In some aspects, the acceptor antibody, which is immobilized on the nanoparticle array surface through an affinity tag (FIG. 4E), has affinity to the donor antibody with an acceptor fluorescence tag attached. Next, the addition of a universal signal enhancer solution binds to the affinity tag on the capture antibody, completing the LSPR enhanced BRET complex. Finally, a single-addition reagent emits a glow-type signal for detection and quantification.

### Multi-Omic Integration Platform (MIP) Characterization

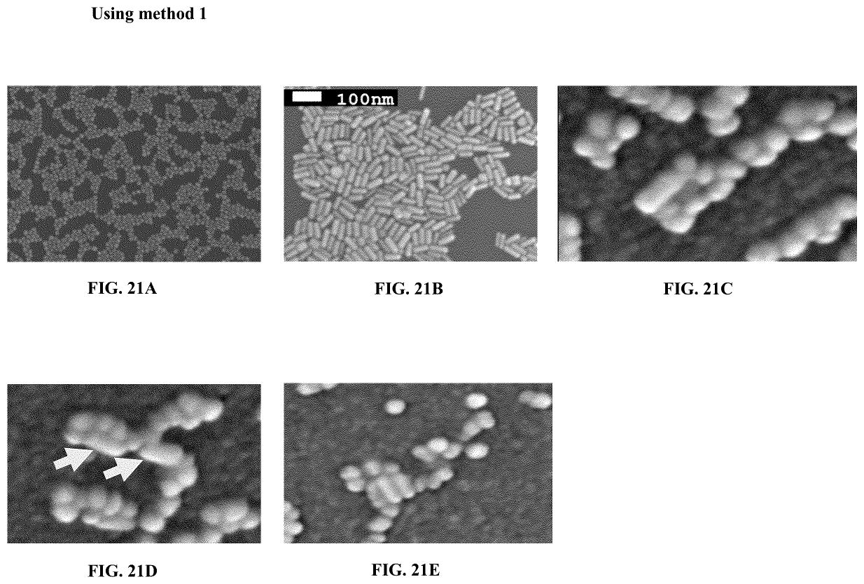

The nanoparticle array is composed of a glass substrate that can contains self-assemble monolayer of nanoparticles as its foundation (FIG. 21A). This array is compatible with a wide range of standard and specialized bioanalytical platforms. In some aspects, the array includes use of a 96 well plate with 5 mm surface area (FIG. 21B). In some aspects the array can be 100 um to 20 cm. In some aspects array can be 2D. In some aspects array can be 1 um to 5 mm.

In some aspects, the substrate includes a nanofiber array, glass, an elastomeric polymer, polydimethylsiloxane (PDMS), silicon wafer or other semiconductor material ECOFLEX®, SILBIONE®, polyethylene terephthalate (PET), polyurethane (PU), polyethylene naphthalate (PEN), a polyimide (PI), polybutadiene, polyisoprene, a silane, a polyamine, polymethylmethacrylate (PMMA), polydopamine, polyvinylpyrrolidone (PVP), polyvinyl alcohol (PVA), a polyolefin, a polyamide, a polyimide, a protein, silk, cellulose, a polyelectrolyte, a peptoid, or a combination thereof. In some aspects, the nanoparticle includes gold, silver, iron oxide, a quantum dot, a carbon-based nanoparticle, a chemical nanoparticle, a liposome, a polymeric nanoparticle, a dendrimer, a magnetic nanoparticle, a silica nanoparticle, a metal oxide nanoparticle, a semiconductor nanoparticle, an MXene nanoparticle, latex beads or a combination thereof. In some aspects, the nanoparticle is spherical, rod-shaped, triangular, prismatic, cube-shaped, star-shaped, wire-shaped, a sheet, tube-shaped, hollow or cage-like, flower-shaped, disk-shaped, a nanopyramid, a nanobipyramid, a nanoplate, a self-assembled nanostructure, a bowtie antenna, a nano island, a nanoshell, or a combination thereof. In some aspects, the nanoparticle is about 4 nm to about 2000 nm. In some aspects, the spacer between the nanoparticle and the acceptor probe has a thickness from about 0.5 nm to about 20 nm. In some aspects, the spacer has a thickness from about 0.5 nm to 10 nm, about 10 nm to 15 nm, about 15 to 20 nm, or about 20-25 nm. In some aspects, the spacer includes polyethylene glycol (PEG), a polyamino acid, a polyacrylamide, polyvinylpyrrolidone, a zwitterionic polymer, a polysaccharide, poly(N-(2-hydroxypropyl) methacrylamide), poly(oligo(ethylene glycol), methylether methacrylate, a carboxylic dextran, a hydrocarbon chain, a substituted hydrocarbon chain, a silane group, 3-mercaptopropyl triethoxysilane (MPTES), or a combination thereof.

### Polynucleotides

Compositions of the disclosure include polynucleotides. As used herein, polynucleotides refer to a substance that contains a polymer of nucleotides e.g., linked nucleosides. As a non-limiting example, the polynucleotide is a ribonucleic acid (RNA), a ribonuclear particle, a circular RNA, a deoxyribonucleic acid (DNA), cDNA, mRNA, iRNA, miRNA, tRNA, ncRNA, rRNA, short RNA, siRNA, microRNA (miRNA), tiny non-coding RNA (tncRNA) and small modulatory RNA (smRNA) small RNA (miRNA, siRNA, piRNA, snRNA, snoRNA, tRFs, scaRNAs).

In some aspects, the target analyte is a nucleic acid or a protein. In some aspects, the nucleic acid is DNA, lncRNA, mRNA, tRNA, rRNA, or small RNA. In some aspects, the techniques described herein relate to a system, wherein the nucleic acid is small RNA. In some aspects, the small RNA is an small interfering RNA (siRNA), a Piwi-interacting RNA (piRNA), a transfer RNA-derived small RNA (tsRNA or tRF), a small nuclear RNA (snRNA), a small nucleolar RNA (snoRNA), a small RNA fragment derived from ribosomal RNA (rRF), a small modulatory RNA (smRNA), a small guide RNA (sgRNA), a small temporal RNA (stRNA), a microRNA, a tiny non-coding RNAs (tncRNA), or a small modulatory RNA.

In some aspects, the system further includes an extended probe to lengthen the small RNA. In some aspects, the extended probe includes a sequence of about 25 to 100 nucleotides, wherein about 6 to 20 nucleotides are complimentary to a portion of the small RNA, and the remaining bases of the extended probe are complimentary to the donor probe and the acceptor probe.

In some aspects, the capture probe includes a nucleotide sequence from about 10 to 100 or more specifically, 25 to 50 nucleotides in length. In some aspects, the capture probe is complimentary to a target analyte. In some aspects, the capture probe includes an affinity tag with a cleavable spacer attached to the 5′ end of the capture probe, and optionally an extended probe.

In some aspects, the affinity tag includes His-tag, FLAG-tag, Glutathione S-transferase (GST) tag, Maltose Binding Protein (MBP) tag, Strep-tag, HA-tag, Myc-tag, Avi-tag, V5-tag, T7-tag, biotin or its derivatives, SNAP tag, CLIP tag, or HaloTag®. In some aspects, the affinity tag includes biotin or a functional derivative thereof.

In some aspects, the cleavable spacer includes a photocleavable spacer or a restriction enzyme binding site.

In some aspects, the donor probe includes a nucleotide sequence of about 10 to 100 nucleotides or about 15 to 25 nucleotides in length. In some aspects, the donor probe is complimentary to the target analyte. In some aspects, the donor probe is complimentary to the target extended probe. In some aspects, the donor probe includes a bioluminescent tag or a fluorophore tag or bioluminescent subunit attached to the 3′ end of the donor probe and an affinity tag attached to the 5′ to bind with the nanoparticle of the universal analyte enhancer. In some aspects, the donor probe includes a bioluminescent tag or a fluorophore tag or bioluminescent subunit attached to the 3′ end of the donor probe. In some aspects, the bioluminescent tag is Luciferin, Luciferyl adenylate firefly luciferase, Renilla luciferase, aequorin, Gaussia luciferase, or bacterial luciferase, coelenterazin aequorin, dinoflagellate luciferin Photoprotein, nanoluc luciferase, cypridina luciferase, nanobit-smallbit, nanobit-largebit or a functional equivalent thereof.

In some aspects, the acceptor probe is a nucleotide sequence from about 6 to 100 nucleotides or about 10 to 25 nucleotides in length. In some aspects, the acceptor probe is complimentary to the target analyte. In some aspects, the acceptor probe is complimentary to the extended probe. In some aspects, a fluorescence tag or bioluminescent subunit is attached to the 5′ end of the acceptor probe. In some aspects, the fluorescence tag is Oregon green and ligand 18, green fluorescent protein, a red fluorescent protein, a fluorescent dye, a quantum dot, an organic dye, Alexa Fluor dye, DyLight Fluor dye, BODIPY dye, Allophycocyanin, Phycoerythrin, a fluorescent nanoparticle, or lanthanide chelate. In some aspects, the fluorescence tag is Oregon green and ligand 18. In some aspects, the bioluminescent tag is firefly luciferase, Renilla luciferase, aequorin, Gaussia luciferase, or bacterial luciferase, Nanoluc luciferase, Cypridina luciferase, smallBIt, largeBit. In other aspects, the fluorescence tag or bioluminescent subunit is attached to the 3′ end of the acceptor probe. In some aspects, the acceptor probe is untagged.

In some aspects, the target analyte is a protein, and the capture probe is a capture antibody, the donor probe is a donor antibody, and the acceptor probe is an acceptor antibody. In some aspects, the capture antibody includes a photocleavable biotin label and an affinity tag to bind with the nanoparticle of the universal analyte enhancer. In some aspects, the affinity tag includes a His-tag, a FLAG-tag, a Glutathione S-transferase (GST) tag, a Maltose Binding Protein (MBP) tag, a Strep-tag, a HA-tag, a Myc-tag, an Avi-tag, a V5-tag, a T7-tag, biotin or its derivatives, a SNAP tag, a CLIP tag, or a HaloTag®.

In some aspects, the donor antibody includes a bioluminescent tag and epitope binding sites for the target analyte, and optionally an affinity tag to bind with the solution enhancer. In some aspects, the bioluminescent tag is Oregon green and ligand 18, green fluorescent protein, a red fluorescent protein, a fluorescent dye, a quantum dot, an organic dye, Alexa Fluor dye, DyLight Fluor dye, BODIPY dye, Allophycocyanin, Phycoerythrin, a fluorescent nanoparticle, or lanthanide chelate. In some aspects, the bioluminescent tag is firefly luciferase, Renilla luciferase, aequorin, Gaussia luciferase, or bacterial luciferase, Nanoluc luciferase, Cypridina luciferase, smallBIt, largeBit. In some aspects, the bioluminescent tag is luciferin or a functional equivalent thereof.

In some aspects, the acceptor antibody includes a fluorescence tag or a bioluminescent tag, an affinity tag to bind to the nanoparticle array, and binding sites for the donor antibody, wherein the donor antibody is complexed with the target analyte and the capture antibody. In some aspects, the acceptor antibody is bound to the donor antibody to create a bioluminescent resonance energy transfer (BRET) complex.

In some aspects, the acceptor antibody is bound to the target analyte and the donor antibody to create a fluorescence resonance energy transfer (FRET) complex. In another aspect, the acceptor antibody is bound to the donor antibody to create a fluorescence resonance energy transfer (FRET) complex.

In some aspects, the acceptor antibody is bound to the target analyte and the donor antibody to create a bioluminescent complex. In another aspect, the acceptor antibody is bound to the donor antibody to create a bioluminescent signal. In another aspect, the acceptor antibody is bound to the donor antibody to create a fluorescence signal.

Polynucleotides of the disclosure are recombinant polynucleotides. The term “recombinant polynucleotide,” as used herein, is defined as a polynucleotide that is not in its native state, e.g., the polynucleotide includes a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acids.

In some aspects, polynucleotides of the present disclosure are modified. As used herein, the terms “modified”, or as appropriate, “modification” refers to chemical modification with respect to A, G, U (T in DNA) or C nucleotides. Modifications are on the nucleoside base and/or sugar portion of the nucleosides which include the polynucleotide. In some embodiments, multiple modifications are included in the modified nucleic acid or in one or more individual nucleosides or nucleotides. For example, modifications to a nucleoside include one or more modifications to the nucleobase and the sugar.

In some aspects, the recombinant polynucleotides are present in vectors described herein. One or more recombinant polynucleotides of the disclosure are present in the same vector. Alternatively, each recombinant polynucleotide is present in a different vector. In some embodiments, the present disclosure includes at least a first recombinant polynucleotide and a second recombinant polynucleotide. The first recombinant polynucleotide encodes an RNA payload, a 5′UTR and a packaging signal. In one embodiment, the first recombinant polynucleotide includes a 3′UTR. Additionally, the first recombinant polynucleotide can be operably linked to a first promoter. In some embodiments the first polynucleotide can include a DNAzyme binding site or a ribozyme recognition sequence. The second recombinant polynucleotide can encode a packaging protein and a second 5′UTR. The second polynucleotide can be operably linked to a second promoter. In one embodiment, the recombinant polynucleotides include one or more enhancers or translational repressors.

The term “fragment” refers to any polypeptide having an amino acid residue sequence shorter than that of a polypeptide whose amino acid residue sequence is disclosed herein.

As used herein, the term “nucleic acid” or“oligonucleotide” refers to polynucleotides such as deoxyribonucleic acid (DNA) or ribonucleic acid (RNA). Nucleic acids include but are not limited to genomic DNA, cDNA, mRNA, piRNA, miRNA, tRNA, ncRNA, rRNA, short RNA, siRNA, microRNA (miRNA), tiny non-coding RNA (tncRNA) and small modulatory RNA (smRNA), and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid is isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e., transfection of, cells, in particular, in the form of RNA which is prepared by in vitro transcription from a DNA template. The RNA can moreover be modified before application by stabilizing sequences, capping, and polyadenylation.

Generally, nucleic acid is extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663.

In some aspects, the analytes of this invention are captured without the need for extraction or amplification.

In some aspects, the molecules of this invention are synthesized using recombinant DNA methods well described in the art.

The terms “peptide”, “polypeptide” and “protein” are used interchangeably herein and refer to any chain of at least two amino acids linked by a covalent chemical bound. As used herein, a peptide refers to the complete amino acid sequence coding for an entire protein or to a portion thereof. A “protein coding sequence,” or a sequence that “encodes” a particular polypeptide or peptide, is a nucleic acid sequence that is transcribed, in the case of DNA, and is translated, in the case of mRNA, into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

### Antibodies

As used herein, the term “antibody” includes natural or artificial mono- or polyvalent antibodies including, but not limited to, polyclonal, monoclonal, multispecific, human, humanized or chimeric antibodies, single chain antibodies, Fab fragments. F(ab′) fragments, fragments produced by a Fab expression library, anti-idiotypic (anti-Id) antibodies, and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin can be of any type (e.g., IgG, IgE, IgM, IgD, IgA, and IgY), class (e.g., IgG1, IgG2, IgG3, IgG4, IgA1, and IgA2) or subclass of immunoglobulin molecule.

An antibody as disclosed herein includes an antibody fragment, such as, but not limited to, Fab, Fab′ and F(ab′)2, Fd, single-chain Fvs (scFv), single-chain antibodies, disulfide-linked Fvs (sdfv) and fragments including either a VL or VH domain. In one embodiment, the targeting moiety is an antibody or scFv.

An antigen-binding antibody fragment, including single-chain antibody, may include the variable region(s) alone or in combination with the entirety or a portion of the following: hinge region, CH1, CH2, and CH3 domains. In one embodiment, an antigen-binding fragment also includes any combination of variable region(s) with a hinge region, CHI, CH2, and CH3 domains. Also included is a Fc fragment, antigen-Fc fusion proteins, and Fc-targeting moiety. In some aspects, the antibody is from any animal origin including birds and mammals. In some aspects, the antibody is, or is derived from, a human, murine (e.g., mouse and rat), donkey, sheep, rabbit, goat, guinea pig, camel, horse, or chicken. Further, In some aspects, such antibody is a humanized version of an antibody. In another aspect, the antibody is monospecific, bispecific, trispecific, or of greater multispecificity.

The antibody herein specifically includes a “chimeric” antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA. 81:6851-6855). A chimeric antibody of interest herein includes “primatized” antibodies including variable domain antigen-binding sequences derived from a non-human primate (e.g., Old World Monkey, Ape etc) and human constant region sequences.

Various methods have been employed to produce antibodies. Hybridoma technology, which refers to a cloned cell line that produces a single type of antibody, uses the cells of various species, including mice (murine), hamsters, rats, and humans. Another method to prepare an antibody uses genetic engineering including recombinant DNA techniques. For example, antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen.

In some aspects, an antibody fragment includes a portion of an intact, antibody, e.g. including the antigen-binding or variable region thereof. Examples of antibody fragments include Fab, Fab′, F(ab′)2, and Fv fragments; Fc fragments or Fc-fusion products; diabodies; linear antibodies; single-chain antibody molecules; and multispecific antibodies formed from antibody fragment(s).

An intact antibody is one which includes an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof or any other modified Fc (e.g. glycosylation or other engineered Fc).

In some aspects, the capture antibody, donor antibody, or acceptor antibody, is an antibody or an antibody fragment.

In some aspects, a peptide of the present invention is synthesized by any of the techniques that are known to those skilled in “the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. An excellent summary of the many techniques available can be found in Steward et al., “Solid Phase Peptide Synthesis”* W. H. Freeman Co., San Francisco, 1969; Bodanszky, et al., “Peptide Synthesis”, John Wiley & Sons, Second Edition, 1976; J. Meienhofer, “Hormonal Proteins and Peptides”. Vol. 2. p. 46, Academic Press (New York), 1983; Merrifield, Adv. Enzymol., 32:221-96, 1969; Fields et al., Int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis. Appropriate protective groups usable in such synthesis are described in the above texts and in J. F. W. McOmie, “Protective Groups in Organic Chemistry”, Plenum Press, New York, 1973.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

In some aspects, the present disclosure is directed to a system for detecting a target analyte including a capture probe, a donor probe, an acceptor probe, and a nanoparticle array (Plasmo Matrix Array) surface. In some aspects, the system further includes a universal signal enhancer solution. In some aspects, the target analyte is a nucleic acid or a protein. In some aspects, the target analyte is DNA, lncRNA, mRNA, tRNA, rRNA, or small RNA. In some aspects, the nucleic acid is small RNA. In some aspects, the small RNA is an small RNA (miRNA, siRNA, piRNA, snRNA, snoRNA, tRFs, scaRNAs), siRNA, microRNA, tiny non-coding RNAs (tncRNA), or small modulatory RNA. In some aspects, the system further includes an extended probe to lengthen the small RNA. In some aspects, the extended probe includes a sequence of 20-100 nucleotides, 25-100 nucleotides, 35-100 nucleotides, 45-100 nucleotides, 50-100 nucleotides, 60-100 nucleotides, 70-100 nucleotides, or 80-100 nucleotides, wherein about 6-20 nucleotides are complimentary to a portion of the small RNA, and the remaining bases of the extended probe are complimentary to the donor probe and the acceptor probe.

In some aspects, the capture probe is a nucleotide sequence from about 10 to 100 or more specifically, about 25 to 50 nucleotides in length. In some aspects, the capture probe is a nucleotide sequence from about 5-15, 10-20, 20-30, 30-40, 40-50, 50-60, 60-70, 70-80, 80-90, or 90-100 nucleotides in length. In some aspects, the capture probe is a nucleotide sequence from about 10-50, 50-100, 20-50, 30-50, 40-50, 40-60, 40-70, 50-70, 50-80, or 50-90 nucleotides in length. In some aspects, the capture probe is complimentary to a target analyte. In some aspects, the capture probe includes an affinity tag attached to the 5′ end of the capture probe. In some aspects, the affinity tag includes His-tag, FLAG-tag, Glutathione S-transferase (GST) tag, Maltose Binding Protein (MBP) tag, Strep-tag, HA-tag, Myc-tag, Avi-tag, V5-tag, T7-tag, biotin or its derivatives, SNAP tag, CLIP tag, or HaloTag®. In some aspects, the affinity tag includes biotin or any of its functional equivalents.

In some aspects, the affinity tag includes an: amine, carboxylic acid, amino acid, thiol (SH), hydroxyl (OH), phosphate, azide group, alkaline group, ketone group, or a halide group, or a functional derivative thereof. In some aspects, the affinity tag includes biotin or a functional derivative thereof.

In some aspects, the donor probe is a nucleotide sequence of about 10-100 nucleotides or of about 15 to 25 nucleotides in length. In some aspects, the donor probe is a nucleotide sequence from about 5-15, 10-20, 20-30, 30-40, 20-25, 20-30, 30-40, 40-50, 50-60, or 60-70 nucleotides in length. In some aspects, the capture probe is a nucleotide sequence from about 10-50, 50-100, 20-50, 30-50, 40-50, 40-60, 40-70, 50-70, 50-80, 50-90, or 50-100 nucleotides in length. In some aspects, the donor probe is complimentary to the target analyte. In some aspects, the donor probe includes a bioluminescent tag attached to the 3′ end of the donor probe. In some aspects, the bioluminescent tag is firefly luciferase, Renilla luciferase, aequorin, Gaussia luciferase, or bacterial luciferase, Nanoluc luciferase, Cypridina luciferase, smallBIt, largeBit.

In some aspects, the acceptor probe is a nucleotide sequence from about 6 to 100 nucleotides or about 10 to 25 nucleotides in length. In some aspects, the acceptor probe is a nucleotide sequence from about 5-15, 10-20, 20-30, 30-40, 20-25, 20-30, 30-40, 40-50, 50-60, or 60-70 nucleotides in length. In some aspects, the acceptor probe is a nucleotide sequence from about 10-50, 50-100, 20-50, 30-50, 40-50, 40-60, 40-70, 50-70, 50-80, 50-90, or 50-100 nucleotides in length. In some aspects, the acceptor probe is complimentary to the target analyte. In some aspects, a fluorescence tag is attached to the 5′ end of the acceptor probe. In some aspects, the fluorescence tag is Oregon green and ligand 18, green fluorescent protein, a red fluorescent protein, a fluorescent dye, a quantum dot, an organic dye, Alexa Fluor dye, DyLight Fluor dye, BODIPY dye, Allophycocyanin, Phycoerythrin, a fluorescent nanoparticle, or lanthanide chelate. In some aspects, the fluorescence tag is Oregon green and ligand 18.

In some aspects, the target analyte is a protein, and the capture probe is a Capture Antibody, the donor probe is a Donor Antibody, and the acceptor probe is an Acceptor Antibody. In some aspects, the Capture Antibody includes a photocleavable biotin label and an affinity tag. In some aspects, the affinity tag includes a His-tag, a FLAG-tag, a Glutathione S-transferase (GST) tag, a Maltose Binding Protein (MBP) tag, a Strep-tag, a HA-tag, a Myc-tag, an Avi-tag, a V5-tag, a T7-tag, biotin or its derivatives, a SNAP tag, a CLIP tag, or a HaloTag®. In some aspects, the Donor Antibody includes a bioluminescent tag and epitope binding sites for the target analyte. In some aspects, bioluminescent tag is Oregon green and ligand 18, green fluorescent protein, a red fluorescent protein, a fluorescent dye, a quantum dot, an organic dye, Alexa Fluor dye, DyLight Fluor dye, BODIPY dye, Allophycocyanin, Phycoerythrin, a fluorescent nanoparticle, or lanthanide chelate. In some aspects, the bioluminescent tag is Oregon green and ligand 18 In some aspects, the Acceptor Antibody includes a fluorescence tag, an affinity tag to bind with the nanoparticle array, and binding sites for the donor antibody, wherein the donor antibody is complexed with the target analyte and the capture antibody. In some aspects, the Acceptor Antibody is bound to the target analyte and the Donor Antibody to create a Bioluminescent Resonance Energy Transfer (BRET) complex.

In some aspects, the Acceptor Antibody is bound to the Donor Antibody with the target analyte to create a fluorescence Resonance Energy Transfer (FRET) complex.

In some aspects, the Acceptor Antibody is bound to the Donor Antibody with the target analyte to create a Bioluminescent Complex.

In some aspects, the Acceptor Antibody is bound to the Donor Antibody with the target analyte to create a bioluminescent signal.

In some aspects, the Acceptor Antibody is bound to the Donor Antibody with the target analyte to create a fluorescence signal.

In some aspects, the nanoparticle array surface includes a substrate, a nanoparticle, the acceptor probe, and optionally a spacer between the nanoparticle and the acceptor probe. In some aspects, the substrate includes a custom nanofiber array, glass, an elastomeric polymer, polydimethylsiloxane (PDMS), ECOFLEX®, SILBIONE®, Silicone wafer or semiconductor materials (ITO), polyethylene terephthalate (PET), polyurethane (PU), polyethylene naphthalate (PEN), a polyimide (PI), polybutadiene, polyisoprene, a silane, a polyamine, polymethylmethacrylate (PMMA), polydopamine, polyvinylpyrrolidone (PVP), polyvinyl alcohol (PVA), a polyolefin, a polyamide, a polyimide, a protein, silk, cellulose, a polyelectrolyte, a peptoid, or a combination thereof. In some aspects, the nanoparticle includes gold, silver, iron oxide, a quantum dot, a carbon-based nanoparticle, a chemical nanoparticle, a liposome, a polymeric nanoparticle, a dendrimer, a magnetic nanoparticle, a silica nanoparticle, a metal oxide nanoparticle, a semiconductor nanoparticle, an MXene nanoparticle, or a combination thereof. In some aspects, the nanoparticle is spherical, rod-shaped, triangular, prismatic, cube-shaped, star-shaped, wire-shaped, a sheet, tube-shaped, hollow or cage-like, flower-shaped, disk-shaped, a nanopyramid, a nanobipyramid, a nanoplate, a self-assembled nanostructure, a bowtie antenna, a nano island, a nanoshell, or a combination thereof. In some aspects, the nanoparticle is about 4 nm to about 2000 nm. In some aspects, the nanoparticle is about 4 to 100 nm, about 100 to 200 nm, about 200-400 nm, about 400-600 nm, about 600-800 nm, about 800-1000 nm, about 1000-1500 nm, or about 1500 to 2000 nm. In some aspects, the spacer between the nanoparticle and the acceptor probe has a thickness from about 0.5 nm to about 20 nm. In some aspects, the spacer between the nanoparticle and the acceptor probe has a thickness from about 0.5 nm to 1 nm, about 1 nm to 2 nm, about 2 nm to 4 nm, about 4 nm to 6 nm, about 6 nm to 8 nm, about 8 to 10 nm, about 10 to 12 nm, about 12 to 14 nm, about 14 to 16 nm, about 16 to 18 nm, or about 18 to 20 nm or larger. In some aspects, the the spacer between the nanoparticle and the acceptor probe has a thickness from about 0.1 nm to 5 nm, about 5 nm to 10 nm, about 10 nm to 15 nm, or about 15 nm to 20 nm. In some aspects, the spacer includes polyethylene glycol (PEG), a polyamino acid, a polyacrylamide, polyvinylpyrrolidone, a zwitterionic polymer, a polysaccharide, poly(N-(2-hydroxypropyl)methacrylamide), poly(oligo(ethylene glycol), methylether methacrylate, a carboxylic dextran, a hydrocarbon chain, a substituted hydrocarbon chain, a silane group, 3-mercaptopropyl triethoxysilane (MPTES), or a combination thereof.

In some aspects, the universal signal enhancer solution is a solution including a nanoparticle with an affinity tag. In some aspects, the nanoparticle includes gold, silver, iron oxide, a quantum dot, a carbon-based nanoparticle, a chemical nanoparticle, a liposome, a polymeric nanoparticle, a dendrimer, a magnetic nanoparticle, a silica nanoparticle, a metal oxide nanoparticle, a semiconductor nanoparticle, an Mxene nanoparticle, latex beads or a combination thereof. In some aspects, the nanoparticle is spherical, a nanorod, rod-shaped, triangular, prismatic, cube-shaped, star-shaped, wire-shaped, a sheet, tube-shaped, hollow or cage-like, flower-shaped, disk-shaped, a nanopyramid, a nanobipyramid, a nanoplate, a self-assembled nanostructure, a bowtie antenna, a nano island, a nanoshell, or a combination thereof. In some aspects, the nanoparticle is about 1 nm to about 100 nm. In some aspects, the nanoparticle is a nanorod. In some aspects, the nanoparticle includes gold. In some aspects, the nanorod is bound to the affinity tag with or without a spacer. In some aspects, the nanorod has a length from about 25 nm to about 2000 nm, about 100 to 200 nm, about 200-400 nm, about 400-600 nm, about 600-800 nm, about 800-1000 nm, about 1000-1500 nm, or about 1500 to 2000 nm. In some aspects, the nanorod has a diameter from about 4 nm to about 100 nm. In some aspects, the spacer has a thickness from about 0.5 nm to about 20 nm. In some aspects, the spacer includes polyethylene glycol (PEG), a polyamino acid, a polyacrylamide, polyvinylpyrrolidone, a zwitterionic polymer, a polysaccharide, poly(N-(2-hydroxypropyl)methacrylamide), poly(oligo(ethylene glycol), methylether methacrylate, a carboxylic dextran, a hydrocarbon chain, a substituted hydrocarbon chain, a silane group, 3-mercaptopropyl triethoxysilane (MPTES), or a combination thereof. In some aspects, the universal signal enhancer solution increases the sensitivity of chemiluminescence detection by at least 1 time, at least 2 times, at least 5 times, at least 10 times, at least 20 times, at least 50 times, at least 100 times, at least 200 times, at least 500 times, and at least 1000 times. In some aspects, the universal signal enhancer solution containing one nanoparticle increases the sensitivity of standard chemiluminescence detection by at least 1 time, at least 2 times, at least 5 times, at least 10 times, at least 15 times, at least 20 times, at least 25 times, at least 30 times, at least 35 times, at least 40 times, at least 45 times, and at least 50 times.

In some aspects, the Multi-Omic Integration Platform (MIP) is used to detect markers indicative of disease such as cancer, infectious diseases, genetic mutations and disorders, and pathogens. In some aspects, the assay is used to detect COVID-19, influenza, tuberculosis, HIV, hepatitis C, cystic fibrosis, Huntington's disease, sickle cell anemia, human papillomavirus, Epstein-Barr virus, malaria, or anthrax. For example, a patient is tested for influenza, and when the assay is positive for influenza, the patient is treated with an antiviral medicine. In some aspects, the patient is treated with oseltamivir, baloavir, marboxil, zanamivir, or peramivir.

In some aspects, the MIP is used to test for cancer. In some aspects, the cancer is bladder cancer, head cancer, neck cancer, head and neck cancer, lung cancer, breast cancer, prostate cancer, colorectal cancer, esophageal cancer, gastric cancer, leukemia/lymphoma, uterine cancer, skin cancer, endocrine cancer, urological cancer, pancreatic cancer, gastrointestinal cancer, ovarian cancer, cervical cancer, or adenoma.

In some aspects, the MIP is configured for detecting a nucleic acid utilizing a set of three distinct probes, wherein the probes are selected from the group consisting of a capture probe, a donor probe, and an acceptor probe. In some aspects, the nucleic acid is DNA, lncRNA, mRNA, tRNA, or rRNA.

In some aspects, the MIP is configured for detecting small RNA chains utilizing hybridization and ligation techniques to extend the length of small RNA chains, wherein the extended length facilitates detection of the small RNA chains.

In some aspects, the MIP is configured for detection of a protein or peptide including a tripartite antibody system including a capture antibody, a donor antibody, and an acceptor antibody, wherein each antibody is designed for different roles in the detection of the protein or peptide.

In some aspects, the MIP of the present disclosure is directed to a system for detection of a nucleic acid, including two or more of a capture probe, an extended probe, a donor probe, and an acceptor probe. In some aspects, the techniques described herein relate to a system, wherein the capture probe, the donor probe, and the acceptor probe are antibodies.

In some aspects, the techniques described herein relate to a method for detection of a target analyte including the previously-described systems, wherein the method includes: a) hybridizing the capture probe to the target analyte to form a capture probe-target analyte complex, wherein the capture probe has a biotin label with a photocleavable or restriction endonuclease enzymes spacer; b) attaching the biotin label to a magnetic bead; c) separating the capture probe-target analyte complex with a magnet; d) hybridizing a donor probe to the target analyte to form a capture probe-target analyte-donor probe complex, wherein the donor probe includes an affinity tag and a bioluminescent tag; e) separating the capture probe-target analyte-donor probe complex with a magnet; f) exposing the capture probe-trarget analyte-donor probe complex to UV/enzyme to detach the magnetic bead from the complex; g) contacting the capture probe-target analyte-donor probe complex with the nanoparticle array containing immobilized acceptor probe with fluorescence tag to create a BRET complex; h) addition of the universal signal enhancer solution; to bind with affinity tag on the donor probe and i) introducing a substrate reagent to generate light emission.

In some aspects, the method further includes hybridizing the extended probe to the target analyte and capture probe, for use with detection of small RNA.

### Capture Probe

In some aspects, a capture probe includes a nucleotide sequence ranging from about 10 to 100 or 25 to 50 bases in length, designed to be complementary to a target analyte. In some aspects, the capture probe includes an affinity tag attached to the 5′ end of the probe via a photocleavable spacer, wherein the affinity tag includes biotin. In some aspects, the affinity tag includes a biotin-similar compound, and is not limited to biotin. In another aspect, the affinity tag does not include biotin. In some aspects, the affinity tag binds with streptavidin functionalized magnetic beads. In another aspect, the affinity tag is His-tag, FLAG-tag, Glutathione S-transferase (GST) tag, Maltose Binding Protein (MBP) tag, Strep-tag, HA-tag, Myc-tag, Avi-tag, V5-tag, T7-tag, biotin or its derivatives, SNAP tag, CLIP tag, HaloTag® or carboxylic acid or its derivatives, amine or its derivatives or others, which facilitates the binding of the magnetic beads with particular binding sites. In some aspects, the capture probe includes a photocleavable spacer. In some aspects, the photocleavable spacer is nitrobenzyl, nitrophenyl or their derivatives, coumarin-based linkers or their derivatives, benzoin esters or their derivatives, caged compounds or their derivatives, 2-nitrobenzyl alcohol derivatives, quinone-based spacers or their derivatives, azobenzene derivatives or their derivatives, or caged nucleotides or their derivatives. In some aspects, the photocleavable spacers are replaced by restriction endonucleases sequences such as EcoRI, HindIII, and BamHI, with 6-10 additional sequences, using restriction endonuclease enzymes to cleave the bond. In another aspect, the capture probe includes an affinity tag attached to the 3′ end of the probe via a photocleavable spacer, wherein the affinity tag includes biotin.

Various techniques are employed to bind biotin, amine, thiol, or other affinity groups to single-stranded DNA molecules or antibodies. In some aspects, biotinylating can be achieved through methods such as NHS-biotin labeling, DBCO-biotin click chemistry, or maleimide-biotin conjugation. In some aspect amine modification is commonly performed using NHS ester coupling, isocyanate coupling, or EDC/NHS coupling. In some aspects, thiol modification techniques include maleimide-thiol conjugation, haloacetamide-thiol conjugation, and thiol-disulfide exchange. In some aspects affinity group conjugation can be accomplished through carbodiimide coupling, sulfo-SMCC crosslinking, or DCC-mediated coupling. In some aspects, haloTag ligands to single-stranded DNA/antibodies can be achieved using methods such as amine modification followed by HaloTag ligand conjugation or direct conjugation of HaloTag ligands through NHS ester coupling or other suitable chemistries. In some aspects, we used commonly used technology/chemistries to probe/antibodies to functionalized with the target affinity tag.

### Donor Probe

In some aspects, a donor probe includes a nucleotide sequence or a bioluminescent tag. In some aspects, a donor probe includes a nucleotide sequence ranging from about 10-100 or 22 to 25 bases in length, complementary to the target analyte, forming the core of the second donor probe. In some aspects, a donor probe includes a bioluminescent tag attached to the 3′ end of the donor probe. In another aspect, the bioluminescent tag is NanoLuc (Engineered Luciferase). In another aspect, the donor probe includes Firefly Luciferase, Renilla Luciferase, Aequorin, Gaussia Luciferase, Bacterial Luciferase, Nanoluc luciferase, Cypridina luciferase, smallBIt, largeBit or another appropriate bioluminescent tag.

### Affinity Tag Linkage of the Donor Probe

In some aspects, a donor probe includes an affinity tag linker attached to the 5′ end of the donor probe. In some aspects, the affinity tag linker is a HaloTag® linker. The affinity tag is designed to bind with the Universal Analyte Enhancer. In another aspect, the affinity tag is a His-tag, a FLAG-tag, a Glutathione S-transferase (GST) tag, a Maltose Binding Protein (MBP) tag, a Strep-tag, an HA-tag, a Myc-tag, an Avi-tag, a V5-tag, a T7-tag, biotin or its derivatives, a thiol, amine or amine derivative, a carboxylic acid or carboxylic acid derivative, a SNAP tag, a CLIP tag, or other appropriate tag. The affinity tag facilitates the binding of the second donor probe to nanomaterials in a universal enhancement solution, enhancing the detection sensitivity and specificity; various chemical methods are employed to bind DNA strands to affinity tags such as the His-tag, FLAG-tag, GST-tag, MBP-tag, Strep-tag, HA-tag, Myc-tag, Avi-tag, V5-tag, T7-tag, biotin or its derivatives, SNAP tag, CLIP tag, or other appropriate tags. In some aspects, His-tagged DNA can be immobilized using Immobilized Metal Affinity Chromatography (IMAC), where metal ions such as Ni2+ or Co2+ coordinate with histidine residues in the His-tag. In some aspects FLAG-tagged DNA can be captured using anti-FLAG antibodies immobilized on a solid support. GST-tagged DNA can be bound to glutathione-coated surfaces through a GST affinity interaction. In some aspects, other tags like MBP, Strep-tag, HA-tag, Myc-tag, Avi-tag, V5-tag, and T7-tag can be immobilized using corresponding affinity chromatography methods or specific antibodies. In some aspects, biotinylating can be achieved through methods such as NHS-biotin labeling, DBCO-biotin click chemistry, or maleimide-biotin conjugation. In some aspect amine modification is commonly performed using NHS ester coupling, isocyanate coupling, or EDC/NHS coupling. In some aspects, thiol modification techniques include maleimide-thiol conjugation, haloacetamide-thiol conjugation, and thiol-disulfide exchange. In some aspects affinity group conjugation can be accomplished through carbodiimide coupling, sulfo-SMCC crosslinking, or DCC-mediated coupling. In some aspects, HaloTag ligands to single-stranded DNA/antibodies can be achieved using methods such as amine modification followed by HaloTag ligand conjugation or direct conjugation of HaloTag ligands through NHS ester coupling or other suitable chemistries. In some aspects, we used commonly used technology/chemistries to probe/antibodies to functionalize with the target affinity tag.

A method for introducing fluorescence tag and bioluminescent tag to single-stranded DNA (ssDNA) includes functionalizing the ssDNA with amine groups using amine-reactive reagents, including NHS esters or isothiocyanates. The functionalized ssDNA is subsequently conjugated to fluorescence tag and bioluminescent tag modified with complementary functional groups, such as carboxylic acid or NHS ester, through amide bond formation. In an alternative embodiment, thiol groups are introduced onto the ssDNA using thiol-reactive reagents like maleimides or haloacetamides, followed by conjugation to fluorescence tag and bioluminescent tag modified with complementary functional groups, such as maleimide or iodoacetamide, via thioether bond formation. In another embodiment, click chemistry reactions, including azide-alkyne cycloaddition (CuAAC) or strain-promoted azide-alkyne cycloaddition (SPAAC), are utilized to conjugate azide-modified ssDNA and alkyne-modified fluorophores, forming stable triazole linkages. In one embodiment A method for introducing bioluminescent and fluorescence tags to single-stranded DNA (ssDNA) involves first functionalizing the ssDNA with a HaloTag linker. Subsequently, a bioluminescent or fluorescence tag is functionalized with a HaloTag protein. The HaloTag protein-functionalized tag is then conjugated with the HaloTag linker-functionalized ssDNA, thereby introducing the bioluminescent or fluorescence tag to the ssDNA.

### Acceptor Probe Composition

In some aspects, an acceptor probe includes a fluorescence tag for BRET assembly and an affinity tag to bind with the nanoparticles on the nanoparticle array (Plasmo Matrix Array) surface. In some aspects, the acceptor probe includes a nucleotide sequence ranging from about 10 to 100 bases or from about 10 to 25 bases, complementary to the target analyte, forming the core of the third acceptor probe. In another aspect, the acceptor probe includes a fluorescencetag attached to the 5′ end of the probe, which completes the BRET assembly upon binding with the target analyte/donor probe complex. In a further aspect, the fluorescence tag is a fluorophore. In some aspects, the fluorophore is Oregon green and ligand 18. In some other embodiments, the acceptor probe includes a fluorescence tag attached to the 3′ end of the probe, which completes the BRET assembly upon binding with the target analyte/donor probe complex.

Additionally in one embodiment, these fluorophores are used instead of Green Fluorescent Protein (eGFP, YFP, CF), Red Fluorescent Proteins (mCherry, DsRed, mRFP), Fluorescent Dyes (Fluorescein and derivatives, Rhodamine and derivatives, Cy3 and Cy5 dyes), A quantum dot (Semiconductor nanoparticles with size-tunable emission properties, suitable for BRET when matched correctly with the bioluminescent donor) Organic Dyes (Alexa Fluor dyes, DyLight Fluor dyes, BODIPY dyes, Allophycocyanin (APC) and Phycoerythrin (PE), Fluorescent Nanoparticles, or Lanthanide Chelates.

### Affinity Tag on Acceptor Probe

In some aspects, the acceptor probe is immobilized on the nanoparticle array (Plasmo Matrix Array) surface through an affinity tag. The acceptor probe also contains a fluorescence tag and, in the case of nucleic acid detection, contains sequences complementary to the target or extended probe.

In some aspects, the acceptor probe is immobilized on the nanoparticle array surface through an affinity tag and contains a fluorescence tag. Separately, the donor probe contains a bioluminescent tag at its 3′ end and an affinity tag at its 5′ end. For protein detection specifically, the capture antibody contains affinity tags, such as a HaloTag® linker label, and another affinity tag such as biotin attached to a photo-cleavable spacer. The affinity tag is designed to bind with the Universal Analyte Enhancer. In another aspect, the affinity tag is a His-tag, a FLAG-tag, a Glutathione S-transferase (GST) tag, a Maltose Binding Protein (MBP) tag, a Strep-tag, an HA-tag, a Myc-tag, an Avi-tag, a V5-tag, a T7-tag, biotin or its derivatives, Thiols, amines or its derivatives, carboxylic acid and it's dervativea SNAP tag, a CLIP tag, or other appropriate tag. The affinity tag facilitates the binding of the second donor probe to nanomaterials in a universal enhancement solution, enhancing the detection sensitivity and specificity.

Various chemical methods are employed to bind DNA strands to affinity tags such as the His-tag, FLAG-tag, GST-tag, MBP-tag, Strep-tag, HA-tag, Myc-tag, Avi-tag, V5-tag, T7-tag, biotin or its derivatives, SNAP tag, CLIP tag, or other appropriate tags. In some aspects, His-tagged DNA can be immobilized using Immobilized Metal Affinity Chromatography (IMAC), where metal ions such as Ni2+ or Co2+ coordinate with histidine residues in the His-tag. In some aspects FLAG-tagged DNA can be captured using anti-FLAG antibodies immobilized on a solid support. GST-tagged DNA can be bound to glutathione-coated surfaces through a GST affinity interaction. In some aspects, other tags like MBP, Strep-tag, HA-tag, Myc-tag, Avi-tag, V5-tag, and T7-tag can be immobilized using corresponding affinity chromatography methods or specific antibodies. In some aspects, biotinylating can be achieved through methods such as NHS-biotin labeling, DBCO-biotin click chemistry, or maleimide-biotin conjugation. In some aspect amine modification is commonly performed using NHS ester coupling, isocyanate coupling, or EDC/NHS coupling. In some aspects, thiol modification techniques include maleimide-thiol conjugation, haloacetamide-thiol conjugation, and thiol-disulfide exchange. In some aspects affinity group conjugation can be accomplished through carbodiimide coupling, sulfo-SMCC crosslinking, or DCC-mediated coupling. In some aspects, HaloTag ligands to single-stranded DNA/antibodies can be achieved using methods such as amine modification followed by HaloTag ligand conjugation or direct conjugation of HaloTag ligands through NHS ester coupling or other suitable chemistries. In some aspects, we used commonly used technology/chemistries to probe/antibodies to functionalize with the target affinity tag.

In some aspects, the probe binds to the gold surface of a nanoparticle via an Au—S covalent bond. In another aspect, the probe binds to the gold surface of a nanoparticle through linkage mechanisms including but not limited to amide bonds, thiol-ene reactions, click chemistry, electrostatic interactions, dative bonding, physisorption, bidentate or multidentate linkers, hydrophobic interactions, biomolecular conjugation, and layer-by-layer assembly.

### Nanoparticle Array Surface Composition

The nanoparticle array (Plasmo Matrix Array) surface, or Plasmo Matrix Array surface, includes gold nanoparticles arrayed on a substrate, which is 3D or 2D in nature. In one embodiment, the substrate material is a custom nanofiber array as described in U.S. Ser. No. 18/174,463, filed Feb. 24, 2023, herein incorporated by reference in its entirety (see for example FIGS. 21A-21E).

In some aspects, the substrate material is glass, an elastomeric polymer, polydimethylsiloxane (PDMS), ECOFLEX®, SILBIONE®, Indium tin oxide (ITO), or Silicon wafers or semiconductor materials, polyethylene terephthalate (PET), polyurethane (PU), polyethylene naphthalate (PEN), polyimide (PI), polybutadiene, polyisoprene, various silanes, polyamine, polymethylmethacrylate (PMMA), polydopamine, polyvinylpyrrolidone (PVP), polyvinyl alcohol (PVA), (3-aminopropyl)trialkoxysilane, (3-aminopropyl)triaryloxysilane, (3-aminopropyl)triethoxysilane (APTES), (3-aminopropyl)trimethoxysilane (APTMS), trimethoxy(propyl)silane (TMPS), (3-mercaptopropyl)trimethyoxysilane (MPTMS), polyamine, polyolefin, polyamide, polyimide, proteins, silk, cellulose, polyelectrolytes, peptoids, silicon oxide, aluminum oxide, zinc oxide, titanium oxide, graphene, graphene oxide, MoS2, MXenes other substrate materials, or combinations thereof.

### Nanoparticle Variability on Nanoparticle Array Surface

In some aspects, the nanoparticle is primarily a gold spherical particle. In one embodiment, the shape of the nanoparticle is spherical, rod-shaped, triangular, prismatic, cube-shaped, star-shaped, wire-shaped, a sheet, tube-shaped, hollow or cage-like, flower-shaped, disk-shaped, a nanopyramid, a nanobipyramid, a nanoplate, a self-assembled nanostructure, a bowtie antenna, a nano island, a nanoshell, or a combination thereof.

In some aspects, the material of the nanoparticle is gold, silver, iron oxide, a quantum dot, a carbon-based nanoparticle (carbon nanotubes, graphene, fullerenes), a chemical nanoparticle, a liposome, a polymeric nanoparticle, a dendrimer, a magnetic nanoparticle, a silica nanoparticle, a metal oxide nanoparticle, a semiconductor nanoparticle, an MXene nanoparticle, or another possible 1D/2D/3D nanoparticle or combinations thereof. In some aspects, the material of the nanoparticle is gold. In some aspects, the size of the nanoparticle is 4-2000 nm.

In some aspects, there is a spacer between the nanoparticle and the acceptor probe, In another aspect, the spacer between nanoparticle and the acceptor probe has a thickness of from 0.5 to 20 nm. In some aspects, the spacer includes various types of groups or polymers. In another aspect, the spacer includes polyethylene glycol (PEG), polyamino acids, polyacrylamides, polyvinylpyrrolidone, zwitterionic polymers, polysaccharides, poly(N-(2-hydroxypropyl)methacrylamide), poly(oligo(ethylene glycol), methyl ether methacrylate), carboxylic dextran, hydrocarbon chains, substituted hydrocarbon chains, silane groups ((3-Aminopropyl)triethoxysilane (APTES), or 3-mercaptopropyl triethoxysilane (MPTES) other substrate materials, or combinations thereof.

### The Universal Signal Enhancer (USE) Solution

The universal signal enhancer (USE) solution is a solution that contains a nanorod with an affinity tag. The affinity tag binds to the nanorod with or without a spacer. In some aspects, the nanorod is primarily a gold nanoparticle. In some aspects, the nanoparticle has variability in shape. In another aspect, the nanoparticle is spherical, rod-shaped, triangular, prismatic, cube-shaped, star-shaped, wire-shaped, a sheet, tube-shaped, hollow or cage-like, flower-shaped, disk-shaped, a nanopyramid, a nanobipyramid, a nanoplate, a self-assembled nanostructure, a bowtie antenna, a nano island, a nanoshell, or a combination thereof. In some aspects, the material of the nanoparticles includes gold, silver, iron oxide, a quantum dot, a carbon-based nanoparticle (carbon nanotubes, graphene, fullerenes), a chemical nanoparticle, a liposome, a polymeric nanoparticle, a dendrimer, a magnetic nanoparticle, a silica nanoparticle, a metal oxide nanoparticle, a semiconductor nanoparticle, a MXene nanoparticle, or other possible 1D/2D/3D nanoparticle or combinations thereof. In some aspects, the material of the nanoparticle includes gold.

In some aspects, the size of the nanoparticle is 1-100 nm. In some aspects, the nanorod has a length of from about 25 nm to about 2000 nm and a diameter of from about 4 nm to about 100 nm.

In some aspects, the spacer between the nanoparticle and the anti-fluorescent/bioluminescent/chemiluminescent antibody or affinity tag has a thickness of from 0.5 to 20 nm. In some aspects, the spacer includes various types of groups or polymers. In some aspects, the spacer includes polyethyleneglycol (PEG), polyaminoacids, polyacrylamides, polyvinylpyrrolidone, zwitterionic polymers, polysaccharides, poly(N-(2-hydroxypropyl)methacrylamide), poly(oligo(ethylene glycol), methyl ether methacrylate), carboxylic dextran, hydrocarbon chains and substituted hydrocarbon chains, silane group ((3-Aminopropyl)triethoxysilane (APTES)), or 3-mercaptopropyl triethoxysilane (MPTES) other substrate materials, or combinations thereof.

### Substrate Solution

In some aspects, substrate solutions are varied based on the different BRET complex. In some aspects, the solution includes a nanoluc substrate solution with a PBS buffer in 1:100 ratio. In another aspect the ratio varies according to the requirements of the complex.

### Capture Antibody Composition

In some aspects, the method for capturing target proteins or peptides using a specially designed capture antibody includes binding epitopes, an affinity tag with a photocleavable spacer, and another affinity tag to bind with signal enhancer nanoparticle. In some aspects, the capture antibody is equipped with binding epitopes specifically targeting the protein or peptide of interest. In another aspect, the affinity tag is biotin and it binds with the streptavidin functionalized magnetic beads. In another aspect, the affinity tag is not limited to biotin. In some aspects, an affinity tag is attached to the capture antibody to facilitate binding with universal signal enhancer solution nanoparticles.

### Affinity Tag

In some aspects, the affinity tag includes His-tag, FLAG-tag, Glutathione S-transferase (GST) tag, Maltose Binding Protein (MBP) tag, Strep-tag, HA-tag, Myc-tag, Avi-tag, V5-tag, T7-tag, biotin, SNAP tag, CLIP tag, Halo-tag, amine, carboxylic acid, amino acids, thiol (SH), hydroxyl (OH), phosphate group, azide group, alkaline group, ketone group, halide groups, or any functional derivative of any of these affinity tags. In some embodiments, this includes functional derivatives which can facilitate the binding of the magnetic beads with particular binding sites.

### The Photo-Cleavable Spacer

In some aspects, the photo-cleavable spacer is nitrobenzyl, nitrophenyl or their derivatives, coumarin-based linkers or their derivatives, benzoin esters or their derivatives, caged compounds or their derivatives, 2-nitrobenzyl alcohol derivatives, quinone-based spacers or their derivatives, azobenzene derivatives or their derivatives, or caged nucleotides or their derivatives.

### Alternative Cleavage Mechanism

In some aspects, the photo-cleavable spacer is substituted with sequences recognized by restriction endonucleases such as EcoRI, HindIII, or BamHI, accompanied by 6-10 additional base sequences. This allows for the use of restriction endonucleases enzymes to cleave the bond.

### Donor Antibody

In some aspects, the donor antibody has epitope binding sites for the target protein or peptide and also it is functionalized with bioluminescent molecules. In some aspects, the bioluminescent tag is NanoLuc (Engineered Luciferase). In another aspect, the bioluminescent tag is Firefly Luciferase, Renilla Luciferase, Aequorin, Gaussia Luciferase, Bacterial Luciferase, or another bioluminescent tag. Luciferin, Luciferyl adenylate firefly luciferase, Renilla luciferase, aequorin, Gaussia luciferase, or bacterial luciferase, coelenterazin aequorin, dinoflagellate luciferin Photoprotein, nanoluc luciferase, cypridina luciferase, nanobit-smallbit, nanobit-largebit or other suitable bioluminescent tag.

### Acceptor Antibody

In some aspects, the acceptor antibody contains binding sites for the donor antibody and it is functionalized with a fluorescence molecule and with an affinity tag to bind with nanoparticles on the nanoparticle array, which completes the BRET assembly upon binding with the target analyte/donor antibody complex. In some aspects, the fluorophore is Oregon green and ligand 18. In another aspect, the fluorophore green fluorescent protein (eGFP, YFP, CF), red fluorescent proteins (mCherry, DsRed, mRFP), fluorescent dyes (fluorescein and derivatives, rhodamine and derivatives, Cy3 and Cy5 dyes), a quantum dot (semiconductor nanoparticles with size-tunable emission properties, suitable for BRET when matched correctly with the bioluminescent donor), organic dyes (Alexa Fluor dyes, DyLight Fluor dyes, BODIPY dyes, Allophycocyanin (APC) and Phycoerythrin (PE), Fluorescent Nanoparticles, or Lanthanide Chelates.

### Affinity Tag on Acceptor Antibody

In some aspects, an acceptor probe is immobilized on the nanoparticle array (Plasmo Matrix Array) surface through an affinity tag. In another aspect, the antibody binds to the gold surface of the nanoparticles via an Au—S covalent bond with or without a spacer, or alternatively through linkage mechanisms including but not limited to amide bonds, thiol-ene reactions, click chemistry, electrostatic interactions, dative bonding, physisorption, bidentate or multidentate linkers, hydrophobic interactions, biomolecular conjugation, and layer-by-layer assembly.

### Additional Disclosure

In the described application, Bioluminescent Resonance Energy Transfer (BRET) plays an important role, where the donor probe incorporates bioluminescent capabilities, and the acceptor probe, attached to the nanoparticle array (Plasmo Matrix Array), is tagged by a fluorescence marker. This setup facilitates a remarkable interaction: upon the donor probe's binding with the target analyte, a BRET donor-acceptor complex is formed.

For the detection system to produce the desired signal, the bioluminescent tag on the donor probe and the fluorophore tag on the acceptor probe are positioned at a distance less than 10 nanometers (nm) from each other upon the completion of hybridization. Accordingly, complementary sequences are designed in close proximity to the target analyte. These sequences are crafted with a length variation ranging from 0 to 30 nucleotides (nts) to achieve optimal proximity. The complementary sequences are tailored to facilitate maximum BRET formation, maintaining a minimum distance of 10 nm between the bioluminescent and fluorophore tags. These distances are applicable for the protein assay as well.

In some aspects, this assembly is constructed in a reverse configuration, wherein the acceptor probe initially binds to the analyte, and the donor probe is immobilized on the nanoparticle array (Plasmo Matrix Array). In this assembly, the fluorescence signal is predominantly used to achieve high specificity. Additionally, the ratio of bioluminescent to fluorescence is employed as an analytical metric.

The disclosed MIP assay design offers the flexibility to employ Fluorescence Resonance Energy Transfer (FRET) as an alternative to the BRET system. In this approach, the donor probe is equipped with a donor fluorophore, while the acceptor probe carries a corresponding acceptor fluorophore. In some aspects, the arrangement of these probes is varied: the donor probe either follows the capture probe in a solution-based approach or is immobilized onto the nanoparticle array (Plasmo Matrix Array). Similarly, in some aspects, the acceptor probe is either immobilized onto the nanoparticle array (Plasmo Matrix Array) or introduced after the capture probe in solution, as previously described.

Furthermore, this versatile system is not limited to energy transfer mechanisms alone. In one embodiment, it is adapted to function with individual luminescence technologies such as bioluminescent, fluorescence, phosphorescence, or chemiluminescence. In such configurations, the donor probe is associated with one of these luminescent methods, without the acceptor probe being functionalized with a dye. This modification means that the detection of the target analyte is signaled solely by emission from the chosen luminescence method, which in some aspects is bioluminescent, fluorescence, phosphorescence, chemiluminescence, or other similar technologies. This approach enhances the MIP assay's adaptability, enabling its application across a broad spectrum of luminescence-based detection scenarios.

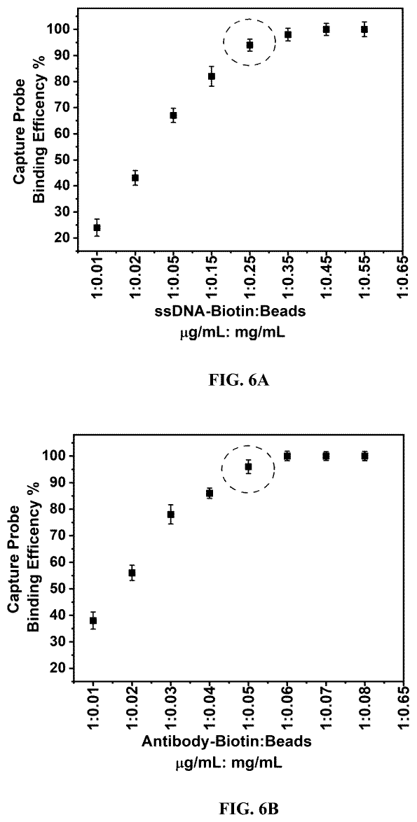

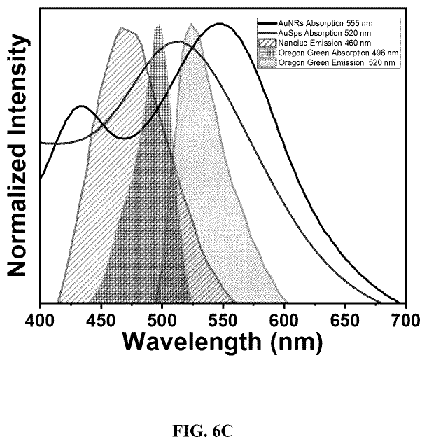

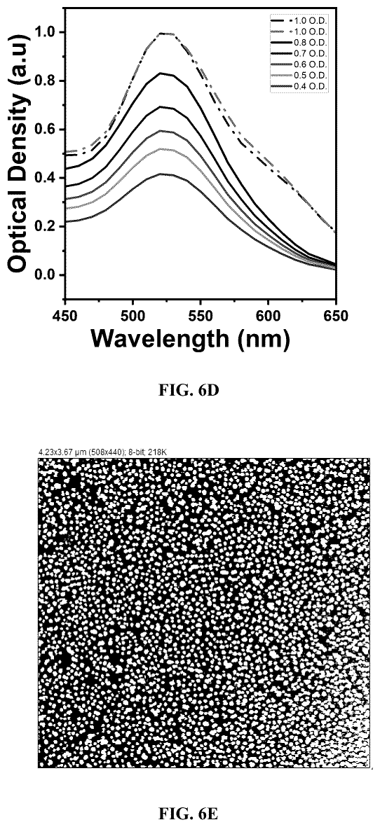

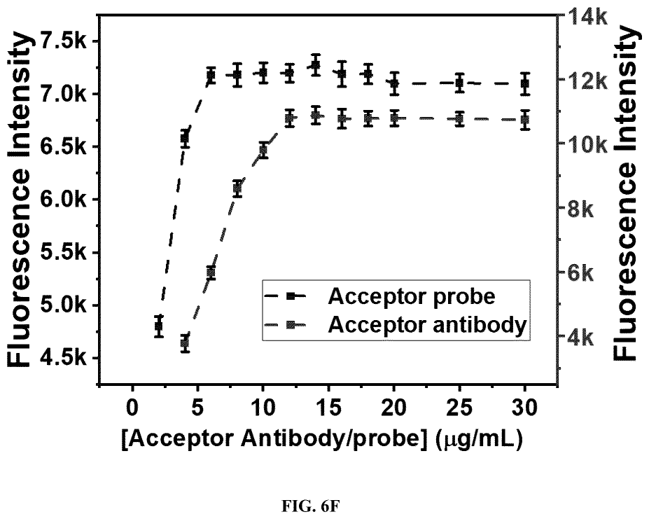

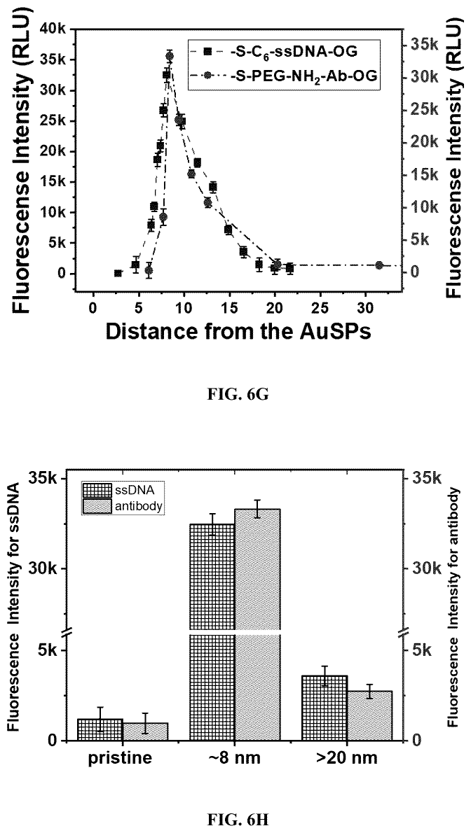

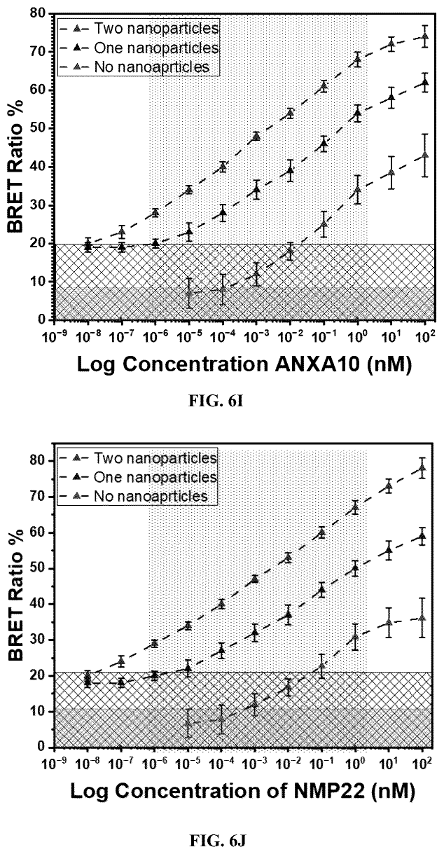

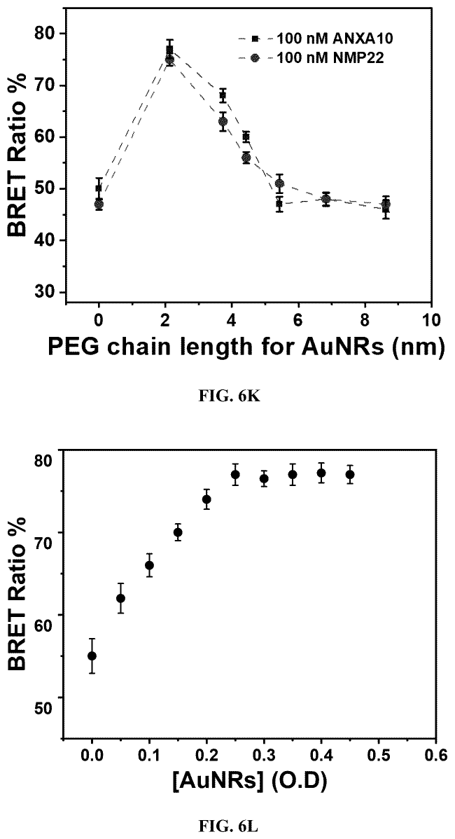

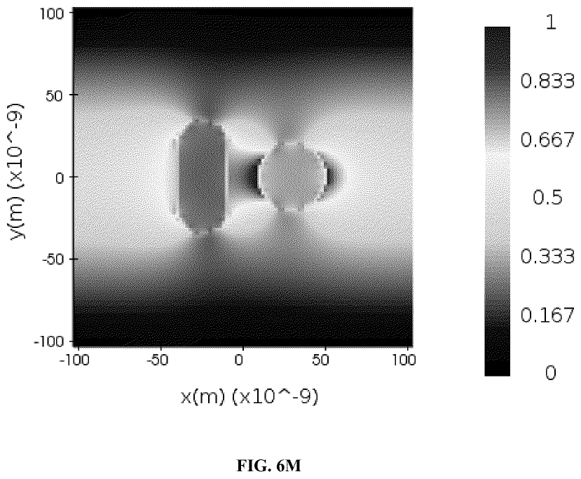

The design of the application is versatile, allowing for the capture of analytes from either the 3′ to 5′ end or vice versa, from the 5′ to 3′ end. This adaptability means that depending on the direction of analyte capture chosen, the specified ends on the capture probe, donor probe, and acceptor probe can be altered accordingly to their opposite orientations. This flexibility in the assay setup ensures that in some aspects, the MIP assay is tailored to target and capture the analyte effectively, regardless of the orientation of the analyte's sequence, enhancing the application's utility across various experimental conditions and requirements. See for example, U.S. patent application Ser. No. 18/174,463, filed Feb. 24, 2023 herein incorporated by reference in its entirety. See for example FIGS. 6A-FIG. 6B, pages 37-38, [000191].

The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Development of a Multi-Omic Integration Platform (MIP) for Quantification

This example describes the development of a Multi-Omic Integration Platform (MIP) for the quantification of low-abundance analytes, addressing limitations of standard immunoassay techniques.

Standard immunoassay techniques like ELISA often struggle to detect target analytes at very low concentrations due to limited sensitivity. Conventional immunoassays typically use small sample volumes (100-200 μL), which restricts their ability to identify low-abundance molecules. For instance, detecting analytes at 100 attomolar (aM) concentrations (approximately 10,000 RNA copies or 6,000 protein molecules) in sample volumes of 0.1 to 1 mL is challenging. While external extraction techniques can concentrate RNAs and proteins from larger sample volumes, the ultimate recovery of the analyte significantly influences sensitivity and accuracy.

Furthermore, the ‘Monte Carlo’ effect observed in quantitative PCR (qPCR) and NGS sequencing complicates the detection of low-abundance targets, introducing variability and randomness in the amplification process, especially with low template copy numbers. This variability can result in inconsistent outcomes and hinder the accuracy of gene quantification. Similarly, the detection and quantification of proteins are challenged by stochastic effects in biochemical reactions, issues with non-specific binding, protein stability and degradation, variability in antibodies, and matrix effects.

To address these challenges, a dual-probe approach was implemented to capture and label the target analyte. The beads were then removed, allowing the captured and labeled analytes to be introduced to a platform for a third specific binding interaction. By utilizing plasmonic nanostructures, the BRET (bioluminescent and fluorescence) signal was significantly amplified beyond the background noise, enabling the detection of low-abundance molecules.

The focus was on identifying the essential parameters: concentrations of capture probes and beads, levels of labeling reagents (donor and acceptor probes), and the distances and wavelength compatibility between plasmonic nanostructures and the absorbance/emission peaks of the donor and acceptor tags. This optimization was crucial to ensuring the assay's sensitivity and accuracy in detecting low-abundance molecules. A summary of the MIP design and the optimized parameter conditions is provided in FIG. 5.

These results demonstrate that the developed Multi-Omic Integration Platform (MIP) can overcome the limitations of standard immunoassay techniques by employing a dual-probe approach, plasmonic nanostructures, and optimized assay parameters. This approach enables the detection and quantification of low-abundance analytes with improved sensitivity and accuracy compared to conventional methods.

### Example 1.1

### Capture Probe/Antibody Concentration

This example describes the optimization of capture probe and antibody concentrations for the Multi-Omic Integration Platform (MIP) technology, addressing limitations in standard immunoassays for detecting low-abundance analytes.

The concentration of capture probes is crucial for effectively capturing target analytes in biospecimens. Standard immunoassays are often limited by mass transport from solution to surface, as described by Langmuir binding isotherm theory. This is particularly challenging when analyte quantities are minimal, as achieving equilibrium through diffusion can be time-consuming. The MIP technology addresses this by positioning the capture probe closer to the analyte within the biospecimen, circumventing thermodynamic and kinetic restrictions and minimizing steric hindrance.

For nucleic acid capture, a solution containing 1 μg/mL (125 pmol/mL) of ssDNA capture probes (molecular weight ˜8 kDa) results in approximately 7.53×10¹³ molecules per mL. This creates an average distance of about 240 nm between capture probes in a 1 mL solution, ensuring effective binding within two hours. Even for an analyte with 400 nucleotides at an extremely low concentration of 1.3×10⁻¹¹ μg/mL (100 aM), the average distance between analytes is about 0.2 mm, allowing for rapid interactions with capture probes in under a minute.

Similarly, for protein capture, a 10 μg/mL solution of capture antibodies (average size 150 kDa) contains about 4.01×10¹³ antibody molecules per mL, with an average distance of 292 nm between antibodies. For proteins at low concentrations (3×10⁻⁹ μg/mL or 100 aM), regardless of size, they are spaced approximately 255 μm apart, with about 900 antibodies between each analyte. This arrangement facilitates rapid interactions between proteins and capture antibodies in under one minute.

The chosen concentrations of ssDNA (1 μg/mL) and antibodies (10 μg/mL) provide an ample supply of capture probes or antibodies between analytes, even at low concentrations, ensuring highly efficient binding. Optimization of the capture probe ratio is essential for downstream applications, helping to avoid excessive probes and preventing overcrowding of target analytes, thus ensuring effective binding with the acceptor probe on the nanoparticle array.

These results demonstrate that the MIP technology's approach to capture probe and antibody concentration optimization overcomes limitations in standard immunoassays, enabling efficient capture and detection of low-abundance analytes. This optimization is crucial for the platform's ability to detect and quantify analytes at extremely low concentrations with improved sensitivity and accuracy.

### Example 1.2

### Magnetic Bead Concentration

This example describes the optimization of magnetic bead concentrations for capturing nucleic acids and proteins in the Multi-Omic Integration Platform (MIP) technology.

Each streptavidin bead (1 μm diameter, volume 5.24×10¹⁹ m³, density 1.6 g/cm³) can bind approximately 253,000 biotinylated ssDNA molecules or 67,500 biotinylated IgG molecules. One milligram of beads contains roughly 1.19×10⁹ beads. To bind 7.53×10¹³ ssDNA molecules, 297,628 beads (0.25 mg/mL) are required, while 59,556 beads (0.05 mg/mL) are needed for 4.02×10¹² antibody molecules. These concentrations result in theoretical ratios of 1:253,000 (beads to ssDNA) and 1:67,500 (beads to antibodies) respectively.

To identify optimal bead concentrations, preliminary experiments were conducted. For ssDNA capture, bead concentrations from 0.01 mg/mL to 0.55 mg/mL were evaluated with 1 μg/mL ssDNA. For antibody capture, bead concentrations from 0.01 mg/mL to 0.08 mg/mL were tested with 10 μg/mL antibodies. Capture probes and antibodies were labeled with Oregon Green (OG) fluorescence, and capturing efficiency was determined by comparing initial and post-separation fluorescence intensities.

The results showed highest binding efficiency at 0.25 mg/mL beads for 1 μg/mL ssDNA and 0.05 mg/mL beads for 10 μg/mL antibodies. These findings are consistent with theoretical predictions and manufacturer protocols, as illustrated in FIG. 6A and FIG. 6B.

FIG. 6A shows capturing efficiencies for various bead to ssDNA ratios, with the optimal concentration at 0.25 mg/mL beads per 1 μg/mL ssDNA. FIG. 6B displays capturing efficiencies for bead to antibody ratios, with highest efficiency at 0.05 mg/mL beads per 10 μg/mL antibodies.

These results demonstrate that the optimized magnetic bead concentrations in the MIP technology enable efficient capture of both nucleic acids and proteins. The experimental findings align well with theoretical predictions and published guidelines, confirming the effectiveness of specific bead concentrations for optimal binding in various biological assays. This optimization is crucial for the platform's ability to capture low-abundance analytes with high efficiency, contributing to the overall sensitivity and accuracy of the MIP technology.

### Example 1.3

### Donor Probe/Antibody Concentration

This example describes the optimization of donor probe and antibody concentrations for the Multi-Omic Integration Platform (MIP) technology.

To optimize the donor probe concentration, the same capture probe-to-analyte ratio was maintained, aiming for maximum binding efficiency between the analyte and the donor probe. A concentration of 1 μg/mL for the donor probe and 10 μg/mL for the donor antibody was employed. This approach ensured that there were enough donor probes to hybridize with all target analytes.

The use of magnetic separation allowed for the elimination of any excess donor probes, ensuring they did not interfere with subsequent applications. This step is crucial for maintaining the specificity and sensitivity of the assay.

These results demonstrate that the optimized donor probe and antibody concentrations in the MIP technology enable efficient labeling of captured analytes while minimizing background interference. This optimization contributes to the overall sensitivity and accuracy of the platform, allowing for effective detection and quantification of low-abundance analytes. The ability to remove excess probes through magnetic separation further enhances the specificity of the assay, reducing potential false positives in downstream applications.

### Example 1.4

### Selection and Concentration of AuSPs for the Nanoparticle Array

This example describes the selection and optimization of gold spherical nanoparticles (AuSP) concentration for the nanoparticle array surface in the Multi-Omic Integration Platform (MIP) technology.

Gold spherical nanoparticles (AuSP) were chosen for the nanoparticle array surface due to their advantageous optical characteristics. With a plasmon peak at 520 nm and a scattering profile extending from 410 to 700 nm, AuSPs show considerable overlap with both the NanoLuc bioluminescent emission (peak at 465 nm) and Oregon Green (OG) excitation (peak at 498 nm) and emission (peak at 526 nm). This overlap is illustrated in FIG. 6C, which shows the absorption scattering profiles of AuSPs and AuNRs.

Theoretical analyses indicate that a single half-well with a 5 mm diameter can accommodate approximately 111 billion AuSPs in a monolayer. This greatly enhances the available surface area for binding the acceptor probe, representing a 20-fold increase compared to a standard half-well flat-bottom immunoassay.

To optimize the concentration of AuSPs for maximum surface coverage, wells were prepared using gold nanoparticle solutions with different optical densities (OD). Experiments demonstrated that an OD of 3.0 produced the highest UV absorption peaks (0.80) without leading to nanoparticle aggregation, as shown in FIG. 6D.

These results were confirmed through scanning electron microscopy (SEM) imaging, which showed that the 3.0 OD AuSP solution achieved approximately 60% coverage of a single monolayer of AuSPs on the surface (FIG. 6E).

These results demonstrate that the optimized concentration of AuSPs (3.0 OD) allows for the greatest possible surface coverage while ensuring the stability and uniformity of the nanoparticle layer. This optimization is essential for the effectiveness of the MIP technology, as it maximizes the available surface area for binding acceptor probes while maintaining the optical properties necessary for signal amplification. The increased surface area and optimal nanoparticle distribution contribute to the platform's enhanced sensitivity in detecting low-abundance analytes.

### Example 1.5

### Acceptor Probe/Antibody Concentration

This example describes the optimization of acceptor probe and antibody concentrations for the Multi-Omic Integration Platform (MIP) technology, as well as the determination of optimal distances for fluorescence enhancement.

In theory, achieving complete coverage of the 15 nm AuSP layer would necessitate around 9.4×10¹³ thiolate ssDNA-SH molecules (acceptor probe), with each molecule occupying an area of 0.25 nm². However, considering the presence of PEG spacers on the AuSPs, it is anticipated that approximately 4.7×10¹³ acceptor probes can be achieved on the AuSP layer. To attain this coverage, a solution of ssDNA at a concentration of 10 μg/mL is required, which yields about 1.5×10¹⁴ molecules (with a molecular weight of 8 kDa in 100 μL). For antibodies linked to NH2-PEG-SH, a comparable binding quantity (4.7×10¹³) on the AuSP layer is also expected due to the same thiolate footprint. An antibody solution concentration of 20 μg/mL should provide around 1.61×10¹³ molecules (˜150 kDa in 200 μL), which is adequate for full coverage of the AuSP layer. Ultimately, this setup will yield approximately 1.58×10¹¹ molecules/cm² on the enhanced surface area created by the 15 nm AuSPs.

Through experiments, the optimal concentrations of the acceptor probe (SH-ssDNA-OG) and acceptor antibodies for achieving maximum coverage were identified. In this investigation, a range of 0-30 μg/mL of SH-ssDNA-OG with an OG tag was utilized, maintaining an approximate distance of 15 nm (40 nts). FIG. 6F illustrates a graph showing the determination of optimal (acceptor probe/antibody) ssDNA-SH and antibody concentrations for enhanced fluorescence. Concentrations ranging from 1 to 30 μg/mL were tested for both OG-ssDNA-SH (40 nts) and antibodies OG-PEG-1K-SH to identify the conditions yielding maximum fluorescence enhancement. The optimal concentration was found to be 6 μg/mL for ssDNA-SH and 12 μg/mL for antibodies. The results indicated that a concentration of 6 μg/mL yielded the highest fluorescence intensity, signifying that this concentration is adequate for achieving maximum coverage (FIG. 6F, blue square), which is lower than predicted by theoretical calculations. Likewise, experiments with antibodies determined that a concentration of 12 μg/mL provided optimal coverage and fluorescence intensity, aligning closely with theoretical predictions. For this study, —NH2-PEG-1000-SH with a length of about 8.6 nm was employed, while the average length of the antibody is around 3-4 nm, resulting in approximately 12.6 nm from the nanoparticle (FIG. 6F, red circle).

Plasmonic nanostructures induce a substantially amplified electromagnetic field in their immediate vicinity, resulting in a notable distance-dependent fluorescence enhancement. When fluorophores are directly adjacent to or in direct contact with these nanostructures, nonradiative energy transfer to the metal surface leads to fluorescence quenching. In contrast, as the separation between fluorophores and nanostructures increases, the fluorescence enhancement diminishes due to the attenuation of the electromagnetic field. Thus, achieving an optimal distance between the metal surface and the fluorophores is critical for maximizing fluorescence enhancement.

To identify the optimal distance from the nanoparticle array surface for maximizing fluorescence enhancement on the acceptor probe, a series of controlled experiments were conducted. These experiments involved adjusting the lengths of nucleotides (nts) for nucleic acid (NA) applications using SH-C6-ssDNA-OG, as well as testing different molecular weights (MW) of polyethylene glycol (PEG) chains for protein applications with OG-Ab-NH2-PEG-SH. Comprehensive calculations regarding nucleotide quantities, estimated distances from the nanoparticles, the MW of NH2-PEG-SH, and approximate distances from the gold surface plasmon (AuSP) are detailed in Table 1.

FIG. 6G illustrates a graph showing distance-dependent fluorescence enhancement by plasmonic nanostructures. Optimal distances from gold nanoparticles are 20 nt (˜8 nm, blue square) for nucleic acids and ˜500 g/mol PEG (˜4.4 nm, red circle) for antibodies. The findings indicated that the highest fluorescence enhancement occurred at a distance of 20 nts from the AuSP surface (FIG. 6G, blue square). This setup, featuring a C6 spacer and a C—S—Au bond, achieves an estimated separation of approximately 8 nm from the AuSP. In the case of antibody applications, optimal enhancement was observed with a PEG chain of around 500 g/mol (PEG12), which resulted in a thickness of roughly 4.4 nm. However, the total distance from the AuSPs may also account for the inherent length of the antibody, estimated to be between 3 and 4 nm (from the end of the Fc region to the NH2 binding sites) (FIG. 6G, Red circle).

TABLE 1
Length (nm) measurements from the AuSP surface to the acceptor
tag; -S-C6-ssDNA-OG and -S-PEG-NH2-Antibody (Ab)-OG

| -S-C6-ssDNA-OG | | | -S-PEG-NH2-Ab-OG | | |
|---|---|---|---|---|---|
| | | Length | | | Length |
| | | from | | | from |
| | | the | | | the |
| | | Nano- | | | nano- |
| | | particle | PEG- | | particle |
| | | to OG | molecular | PEG- | to OG |
| Number of | Nucleotides | (Acceptor | weight | length | (Acceptor |
| Nucleotides | Length (nm) | Tag) | (g/mol) | (nm) | Tag) |
| 5 | 1.7 | 3 | 200 | 2.1 | 6.1 |
| 10 | 3.4 | 4.7 | 400 | 3.7 | 7.7 |
| 15 | 5.1 | 6.4 | 500 | 4.4 | 8.4 |
| 16 | 5.4 | 6.7 | 600 | 5.4 | 9.4 |
| 17 | 5.8 | 7.1 | 800 | 6.8 | 10.8 |
| 18 | 6.1 | 7.4 | 1000 | 8.6 | 12.6 |
| 19 | 6.5 | 7.7 | 2000 | 16.3 | 20.3 |
| 20 | 6.8 | 8.1 | 3400 | 27.5 | 31.5 |
| 25 | 8.5 | 9.8 | 4000 | 32.3 | 36.3 |
| 30 | 10.2 | 11.5 | | | |
| 35 | 11.9 | 13.2 | | | |
| 40 | 13.6 | 14.9 | | | |
| 45 | 15.3 | 16.6 | | | |
| 50 | 17 | 18.3 | | | |
| 55 | 18.7 | 20 | | | |
| 60 | 20.4 | 21.7 | | | |

Using these configurations, the fluorescence enhancement of 20 nts 6 μg/mL SH-C6-ssDNA-OG and 500 g/mol 12 μg/mL Ab-NH2-PEG-SH MW on AuSPs was compared with that of standard immunoassay functionalization of 6 μg/mL SH-C6-ssDNA-OG and 12 μg/mL Ab on APTES-functionalized, unenhanced (pristine) wells. FIG. 6H illustrates a bar graph showing fluorescence enhancement. Fluorescence enhancement is compared between samples without nanoparticles (pristine surface) and samples at approximately 8 nm and greater than 20 nm distances from the AuSP nanoparticle array surface. The first two columns represent the pristine surface, where the blue cross bars correspond to 6 μg/mL —S-C6-ssDNA-OG (20 nucleotides, approximately 8 nm in length) and the red waves represent 12 μg/mL Ab-NH₂—PEG-S— (500 g/mol, approximately 8 nm). The second two columns show these same samples positioned at approximately 8 nm from the AuSP surface. The third set of columns shows the AuSP surface with greater than 20 nm: the blue cross bars indicate 6 μg/mL SH-C6-ssDNA-OG (60 nucleotides, greater than 20 nm in length), and the red wavy bars represent 12 μg/mL Ab-NH2-PEG-SH (2000 g/mol, greater than 20 nm). This demonstrates the effect of varying distances from the AuSP surface on fluorescence enhancement. The measurements showed approximately ˜30-fold increased fluorescence compared to the pristine conditions (FIG. 611). This significant enhancement is attributed to two main factors: the electromagnetic (EM) field enhancement facilitated by the plasmonic nanostructures at the optimal distance, and the sustained high fluorescence even beyond the 20 nm enhancement region, which is still superior to that of the pristine conditions. This enhancement is likely further supported by the incorporation of gold nanoparticles (AuNPs), which increases the surface area, thus boosting the fluorescence signal even without direct EM field enhancement.

In BRET assays, it is crucial for the distance between the bioluminescent and fluorescent entities to be less than 10 nm to ensure efficient energy transfer. A closer proximity between these entities leads to more effective BRET coupling. Therefore, for the nucleic acid (NA), a constant distance of 5-20 nucleotides between the acceptor binding region and the donor probe binding region was meticulously maintained. This configuration creates an approximate gap of 3-7 nm between the donor's bioluminescent and the acceptor's fluorescence. Similarly, in the case of the antibody used, the distance between the epitope binding sites (Fab region) and the FC region of the donor antibody create less than ˜4 nm, facilitating an efficient BRET coupling between NanoLuc and OG. Maintaining these specific distances is essential in this application to avoid the crowding effect, which could interfere with the analyte binding to the target acceptor probe, especially in NA application.

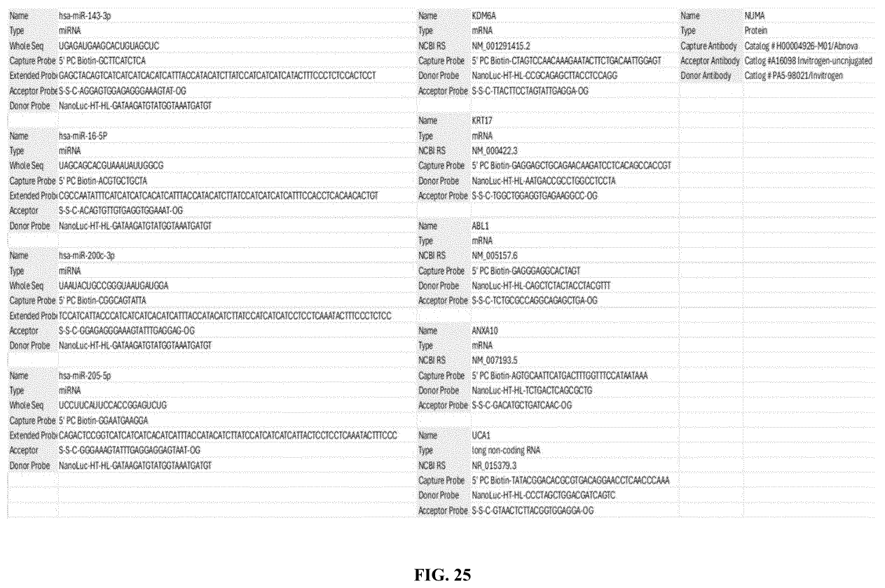

Having optimized the critical parameters for the MIP technology, the implementation of the BRET assay on the MIP platform proceeded. The objective was to achieve LSPR enhanced BRET by maintaining optimal spacing between the BRET pair (comprising bioluminescent and fluorescence molecules) and the metal surface, as previously discussed. Importantly, prior studies have highlighted the need to maximize the overlap between the LSPR of the nanostructures and the optical emission of the fluorescence/bioluminescent for maximal enhancement. The AuSP nanoparticles, with a plasmon peak of 520 nm and a scattering profile ranging from 410-700 nm with a large FWHM, were chosen to optimize the overlap with the NanoLuc (bioluminescent) emission peak at 465 nm and the Oregon Green (OG) (fluorescence) with an excitation peak at 498 nm and an emission peak at 526 nm (FIG. 6C). Detection of mRNA ANXA10 and protein NMP22 was then performed using optimized probe and antibodies sequences (FIG. 25), along with optimized bead ratios and concentrations. The BRET ratios were detected at concentrations of 100 nM to 100 aM. Repeating the experiment with a pristine, unenhanced setup without nanoparticles, a ˜100-fold enhancement in BRET ratios using AuSPs on the nanoparticle array was observed in FIGS. 6I and 6J. FIG. 6I illustrates a graph showing the development of a standard curve for ANXA10 mRNA. The standard curve for ANXA10 mRNA under three conditions-no nanoparticles, one nanoparticle, and two nanoparticles at 100 nM-100 pM developed. Conditions include no enhancement (purple line), BRET enhancement with one type of nanoparticle (AuSPs, blue line), and BRET enhancement with two types of nanoparticles (AuNRs and AuSPs, red line). FIG. 6J illustrates a graph showing the development of a standard curve for NMP22 protein. The standard curve for ANXA10 mRNA under three conditions-no nanoparticles, one nanoparticle, and two nanoparticles at 100 nM-100 pM developed. Conditions include no enhancement (purple line), BRET enhancement with one type of nanoparticle (AuSPs, blue line), and BRET enhancement with two types of nanoparticles (AuNRs and AuSPs, red line). However, the 100 aM concentration, which is essential for detecting low abundance molecules in multiomic applications, still could not be detected. These results demonstrate that the optimized acceptor probe and antibody concentrations, along with carefully determined distances from the nanoparticle surface, significantly enhance the fluorescence and BRET signals in the MIP technology. The improvements highlight the importance of these optimizations for improving the platform's sensitivity, particularly for detecting low-abundance molecules in multiomic applications.

### Example 1.6

### Selection and Optimization of the Universal Signal Enhancer (USE)

This example describes the selection and optimization of the Universal Signal Enhancer (USE) for the Multi-Omic Integration Platform (MIP) technology, focusing on improving the bioluminescent resonance energy transfer (BRET) system.

To enhance the signal-to-noise ratio and achieve maximum sensitivity in the assay, the focus was on improving the bioluminescent resonance energy transfer (BRET) system. The strategy involved positioning the BRET pair at the plasmonic coupling hotspots of two nanoparticles to further amplify the fluorescence signal. The BRET ratio, defined as the intensity of the acceptor divided by that of the donor, benefits from increased fluorescence through LSPR coupling. Gold nanorods (AuNRs) were chosen due to their tunable plasmon resonances, which allow for precise alignment with the emission or absorption peaks of the donor or acceptor, thereby enhancing BRET efficiency. In contrast, spherical nanoparticles exhibit a single plasmonic peak with limited tunability. The combination of nanorods and spherical nanoparticles can offer a wider range of plasmonic properties and spatial orientations, potentially increasing the overlap in the energy transfer spectrum and enhancing the overall BRET effectiveness. Gold nanorods with a broader scattering profile (400 nm-700 nm) and longitudinal peak at 555 nm and Transverse peak at 500 nm and 25 nm edge length were specifically selected, which synergistically enhance the fluorescence intensity of the OG absorption at 495 nm. This careful selection and the overlapping of the LSPR peak and emission peak is a well-studied area, and the chosen NanoLuc-OG pair with 520 nm AuSP and 550 nm AuNRs provided the optimal configuration for LSPR-enhanced BRET (FIG. 6C).

There are two main approaches for using the Universal Signal Enhancer (USE) solution containing AuNRs to bind with the MIP assay after hybridization with the capture analyte donor complex to the acceptor on the nanoparticle array surface.

### Method 1:

The AuNRs of the USE solution contain AuSP-PEG-NH2-Ab, an antibody designed to bind with various luminescence and fluorescence tags. This antibody serves as an anti-antibody for different tagging applications, such as Anti-NanoLuc for NanoLuc luciferase, Anti-CY3 for fluorescence CY3 tags, and Anti-HRP for HRP chemiluminescence, among others. The PEG spacer provides an optimal distance between the nanoparticles and the tag, enhancing the signal intensity when the anti-luminescence or anti-fluorescence antibody binds to the donor probe tagged with bioluminescent or fluorescence. This configuration significantly boosts the BRET, FRET, fluorescence, bioluminescent, and chemiluminescence intensities, as documented in FIGS. 1.1-1.3, and FIGS. 2A-2E. FIG. 1 is a schematic diagram illustrating the application of one embodiment of the Molecular Identification Platform (MIP) for detecting an analyte from a biospecimen sample based on Method 1. The schematic depicts the MIP assay for various analytes: 1.1a illustrates RNA analytes hybridize with a capture probe. 1.2a illustrates small RNA analytes hybridize with both a capture probe and an extended probe. 1.3a illustrates protein analytes conjugate with capture antibodies, all of which possess affinity tags incorporating a photo-cleavable spacer for magnetic bead binding. 1.1b, 1.2b, and 1.3b illustrate that the analyte-capture complex is isolated using a magnetic bead. 1.1c, 1.2c, and 1.3c illustrate that the isolated analyte-capture complex is then exposed to donor probes or donor antibodies bearing a bioluminescent tag. 1.1d, 1.2d, and 1.3d illustrate that a second magnetic separation step isolates the resulting analyte-capture-donor complex. 1.1e, 1.2e, and 1.3e illustrate that exposure to UV light facilitates the removal of magnetic particles from the complex. 1.1f, 1.2f, and 1.3f illustrate that the analyte-capture-donor solution is subsequently transferred to the Nanoparticle Array Surface, where it forms a Bioluminescent Resonance Energy Transfer (BRET) complex with the acceptor probe or antibody. The acceptor probe or antibody, bearing a fluorescence tag, is immobilized on the nanoparticle array surface. 1.1g, 1.2g, and 1.3g illustrate that a universal signal enhancer solution is added, comprising a metallic nanoparticle with an anti-bioluminescent antibody attached via a spacer. 1.1h, 1.2h, and 1.3h illustrate that a single-addition enzyme substrate reagent is introduced, which emits a glow-type signal for detection and quantification of the analyte. FIG. 2A-2E are schematic diagrams illustrating the Multi-Omic Integration Platform (MIP) assay based on Method 1 for detecting polynucleotides, small RNA, and proteins. FIG. 2A depicts a schematic diagram of the MIP assay platform, wherein acceptor probes or antibodies are immobilized on the nanoparticle array surface. FIG. 2B illustrates a schematic diagram of the MIP assay configured for the detection of nucleic acids, including but not limited to DNA, mRNA, and long non-coding RNA (lncRNA). FIG. 2C presents a schematic diagram of the MIP assay specifically configured for the detection of small nucleic acids, such as small RNA. FIG. 2D shows a schematic diagram of the MIP assay configured for the detection of target proteins. FIG. 2E provides a schematic diagram detailing the components utilized in the MIP assay method 1. This method employs a universal signal enhancer solution containing nanoparticles with an anti-bioluminescent antibody. This antibody is designed to directly bind to the bioluminescent tag present on the donor probe or antibody.

### Method 2:

The 5′ end of the donor probe for nucleic acids features an affinity tag designed to directly interact with the affinity tag on the AuNRs in the USE solution. This setup maintains the optimal distance between the AuNRs and the donor probe, mirroring the spacing method used in the initial approach, utilizing specific nucleotide bases of the donor probe. Once hybridization between the capture analyte and the donor and acceptor complex is complete, the AuNRs from the USE solution bind to the affinity tag of the donor probe, significantly enhancing the BRET, FRET, bioluminescent, fluorescence and chemiluminescence signals, as described in FIGS. 3.1-3.3, and FIGS. 4A-4E. FIG. 3 is a schematic diagram illustrating the application of one embodiment of the Molecular Identification Platform (MIP) to detect an analyte from a biospecimen sample based on Method 2. 3.1 depicts the MIP assay for various analytes: 3.1a illustrates that RNA analytes hybridize with a capture probe. 3.2a illustrates that small RNA analytes hybridize with the capture probe and an extended probe. 3.3a illustrates that protein analytes conjugate with capture antibodies, all of which have affinity tags with a photo-cleavable spacer for magnetic bead binding. 3.1b, 3.2b, and 3.3b illustrate that the analyte-capture complex is separated using a magnetic bead. 3.1c, 3.2c, and 3.3c illustrate that the analyte-capture complex is introduced to donor probes or donor antibodies with an affinity tag on one end and a bioluminescent tag on the other end. 3.1d, 3.2d, and 3.3d illustrate that a second magnetic separation isolates the analyte-capture-donor complex. 3.1e, 3.2e, and 3.3e illustrate that UV exposure facilitates the removal of magnetic particles from the complex. 3.1f, 3.2f, and 3.3f illustrate that the analyte-capture-donor solution is then transferred to the nanoparticle array surface, forming a BRET complex with the acceptor probe or antibody immobilized on the nanoparticle array surface with a fluorescence tag. 3.1g, 3.2g, and 3.3g illustrate that a universal signal enhancer solution, consisting of a metallic nanoparticle with binding sites for the affinity tag of the donor probe or capture antibody, is added. 3.1h, 3.2h, and 3.3h illustrate that a single-addition enzyme substrate reagent is introduced, which emits a glow-type signal for detection and quantification of the analyte. FIGS. 4A-4E are schematic diagrams of the MIP assay based on Method 2 for detecting Polynucleotide, small RNA and protein. FIG. 4A illustrates a schematic diagram of the MIP assay platform, where acceptor probes or antibodies are immobilized on the nanoparticle array surface. FIG. 4B illustrates a schematic diagram of the MIP assay configured to detect nucleic acids such as DNA, mRNA, or long non-coding RNA (lncRNA). FIG. 4C illustrates a schematic diagram of the MIP assay configured for detecting small nucleic acids, such as small RNA. FIG. 4D illustrates a schematic diagram of the MIP assay configured for detecting a target protein. FIG. 4E illustrates a schematic diagram of the components used in the MIP assay method 2, which uses a universal signal enhancer solution containing nanoparticles with an affinity tag that directly binds to the affinity tag on the donor probe or capture antibody.

For proteins, the capture antibody includes an affinity tag that binds with the AuNRs of the USE solution. Upon forming the capture-protein-donor complex, this conjugate interacts with the acceptor antibody on the nanoparticle array, establishing the optimal distance from the BRET, FRET, bioluminescent, and fluorescence tags. This arrangement substantially enhances the signal across these luminescence and fluorescence detection methods.

An experiment was conducted to determine the optimal distance between gold nanorods (AuNRs) from the BRET pair for detecting ANXA10 and NMP22 using Method 1. The study used a capture probe/antibody system, with donor probes/antibodies labeled with the bioluminescent and acceptor probes/antibodies with fluorescence tags on a nanoparticle array. AuNRs functionalized with anti-NanoLuc antibodies were introduced at 1.0 OD. Various PEG spacer lengths were tested, similar to a previous study with AuSPs. Results showed that a PEG4-SH spacer (˜200 g/mol) provided maximum BRET enhancement for both mRNA and protein amplification (FIG. 6K, blue and red lines). This optimal spacer length was shorter than that found for AuSPs in protein applications, possibly due to differences in nanoparticle shape, slight variations in wavelength, and the additional−6-8 nm gap created by the anti-NanoLuc antibody binding to the bioluminescent tag (NanoLuc protein) (Table 1).

The AuNRs-S-PEG4-NH-antiNanoluc antibody complex serves as a universal signal enhancer (USE) for the MIP technology, applicable to all RNA and Protein applications. Its effectiveness stems from direct binding to the donor-bioluminescent molecule, actively participating in BRET/bioluminescent enhancement. This direct interaction allows for efficient BRET detection even at low analyte concentrations, as a single nanoparticle can provide significant enhancement. To determine the optimal concentration of USE various optical densities, ODs of AuNRs were tested at the previously determined distance. The study revealed that maximum enhancement was achieved at 0.25 OD of AuNRs (FIG. 6L). FIG. 6L illustrates a graph showing optimum AuNRs concentration for universal signal enhancer solution. Solutions of AuNRs-S-PEG-NH-anti NanoLuc antibodies with varying optical densities (ODs) were evaluated to optimize signal enhancement of the MIP assay utilizing bioluminescent resonance energy transfer (BRET). The highest BRET enhancement was achieved with an AuNRs solution at an OD of 0.25.

Based on these findings, the MIP application now employs a universal signal enhancer solution consisting of 0.25 OD AuNRs-S-PEG-NH-anti NanoLuc antibody. This standardized approach allows for consistent and efficient BRET enhancement across different MIP applications. Based on this optimization, full calibration plots for the ANXA10 and NUMA protein with plasmonic coupling of two nanoparticles were completed and it was found that 100 times more sensitivity compared to using of single nanoparticles (AuSPs) on the nanoparticle array. (FIG. 6I and FIG. 6J, red line).

Additionally, Finite-Difference Time-Domain (FDTD) simulations (FIG. 6M) were conducted to comprehend the plasmon enhancement resulting from the coupling of two nanoparticles. FIG. 6M illustrates an image of FDTD simulation of electromagnetic field enhancement upon AuSP and AuNR coupling.

These results demonstrate that the optimized universal signal enhancer significantly improves the sensitivity and performance of the MIP technology, enabling more efficient detection of low-abundance molecules in multiomic applications.

### Example 2

### Application of NanoBRET Using NanoLuc-Ligand 618 BRET Pair (Method 1)

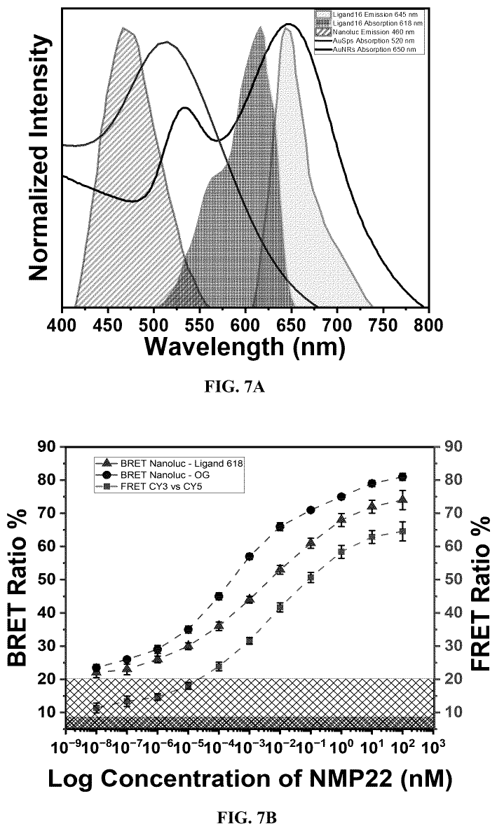

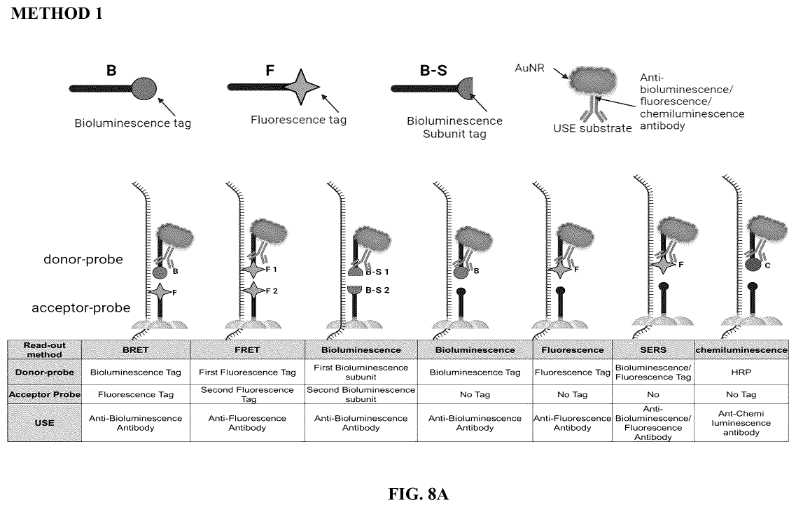

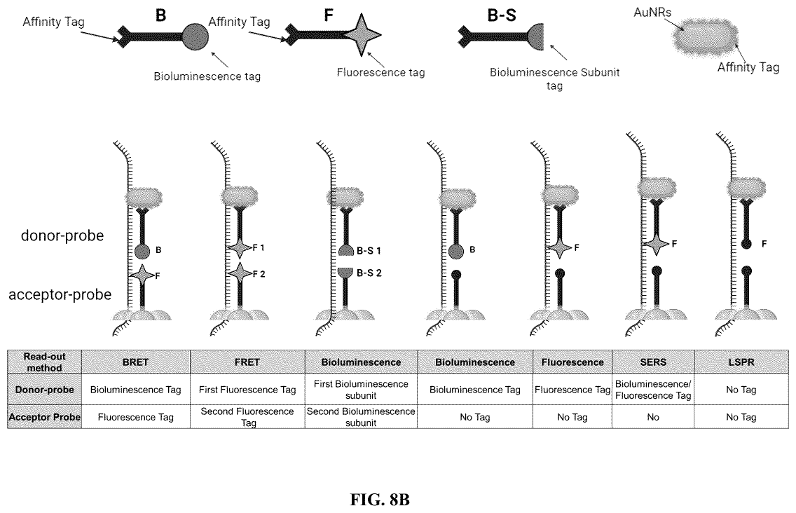

This example describes the application of NanoBRET using the NanoLuc-ligand 618 BRET pair (Method 1) in the Multi-Omic Integration Platform (MIP) technology. After refining the assay performance using the NanoLuc-OG BRET pair, the same parameters were applied to a NanoBRET application with NanoLuc-ligand 618, which demonstrates notable separation between the peaks of bioluminescent and fluorescence. In this configuration, a maximum LSPR peak of 520 nm (scattering profile ranging from 400 to 700 nm) was employed to excite bioluminescent emissions and a 650 nm LSPR peak from AuNRs for the absorption peak of the 618 ligands, ensuring effective overlap for BRET enhancement with absorption spectrum of Ligand 618, at 618 nm and emission spectrum at 645 nm (dense and wavy pattern respectively) (FIG. 7A). Calibration plots were generated for NMP22 protein and it was noted that the limits of detection (LODs) were relatively similar for both applications (7.85 aM). Although an improved LOD for the 618-ligand application was anticipated due to the greater overlap between the absorption peak of the 618 ligand and the LSPR peak of the AuNRs, this maximum overlap did not significantly enhance overall sensitivity. A decrease in standard deviation was also noted, which is attributed to the considerable separation between the bioluminescent and fluorescence peaks (FIG. 7B). FIGS. 7A and 7B illustrate graphs showing comparisons of the performances of MIP assays using different BRET and FRET pairs. FIG. 7A shows the LSPR peaks of AuSPs (520 nm) and AuNRs (650 nm) alongside the emission spectrum of Nanoluc (460 nm, blue region) and the absorption (590 nm, orange region) and absorption spectrum of Ligand 618 at 618 nm and the emission spectrum at 645 nm (dense and wavy pattern, respectively). FIG. 7A highlights the maximum overlap LSPR peaks of nanoparticles with the excitation and emission spectrum of donor and acceptor tags. FIG. 7B presents a calibration plot for NMP22 (ranging from 100 Nm to 100 aM) on the MIP assay, using the Nanoluc-Ligand 618 BRET pair (red triangle), the Nanoluc-OG BRET pair (blue circles), and the CY3-CY5 FRET (magenta squares) pair. However, the system must be tested with ssDNA once the manufacturing challenges related to the synthesis of the acceptor antibody-HaloTag 618 ligand complex are resolved.

These results demonstrate the application of the NanoBRET system using NanoLuc-ligand 618 in the MIP technology, showing comparable sensitivity to the NanoLuc-OG system but with potentially improved precision due to greater peak separation. This advancement contributes to the versatility and robustness of the MIP platform for detecting low-abundance molecules in multiomic applications.

### Example 3

Implementation of FRET Assay Using CY3-CY5 Pair with Optimized Parameters (Method

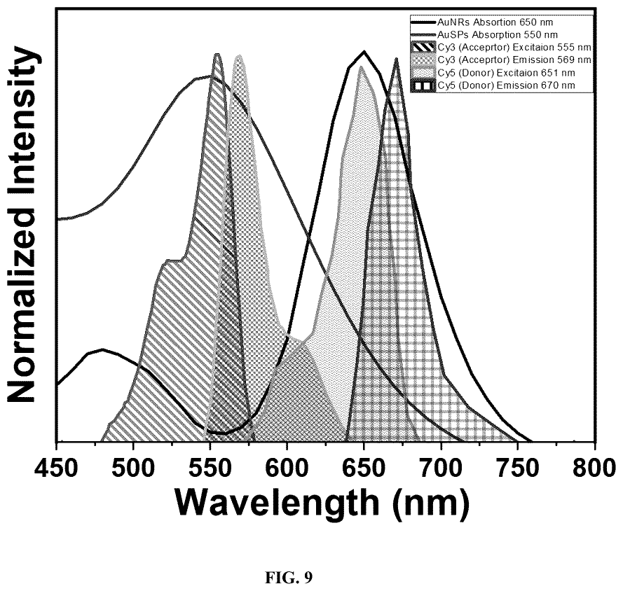

The assay was conducted using a FRET pair, with CY3 serving as the donor probe and CY5 as the acceptor probe, alongside AuNRs-S-PEG-NH-anti CY3 antibody while maintaining all previously optimized parameters. A 550 nm LSPR peak from the AuSP was used for the CY3 acceptor tag the excitation spectrum 555 nm-(medium cross pattern) and CY3 emission spectra 569 nm (cheque pattern). CY5 and excitation spectrum at 651 nm-(wavy pattern) and emission spectra 670 nm (cheque pattern) of CY5, also indicating maximum overlap (FIG. 9). FIG. 9 illustrates a graph showing the LSPR peaks of AuNPs (550 nm) red line and AuNRs (650 nm) black line. CY3 acceptor tag the excitation spectrum 555 nm-(medium cross pattern) and CY3 emission spectra 569 nm (cheque pattern). CY5 and excitation spectrum at 651 nm-(wavy pattern) and emission spectra 670 nm (cheque pattern) of CY5, also indicating maximum overlap. This configuration resulted in a limit of detection (LOD) of 23.891 Am for NMP22 applications (FIG. 7B, purple line).

NOTE: The optimization of LSPR-enhanced fluorescence emphasizes the broad scattering profile of nanoparticles rather than the precise overlap between LSPR and fluorophore absorption, particularly when the ideal distance between the nanoparticle and fluorophore is achieved. This method takes advantage of near-field effects, facilitates broadband enhancement, and accommodates different nanoparticle shapes. A broader scattering profile provides practical benefits, supporting various enhancement mechanisms and offering resilience against environmental changes. This approach is consistent with many studies that show significant fluorescence amplification can occur without perfect spectral matching, underscoring the importance of optimizing spatial parameters and utilizing the entire spectral range of plasmonic scattering for effective enhancement.

### Example 4

FRET Enhancement Analysis with Varying Nanoparticle Configurations Using Method 2

This example describes the implementation of a FRET assay using the CY3-CY5 pair with optimized parameters (Method 2) in the Multi-Omic Integration Platform (MIP) technology.

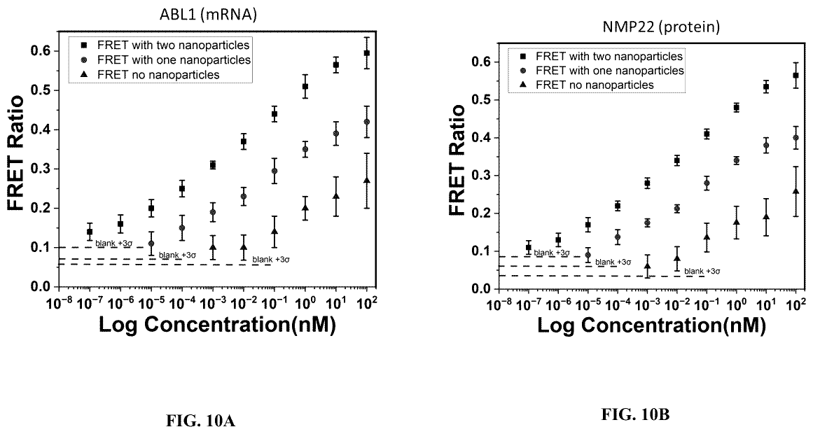

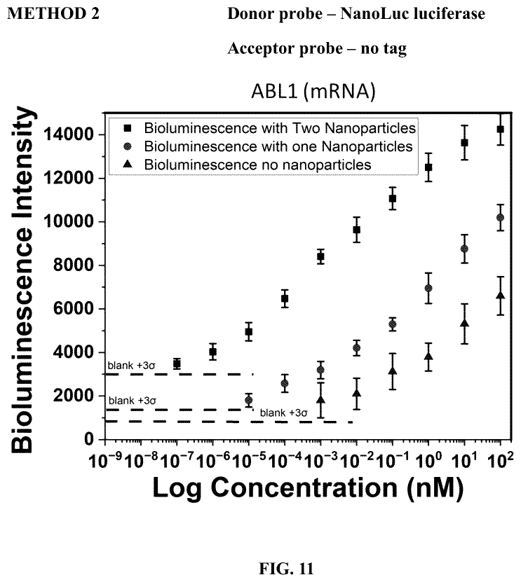

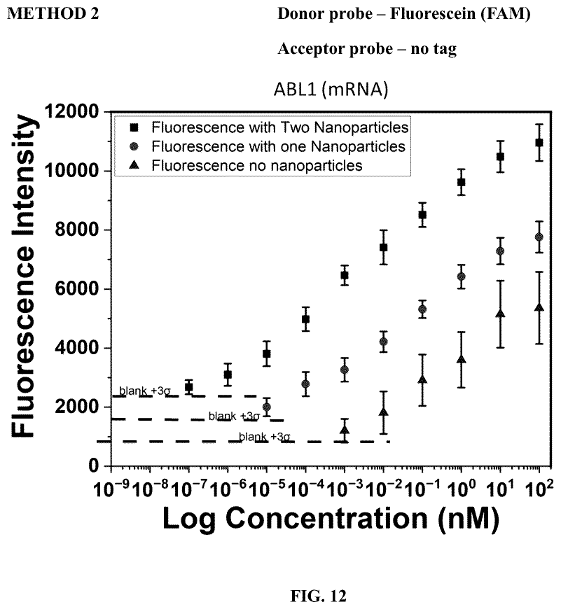

FRET enhancement was investigated using the method 2 where the second nanoparticle (AuNRs) attaches to the affinity tag of the donor probe or capture antibody. The same distance was maintained as AuNRs directly bind to the Acceptor tag. This study was carried out with no nanoparticles, with one nanoparticle, and with two nanoparticles. The findings indicate that in both protein and nucleic acid applications, adding one nanoparticle results in a logarithmic two-fold increase in sensitivity, while incorporating two nanoparticles further boosts sensitivity by an additional two logs, thereby enhancing the assay's overall sensitivity. FIG. 10 illustrates graphs showing calibration curves developed for MIP assay using Forster Resonance Energy Transfer (FRET) as a readout signal under three conditions for ABL1 (mRNA) and NMP22 (protein): Method 2 without nanoparticles for enhancement (blue triangle plot), with one nanoparticle for enhancement (red circle plot), and with two nanoparticles for enhancement (black square plot). FIG. 10A shows the FRET ratio using a FAM-tagged donor probe and a Cy5-tagged acceptor probe, corresponding to different concentrations of ABL1 (mRNA) analyte in accordance with the present disclosure. The limit of detection identified in the plot demonstrates an improvement of approximately 2 logs and 4 logs, respectively, compared to the FRET complex with one nanoparticle and no nanoparticles. FIG. 10B shows FRET ratio using a FAM-tagged donor antibody and a Cy5-tagged acceptor antibody, corresponding to different concentrations of NMP22 protein analyte in accordance with the present disclosure. The limit of detection identified in the plot demonstrates an improvement of approximately 2 logs and 4 logs, respectively, compared to the FRET complex with one nanoparticle and no nanoparticles.

These results demonstrate the successful implementation of a FRET-based assay in the MIP technology using the CY3-CY5 pair. The achieved limit of detection and the notes on LSPR-enhanced fluorescence highlight the versatility and robustness of the MIP platform for detecting low-abundance molecules in multiomic applications, emphasizing the importance of considering both spectral and spatial factors in assay optimization.

### Example 5

Bioluminescent Enhancement Study with Different Nanoparticle Arrangements Using Method 2

This example describes a study on bioluminescent enhancement using different nanoparticle arrangements in the Multi-Omic Integration Platform (MIP) technology, employing Method 2.

The study compared three conditions: without nanoparticles, with one nanoparticle, and with two nanoparticles. It used the second approach where the second nanoparticle binds to the affinity tag of the donor probe or capture antibody. The donor probe was equipped with a NanoLuc bioluminescent tag, while the acceptor probe had no tag, allowing for a bioluminescent assay upon hybridization.

For mRNA ABL1 nucleic acids applications, the findings show that the addition of one nanoparticle leads to a logarithmic two-fold increase in sensitivity. Incorporating a second nanoparticle further boosts the sensitivity by another two logs, thus significantly enhancing the assay's overall sensitivity.

FIG. 11 illustrates these findings with a graph showing calibration curves for the MIP assay using Bioluminescent as a readout signal. The graph plots three conditions: Method 2 without nanoparticles (blue triangle plot), with one nanoparticle for enhancement (red circle plot), and with two nanoparticles for enhancement (black square plot). The x-axis represents concentrations of ABL1 (mRNA) analyte, while the y-axis shows bioluminescent intensity. The limit of detection demonstrated in the plot shows an improvement of approximately 2 logs with one nanoparticle and 4 logs with two nanoparticles, compared to the bioluminescent with no nanoparticles.

These results demonstrate the significant enhancement in sensitivity achieved by incorporating nanoparticles in the MIP technology using Method 2. The use of two nanoparticles provides a substantial improvement in the limit of detection for mRNA ABL1, highlighting the potential of this approach for detecting extremely low-abundance molecules in multiomic applications. This advancement in sensitivity could have important implications for early disease detection and precision medicine.

### Example 6

Fluorescence Enhancement Investigation with Various Nanoparticle Setups Using Method 2

This example describes a study on fluorescence enhancement using different nanoparticle arrangements in the Multi-Omic Integration Platform (MIP) technology, employing Method 2.

The study explored fluorescence enhancement in three scenarios: without nanoparticles, with one nanoparticle, and with two nanoparticles. In this investigation, the donor probe was tagged with FAM (Fluorescein), and the experiments utilized the second approach, where the second nanoparticle (AuNRs) attached to the affinity tag of the donor probe.

For mRNA ABL1 nucleic acids applications, the results showed that the inclusion of one nanoparticle led to a logarithmic two-fold increase in sensitivity. Adding a second nanoparticle further enhanced the sensitivity by an additional two logs, significantly improving the assay's overall sensitivity.

FIG. 12 illustrates these findings with a graph showing calibration curves for the MIP assay using fluorescence as a readout signal. The graph plots three conditions: Method 2 without nanoparticles for enhancement (blue triangle plot), with one nanoparticle for enhancement (red circle plot), and with two nanoparticles for enhancement (black square plot). The x-axis represents concentrations of ABL1 (mRNA) analyte, while the y-axis shows fluorescence intensity. The fluorescence intensity corresponds to different concentrations of ABL1 (mRNA) analyte, using a Fluorescein (FAM) tagged donor probe and an untagged acceptor probe.

The limit of detection demonstrated in the plot shows an improvement of approximately 2 logs with one nanoparticle and 4 logs with two nanoparticles, compared to the fluorescence with no nanoparticles. This significant enhancement in sensitivity highlights the potential of this approach for detecting extremely low-abundance molecules in multiomic applications.

These results demonstrate the substantial improvement in fluorescence-based detection achieved by incorporating nanoparticles in the MIP technology using Method 2. The use of two nanoparticles provides a marked enhancement in the limit of detection for mRNA ABL1, which could have important implications for early disease detection and precision medicine.

### Example 7

LSPR-Based Detection without Label Tags Using Method 2

This example describes a study on Localized Surface Plasmon Resonance (LSPR)-based detection without label tags in the Multi-Omic Integration Platform (MIP) technology, employing Method 2.

The experiments were conducted without tagging either the donor or acceptor probes. Instead, the study monitored the LSPR peak via the absorption peak of the nanoparticles. This investigation utilized the second approach, where the second nanoparticle (AuNRs) binds to the affinity tag of the donor probe following the final hybridization with the acceptor probe.

The results showed that the AuNPs' peak at 520 nm remained constant throughout the experiment. However, the intensity of the second nanoparticle (AuNRs) gradually increased around ˜650 nm as the analyte concentrations increased. This gradual increase was observed across a concentration range from 10 aM to 100 nM.

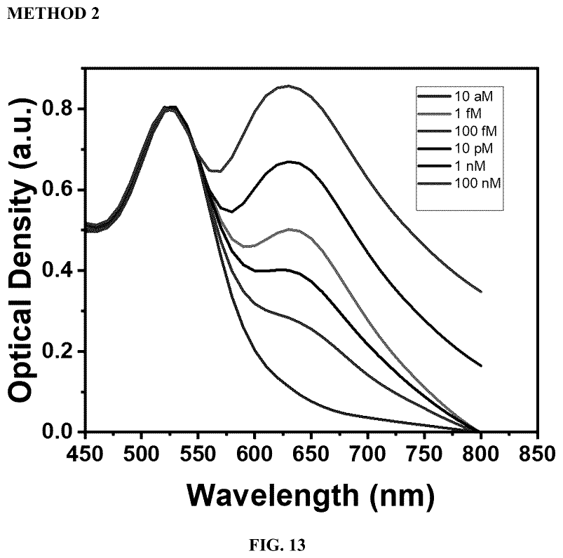

FIG. 13 illustrates these findings with a graph showing localized surface plasmon resonance as a readout signal for the MIP assay without any label. The graph demonstrates how the LSPR signal of the nanoparticle array surface changed after hybridization with the target analyte across the concentration range of 10 aM to 100 nM. The LSPR peak of AuSP on the nanoparticle array surface remained constant at 520 nm with the same intensity. The AuNRs peak concentration increased upon the addition of AuNRs, transitioning through purple, black, green, blue, and red lines. The lowest AuNRs peak appeared at 10 aM, and the highest peak appeared at 100 nM.

These results demonstrate the potential of using LSPR-based detection without label tags in the MIP technology. This approach allows for the detection of analytes across a wide concentration range (10 aM to 100 nM) by monitoring the changes in the LSPR signal of the nanoparticles. The ability to detect analytes without the need for labeling could simplify assay procedures and potentially reduce costs, while still maintaining high sensitivity. This method could have significant applications in various fields, including diagnostics and environmental monitoring, where label-free detection of low-abundance molecules is desirable.

### Example 8

### Calibration Plots in Urine Equivalent Using Method 1

This example describes the development of calibration plots in urine equivalent using Method 1 for the Multi-Omic Integration Platform (MIP) technology. Standard curves were developed for a range of analytes: 5 mRNA, 1 lncRNA, 4 miRNAs, and 2 proteins. These curves were generated using standard analytes for the first approach, where the signal analyte enhancer solution directly binds to the Bioluminescent tag. The sequences of the analytes are provided in Table 2.

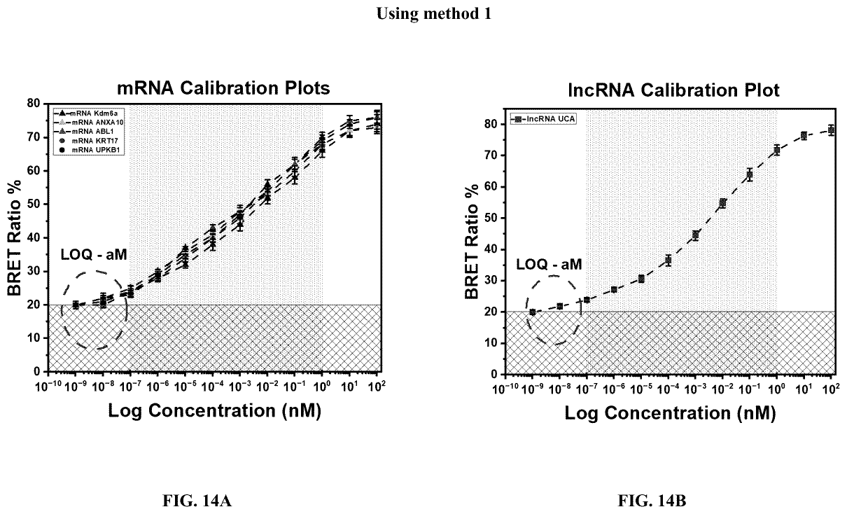

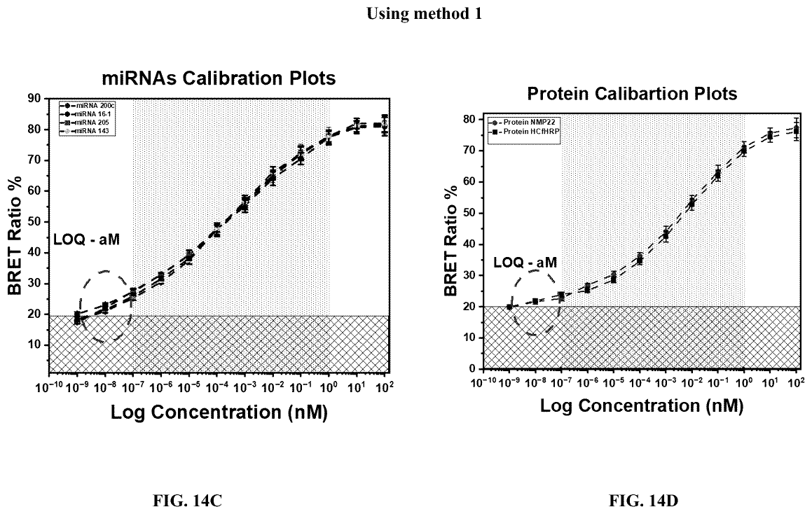

FIGS. 14A-14D illustrate the calibration plots for the different types of analytes: FIG. 14A shows the calibration plots for mRNA. FIG. 14B presents the calibration plot for lncRNA. FIG. 14C displays the calibration plots for miRNAs. FIG. 14D illustrates the calibration plots for Proteins. These figures present the measurement results for each of the 12 analytes in spiked human urine samples using Method 1. Control signal values, shown as BRET ratios, were recorded at 100 nM and 1 aM (10-9 nM). The assay demonstrated high linearity across eight orders of magnitude in concentration, ranging from 1 nM to 10 aM. In the graphs, the darkened area highlights the most linear region of the calibration curves, while the crosshatched area indicates the blank (mean+3 standard deviations). Data points are plotted on a logarithmic scale, with each point representing an average of six replicates across six different sensors. The standard error for each point is also included.

TABLE 2
MIP assay performance metrics across different readout signals and analytes.
METHOD 1

| Assay Type/ | Marker | Linear Regression | | LOD | LOD |
|---|---|---|---|---|---|
| Specifications | Type | Equation | ULOQ-LLOQ | (aM) | comparison |
| Nanoluc/ | Protein NMP22 | y = 2.9828ln(x) + 75.65 | 100 nM-100 aM | 7.8595 | |
| ligand 618 | | | | | |
| Nanoluc/OG | Protein NMP22 | y = 2.2656ln(x) + 48.878 | 100 nM-100 aM | 25.918 | |
| CY3/CY5 | Protein NMP22 | y = 2.6965ln(x) + 55.317 | 100 nM-100 aM | 23.891 | |
| Nanoluc/OG | mRNA ANXA 10 | y = 2.5821ln(x) + 65.291 | 100 nM-10 aM | 24.088 | 95.17-fold higher |
| with 2 NPs | | | | | than 1NPs |
| Nanoluc/OG | mRNA ANXA 11 | y = 2.4393ln(x) + 51.678 | 100 nM-1 fM | 2292.4 | 98.74-fold higher |
| with 1 NPs | | | | | than no Nps |
| Nanoluc/OG | mRNA ANXA 12 | y = 2.7981ln(x) + 31.276 | 100 nM-1 pM | 226350 | |
| With No NPs | | | | | |
| Nanoluc/OG | Protein NMP22 | y = 2.6255ln(x) + 65.864 | 100 nM-100 aM | 25.974 | |
| with 2 NPs | | | | | |
| Nanoluc/OG | Protein NMP22 | y = 2.2656ln(x) + 48.878 | 100 nM-1 fM | 2916.5 | 112.29-fold higher |
| with 1 NPs | | | | | than 1Nps |
| Nanoluc/OG | Protein NMP22 | y = 2.2483ln(x) + 28.178 | 100 nM-1 pM | 307840 | 105.55-fold higher |
| With No NPs | | | | | than no Nps |
| Nanoluc/OG | mRNA ABL1 | y = 2.6966ln(x) + 66.718 | 100 nM-10 aM | 27.399 | |
| Nanoluc/OG | mRNA ANXA10 | y = 2.586ln(x) + 64.86 | 100 nM-10 aM | 10.159 | |
| Nanoluc/OG | mRNA KDM6A | y = 2.515ln(x) + 63.645 | 100 nM-10 aM | 28.93 | |
| Nanoluc/OG | mRNA UPKB1 | y = 2.5229ln(x) + 65.518 | 100 nM-10 aM | 14.584 | |
| Nanoluc/OG | mRNA KRT17 | y = 2.7065ln(x) + 68.098 | 100 nM-10 aM | 15.204 | |
| Nanoluc/OG | lncRNA UCA | y = 2.8183ln(x) + 68.294 | 100 nM-10 aM | 36.068 | |
| Nanoluc/OG | Protein NMP22 | y = 2.8071ln(x) + 67.664 | 100 nM-10 aM | 42.039 | |
| Nanoluc/OG | Protein HCFHrp | y = 2.7795ln(x) + 65.745 | 100 nM-10 aM | 71.215 | |
| Nanoluc/OG | miRNA 200c | y = 2.941ln(x) + 74.664 | 100 nM-10 aM | 8.4843 | |
| Nanoluc/OG | miRNA 16-1 | y = 2.8226ln(x) + 73.944 | 100 nM-10 aM | 5.0156 | |
| Nanoluc/OG | miRNA 205 | y = 2.9092ln(x) + 75.119 | 100 nM-10 aM | 5.9037 | |
| Nanoluc/OG | miRNA 143 | y = 2.9408ln(x) + 74.673 | 101 nM-10 aM | 8.4079 | |

These results demonstrate the broad applicability and high sensitivity of the MTP technology using Method 1 for detecting various types of analytes in urine samples. The wide linear range (1 nM to 10 aM) and the ability to detect multiple types of biomolecules (mRNA, lncRNA, miRNAs, and proteins) highlight the versatility of this approach for multiomics applications. The use of urine equivalent samples also suggests the potential of this method for non-invasive diagnostics and biomarker detection.

### Example 9

### Specificity and Selectivity Test Using Method 1

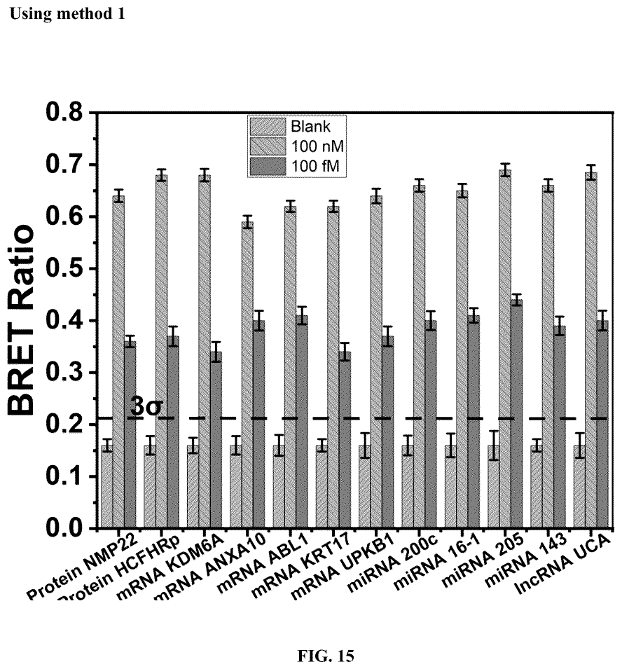

This example describes the specificity and selectivity test for the Multi-Omic Integration Platform (MTP) technology using Method 1. The prepared sensors were tested for specificity against four selected markers representing all four categories of omics: protein, mRNA, miRNA, and lncRNA. The experiment was conducted in two stages: First, a high-concentration mixture (1 μM) of all analyte types excluding the target was prepared and tested. This resulted in a BRET ratio below the blank's limit, indicated by purple color columns in FIG. 15. Subsequently, tests were conducted with the target analyte included. These tests demonstrated the sensor's accuracy, yielding an expected BRET ratio with less than 2.3% coefficient of variation. In FIG. 15, these results are represented by red columns for 100 nM concentration and blue columns for 100 fM concentration.

FIG. 15 illustrates these findings with a bar graph showing MIP assay specificity. The graph displays the results for each multiomics category: protein, mRNA, miRNA, and lncRNA. The purple columns represent the BRET ratio for the high-concentration mixture without the target analyte. The red and blue columns show the BRET ratios when the target analyte is included at 100 nM and 100 fM concentrations, respectively. These findings confirm the high specificity of the protein, mRNA, miRNA, and lncRNA assays developed using the MIP technology. Significant BRET signals were only detected in the presence of the target analyte, ensuring the absence of false positives.

These results demonstrate the excellent specificity and selectivity of the MIP assay using Method 1. The ability to accurately detect target analytes across different omics categories, even in the presence of high concentrations of non-target molecules, highlights the robustness of this approach. This high specificity is crucial for reliable multiomics analysis, particularly in complex biological samples where multiple biomolecules are present. The low coefficient of variation (less than 2.3%) also indicates good reproducibility of the assay, further supporting its potential for various diagnostic and research applications.

### Example 10

### Reproducibility of the MIP Assay Using Method 1

This example describes the reproducibility test for the Multi-Omic Integration Platform (MIP) technology using Method 1. The reproducibility of the MIP technology was measured using selected multiomic biomarkers representing each category: protein (NMP22), miRNA (16-1), mRNA (ANXA10-1), and lncRNA (UCA). These biomarkers were tested at both high and low concentrations. BRET ratios were recorded after hybridization/conjugation on three independently prepared assay batches. Each batch was tested at four different concentrations: 100 nM, 100 pM, 100 fM, and 100 aM. For each concentration, six technical replicates were performed. The results showed average coefficients of variation (CV) of less than 2.88% for 100 nM, less than 2.92% for 100 pM, less than 1.95% for 100 fM, and less than 2.13% for 100 aM. These results demonstrate consistent assay performance with an overall CV of less than 3%. Notably, the CVs for lower concentrations were significantly lower than those for higher concentrations, highlighting enhanced stability and reliability at minimal analyte levels.

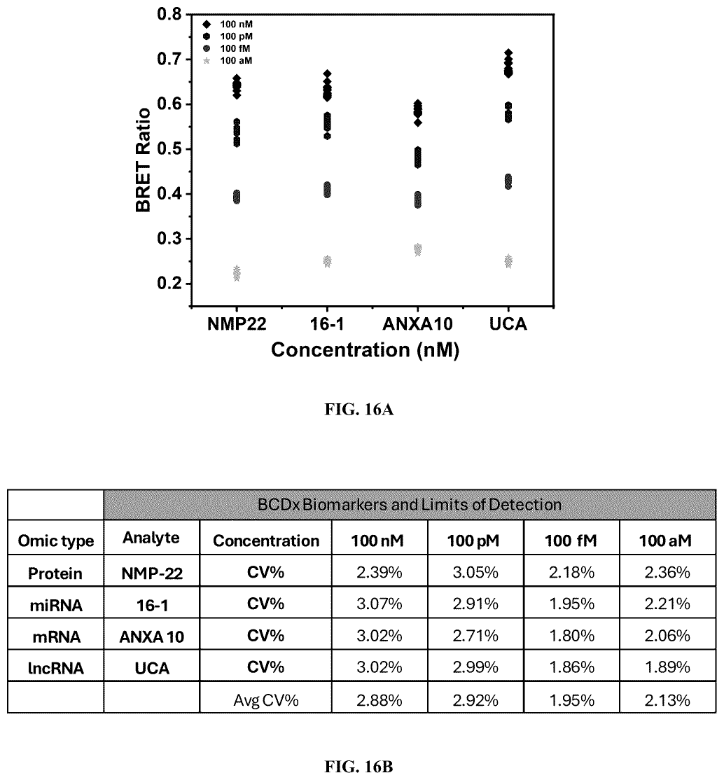

FIG. 16A illustrates a graph showing the reproducibility tested for each omic category at varying concentrations. It displays the BRET ratios recorded for the biomarkers after hybridization/conjugation on the three independently prepared assay batches. FIG. 16B presents a table showing the average coefficients of variation (CVs) for each concentration tested.

These results demonstrate the high reproducibility of the MIP assay using Method 1 across different types of biomarkers and a wide range of concentrations. The consistently low CVs, especially at lower concentrations, indicate the robustness and reliability of the assay for detecting low-abundance molecules. This high reproducibility is crucial for the application of the MIP technology in various fields, including clinical diagnostics and research, where consistent and reliable results are essential.

### Example 11

### Laboratory and Operator Repeatability and Reproducibility Using Method 1

This example describes the laboratory and operator repeatability and reproducibility test for the Multi-Omic Integration Platform (MIP) technology using Method 1. The evaluation was conducted across two different laboratories: the Indiana Bioscience Research Institute and the Indiana Center of Biomedical Innovation, both located in Indianapolis, Indiana, USA. Two operators at each site performed tests over three non-consecutive days using three different batches of MIP assays. The assays were specifically functionalized for four biomarkers: protein (NMP22), miRNA (16-1), mRNA (ANXA10-1), and lncRNA (UCA). Each operator managed nine assays per biomarker, prepared from separate nanoparticle batches. They used a stock solution of high (100 pM) and low (100 aM) concentrations in human urine equivalent to perform tests on a GloMax Discover plate reader, ensuring consistency across setups. The recorded BRET ratios were compared to the intensity of the 100 pM stock solution. The resulting coefficient of variation was less than 1.6%, demonstrating high reproducibility and repeatability of the MIP technology across different settings and operators.

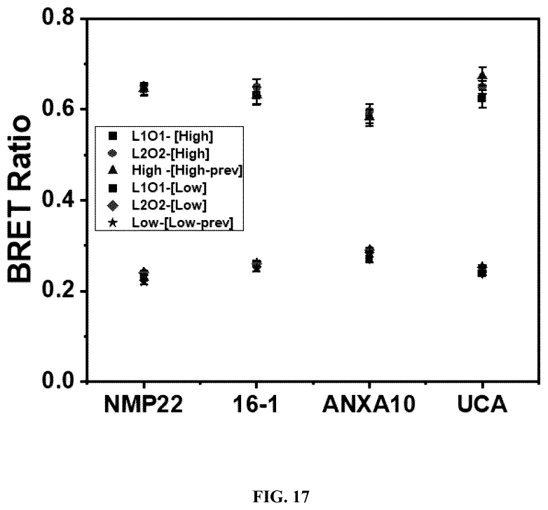

FIG. 17 illustrates a graph showing the repeatability and reproducibility of the MIP assay. It displays the results from the tests conducted by different operators across the two laboratories, highlighting the consistency of the BRET ratios obtained for each biomarker at both high and low concentrations.

These results demonstrate the robustness of the MIP technology using Method 1. The low coefficient of variation across different laboratories, operators, and days indicates that the assay produces consistent results regardless of the testing environment or the person conducting the test. This high level of repeatability and reproducibility is crucial for the potential clinical application of the MIP technology, as it ensures reliable results across different settings and operators. Such consistency is essential for diagnostic tools, particularly when detecting low-abundance biomarkers that could be indicative of early-stage diseases or conditions.

### Example 12

Comparison of MIP Assay Sensitivity and Linearity with qPCR and ELISA Using Method 1

This example describes a comparison of the Multi-Omic Integration Platform (MIP) assay sensitivity and linearity with qPCR and ELISA using Method 1.

The study investigated the sensitivity and linearity of multiomic measurements using MIP assay technology against conventional RT-PCR for nucleic acids and ELISA for proteins. For nucleic acid detection, sensitivity was evaluated by analyzing mRNA ABL1 with a G-block sequence in spiked urine samples. Separate standard curves were developed for each method.

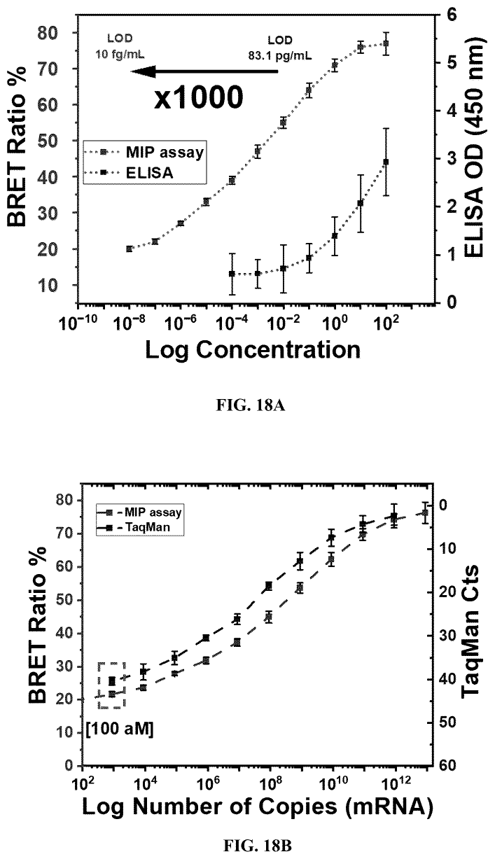

FIG. 18A illustrates the comparison between MIP and RT-PCR for nucleic acid detection. Both methods achieved similar sensitivity within the attomolar range (approximately 8,000 copies). At lower concentrations, both MIP and RT-PCR showed an increase in TaqMan counts, suggesting assay variations.

For protein detection, the MIP technology was compared to ELISA. FIG. 18B demonstrates that MIP technology not only showed enhanced signals correlating with increasing analyte concentrations but also exhibited a broader linear range from 100 nM to 10\hat{ }-8 nM.

The detection limit for the NMP22 ELISA was 83.1 μg/mL. However, this was significantly improved to 10 fg/mL by MIP-enhanced plasmonic ELISA, enhancing sensitivity by a factor of 1000.

These results demonstrate that the MIP assay technology provides comparable or superior performance to conventional methods for both nucleic acid and protein detection. For nucleic acids, MIP showed similar sensitivity to RT-PCR, while for proteins, it significantly outperformed traditional ELISA in terms of both sensitivity and linear range. The thousand-fold improvement in protein detection sensitivity highlights the potential of MIP technology for detecting low-abundance biomarkers in complex biological samples. This enhanced performance could have significant implications for early disease detection and monitoring, where the ability to detect minute quantities of biomarkers is crucial.

### Example 13

### Evaluation of the MIP Technology for RNA and Protein Detection in BC and Healthy Samples Using Method 1

This example describes the evaluation of the Multi-Omic Integration Platform (MIP) technology for RNA and protein detection in bladder cancer (BC) and healthy samples using Method 1. To demonstrate the ability of detecting multiomics markers in human urine, the assay was deployed for 4 different omic markers: miRNA 205, mRNA ANXA10, lncRNA UCA, and Protein NMP22 on 12 bladder cancer urine samples and 12 healthy samples (IRB 1R44CA291290-01).

MIP Workflow for miRNAs, mRNAs and lncRNAs in Direct Urine

Urine aliquots (1 mL each) were processed by adding 460 μL of lysis buffer and 160 μL of proteinase K. The samples were vortexed for 60 seconds and incubated at 37° C. for 15 minutes. Targeted capture probes (1 μg/mL) were directly added to the lysed urine sample, and the mixture was incubated at 37° C. with gentle shaking for 2 hours. Streptavidin-coated magnetic beads (0.25 mg/mL) were prepared according to the manufacturer's protocol and resuspended in 2× binding and washing buffer (B&W buffer) to a final concentration of 0.25 mg/mL. These beads were added to the urine samples to capture the probe-target complexes at 37° C. within 30 minutes. After incubation, the beads were separated and subjected to 4-5 washes with washing buffer.

The separated beads were resuspended in 100 μL of binding buffer and transferred to a Corning 96-well plate. A solution containing targeted pre-functionalized donor probes (10 μg/mL, 100 μL) was added to each well. The mixture was incubated for one hour at a temperature approximately 5 degrees below the average melting temperature of all the probes.

The magnetic beads were separated and washed three times with W&B buffer. The beads were then resuspended in 100 μL of W&B buffer. The solution was exposed to nonharmful UV light at 365 nm for 2 minutes using a SynLED UV instrument (Catalog No: C7444081). After UV exposure, the solution was transferred to the targeted wells pre-functionalized with acceptor probes specific to the target analyte on the nanoparticle array surface. This mixture was incubated for two hours at 37° C.

Following aspiration of excess solution, 100 μL of single analyte enhancer solution in binding buffer was added, and the reaction continued for 1 hr. Excess material was then aspirated. Finally, 50 μL of 1:10 NanoLuc substrate with NanoLuc buffer solution was added to each well, and bioluminescent and fluorescence results were collected using a BioTek microplate reader.

The blank-corrected obtained intensity was converted to concentration using pre-developed calibration plots.

### MIP Workflow for Proteins

Urine aliquots (1 mL each) were subjected to a freeze-thaw cycle followed by vortexing for 60 seconds. Targeted capture probe (50 μg/mL) was directly added to the samples, which were then incubated at 37° C. for 2 hours. Streptavidin-coated magnetic beads (0.05 mg/mL) were prepared according to the manufacturer's protocol, washed, and resuspended in PBS (pH 7.4) to a final concentration of 0.05 mg/mL. These beads were added to the urine samples to capture the target protein complexes at 37° C. within 30 minutes. After incubation, the coated beads were washed 4-5 times with PBS-T solution containing 0.1% BSA.

The separated beads were resuspended in 100 μL of binding buffer and transferred to a Greiner Bio-one non-binding 96-well microplate. A targeted donor probe mixture (10 μg/mL, 50 μL) in PBS (pH 7.4) was added, and the reaction continued at 37° C. for one hour.

The magnetic beads were separated, washed twice with washing buffer, and resuspended in 100 μL of PBS (pH 7.4). The solution was exposed to nonharmful UV light at 365 nm for 2 minutes using a SynLED UV instrument (Catalog No: C7444081). After UV exposure, the solution was transferred to target wells pre-functionalized with acceptor antibodies specific to the target analyte on the nanoparticle array surface on white half-well plates with glass bottoms. This mixture was incubated for two hours.

Following aspiration of excess solution, 100 μL of single analyte enhancer solution in binding buffer was added, and the reaction continued for 1 hour. The bottom of the well plate was blocked with BRAND® sealing film for microplates. Excess material was then aspirated. Finally, 50 μL of 1:10 NanoLuc substrate with NanoLuc buffer solution was added to each well, and bioluminescent and fluorescence results were collected using a BioTek microplate reader.

The blank-corrected obtained intensity was converted to concentration using pre-developed calibration plots.

For each analyte, the assay was repeated in triplicate. In total, for the MIP assay, one patient sample was distributed across 36 wells, including controls and housekeeping genes for different applications.

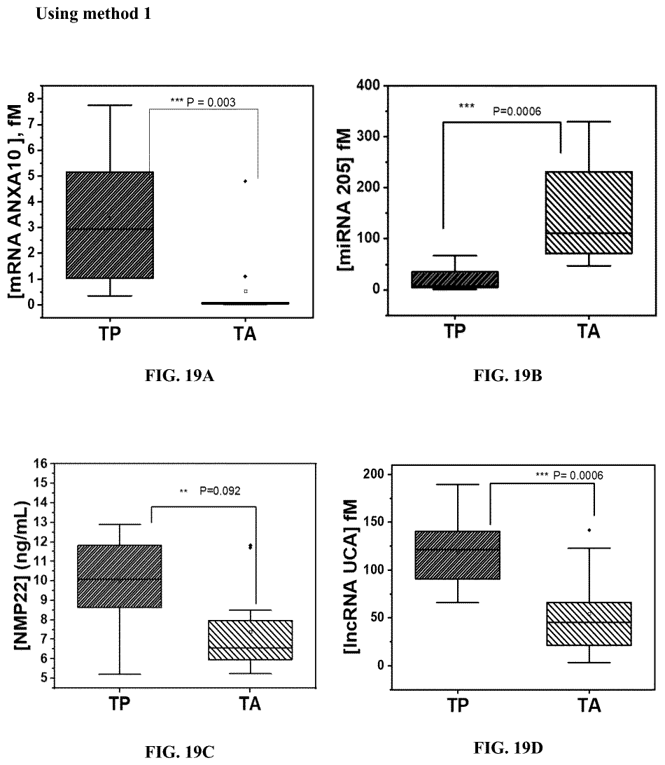

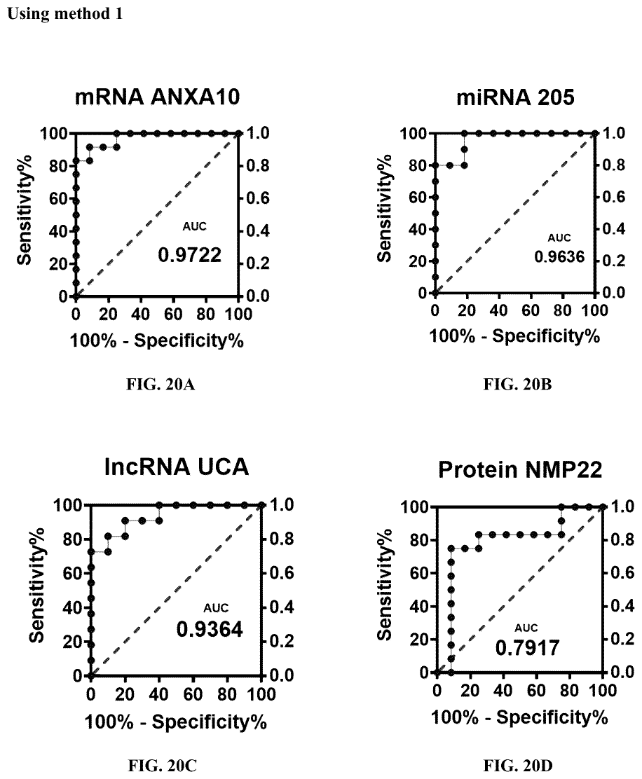

Statistical analyses were performed using GraphPad Prism 9.5.1 and Origin software. Differences with P<0.05 were considered statistically significant. Receiver operating characteristic (ROC) curves were utilized to assess sensitivity and specificity. The figures were created using GraphPad Prism and Origin software. All data points represent three or more biological or technical replicates, as indicated for each experiment (FIGS. 19A-D and FIGS. 20 A-D). FIGS. 19A-D illustrate box plots showing the expression levels (y-axis: concentration in fM) of the multiomics markers mRNA ANXA10 (FIG. 19A), miRNA 205 (FIG. 19B), lncRNA UCA (FIG. 19C) and Protein NMP22 (FIG. 19D) in urine from 12 patients with tumor present (TP) and 12 healthy individuals/tumor absent (TA). Concentrations were determined using calibration plots from (FIGS. 14 A-D). P-values, signifying the statistical significance of differences between the groups, are noted on each box plot, highlighting which markers significantly differentiate between the tumor presence and absence.

FIGS. 20A-D illustrate receiver operating characteristic (ROC) curves for each biomarker's mRNA ANXA10 (FIG. 20A), miRNA 205 (FIG. 20B), lncRNA UCA (FIG. 20C) and Protein NMP22 (FIG. 20D) predicting the presence of tumors in the patient and healthy cohort. The AUC values indicate the diagnostic accuracy of each biomarker.

These results demonstrate the capability of the MIP technology to detect multiple types of biomarkers (miRNA, mRNA, lncRNA, and protein) in urine samples from both bladder cancer patients and healthy individuals. The statistical analyses and ROC curves provide insights into the diagnostic potential of these biomarkers for distinguishing between tumor presence and absence. This multi-omics approach could potentially improve the accuracy and reliability of bladder cancer detection and monitoring.

### Example 14

### Universal Signal Enhancer (USE) for Sensitivity Increment of HRP-Chemiluminescence Assay

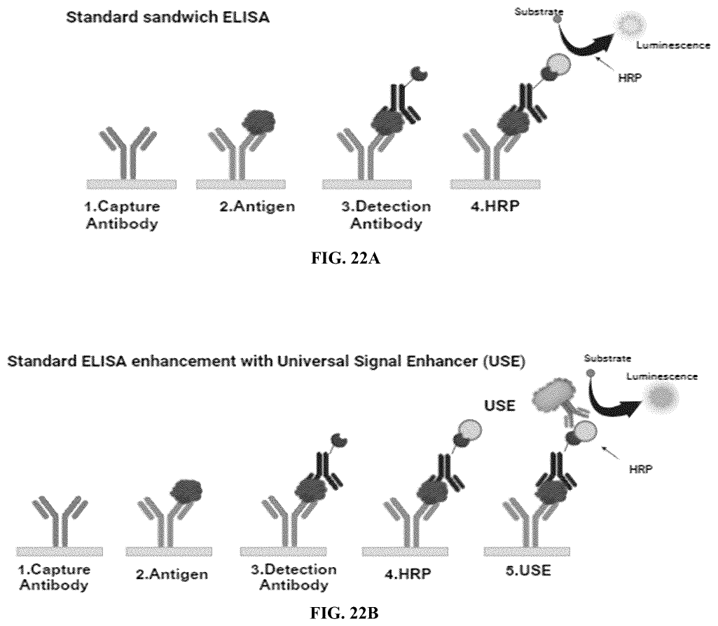

This example describes the use of a Universal Signal Enhancer (USE) to increase the sensitivity of HRP-Chemiluminescence Assay in ELISA. The study first outlines a standard sandwich ELISA procedure, as illustrated in FIG. 22. This involves coating a capture antibody onto a well plate, capturing antigens from the specimen, binding a biotin-tagged detection antibody, adding HRP-conjugated streptavidin, and finally introducing a chemiluminescent substrate to produce luminescence.

FIG. 22B illustrates a modified sandwich ELISA assay with an additional step involving the addition of a universal enhancer solution (USE) containing AuNRs (gold nanorods) conjugated with an anti-HRP antibody. This bound to the HRP enzyme, which had been attached to the detection antibody via a streptavidin-biotin interaction. Following this, a chemiluminescent substrate was added to complete the assay. The inclusion of the USE solution enhanced the sensitivity of the standard sandwich ELISA.

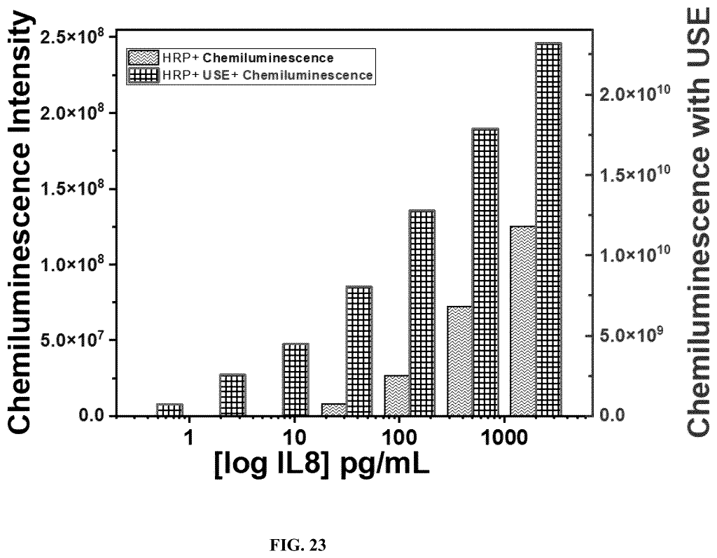

A comparative study was conducted for IL-8 protein marker detection using the standard sandwich ELISA and the USE-enhanced standard sandwich ELISA. FIG. 23 illustrates a bar graph comparing IL-8 protein marker detection using standard sandwich ELISA and sandwich ELISA with universal signal enhancer (USE). The left axis (blue wavy bars) shows the detection range without USE (2000 pg/mL to 31.25 pg/mL), while the right axis (red check bars) demonstrates the expanded sensitivity achieved with USE (2000 pg/mL to 0.031 pg/mL). Results highlight the significant improvement in signal-to-noise ratio and potential for increased lower limit of quantification (LLOQ) using the USE method. The left axis (blue wavy bars) in FIG. 23 shows the detection range without USE (2000 pg/mL to 31.25 pg/mL), while the right axis (red check bars) demonstrates the expanded sensitivity achieved with USE (2000 pg/mL to 0.031 pg/mL). Results highlight the significant improvement in signal-to-noise ratio and potential for increased lower limit of quantification (LLOQ) using the USE method.

These findings confirm the effectiveness of incorporating the Universal Signal Enhancer into standard chemiluminescence-based ELISA, substantially improving sensitivity with a signal-to-noise (S/N) ratio at the lower limit of quantification (LLOQ) that is ten times above the background level. This enhanced approach is not only more sensitive but also compatible with highly automated systems such as the Cobas® modular analyzer from Roche, UniCel-DxI-600 from Beckman Coulter, and other standardized automated immunoassay platforms. The integration of USE in Vitros®-QuidelOrtho immunodiagnostic systems provides a robust, scalable method to enhance chemiluminescence signals across a variety of clinical and research settings (Vitros®-QuidelOrtho, Intellicheck® Technology). These results demonstrate the potential of the USE method to significantly improve the sensitivity and detection range of standard ELISA assays, which could have important implications for detecting low-abundance biomarkers in clinical and research applications.

### Example 15

### Schematic Representation of MIP Technology Used in Cell and Extracellular Vesicle Capture Applications

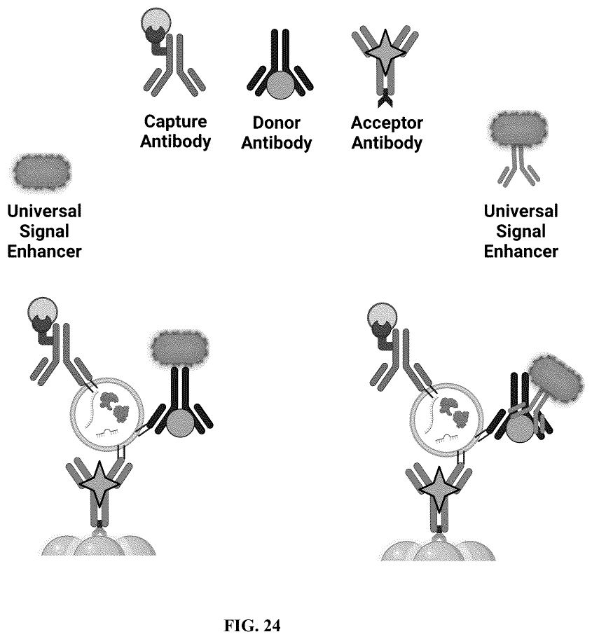

This example describes the application of Multi-Omic Integration Platform (MIP) technology for cell and extracellular vesicle capture, as illustrated in FIG. 24. The process begins with the isolation of cells or extracellular vesicles using a capture antibody that targets specific membrane proteins. These captured entities are then further functionalized with donor antibodies targeting another prevalent membrane protein. Magnetic beads are employed to extract the captured cells or vesicles. Following extraction, the separated cells or vesicles are transferred to a plate modified with acceptor antibodies on its surface. These acceptor antibodies target either the same or different membrane proteins to complete the formation of a BRET (Bioluminescent Resonance Energy Transfer) complex. The final detection step is carried out using either MIP assay based on Method 1 or Method 2.

FIG. 24 provides a schematic representation of this entire process, visually illustrating how the MIP technology can be adapted for cell and extracellular vesicle capture applications. This application of MIP technology demonstrates its versatility beyond molecular biomarker detection. By enabling the capture and analysis of entire cells or extracellular vesicles, this approach could provide more comprehensive insights into cellular states and intercellular communication. This could be particularly valuable in fields such as cancer research, where understanding the characteristics of circulating tumor cells or tumor-derived extracellular vesicles could lead to improved diagnostics or therapeutic strategies.

The ability to use either Method 1 or Method 2 for detection also highlights the flexibility of the MIP platform, allowing researchers to choose the most appropriate method for their specific experimental needs. This adaptability, combined with the sensitivity and specificity of BRET-based detection, positions the MIP technology as a powerful tool for advanced cellular and vesicular analysis.

### Example 16

### Construction of a Multi Omic Integration Platform (MIP)

This example describes the construction of a Multi Omic Integration Platform (MIP), detailing the preparation of various components and processes involved.

### Preparation of the Nanoparticle Array

Cleaning the 96-well Plate: Each well is filled with 50 μL of 10% RBS detergent solution and incubated at room temperature for 15 minutes. The detergent is aspirated using a vacuum system, and the plates are tapped on absorbent paper to remove residual detergent. Each well is thoroughly rinsed with deionized (DI) water followed by Milli-Q water. The plates are covered and centrifuged upside down at 1000 rpm for 30 seconds. To ensure complete drying, nitrogen gas is used, and the plates are placed in a vacuum oven at 60° C. for 20 minutes.

Well Surface Activation: After cooling to room temperature, each well receives 50 μL of a 1:1 HCl/MeOH solution, and the plates are incubated at room temperature for 30 minutes. The solution is aspirated, and the plates are tapped on absorbent paper to remove excess liquid. The wells are rinsed with DI water followed by Milli-Q water. Plates are covered, centrifuged upside down for 30 seconds at 1000 rpm, dried with nitrogen gas, and placed in a vacuum oven at 60° C. for 2 hours.

APTES Functionalization: After cooling to room temperature, a 30% APTES solution (2.25 mL APTES in 15 mL ethanol) is prepared, and 50 μL is added to each well. The plates are incubated at room temperature for 30 minutes. The solution is aspirated, and each well is washed with 100 μL ethanol, shaken at 600 rpm for 1 minute, and aspirated. This ethanol wash is repeated three times. After the final wash, the plates are tapped on absorbent paper, centrifuged upside down for 30 seconds at 1000 rpm, dried with nitrogen gas, and placed in a vacuum oven at 60° C. overnight.

Functionalization with PEG-thiolated Gold Nanospheres (AuSPs): Following the preparation of APTES-functionalized well plates, 50 μL of amine-PEG-thiolated gold nanospheres (AuSPs) with an optical density (OD) of 3 are added to each well. The plates are incubated at 100 rpm at room temperature for 2 hours on a plate shaker. Post-incubation, the wells are washed three times with Milli-Q water using a plate washer at 600 rpm to remove unbound nanoparticles. Attachment of the AuSPs is confirmed by a UV absorbance peak at ˜520 nm. Automated liquid handling systems (epMotion® 5075) and a plate washer (Biotek 405) ensure consistent pipetting, aspiration, and washing for uniform plate preparation and functionalization.

### Preparation and Functionalization of Acceptor Probes/Antibodies on AuSP-Functionalized Nanoparticle Arrays

Acceptor Probes (Method 1 and Method 2): The acceptor probes consist of single-stranded DNA (ssDNA) with a thiol group at the 3′ end and an Oregon Green (OG) fluorophore at the 5′ end (OG-Acceptor Probe-S—S). The sequences, 5-60 bases in length, are designed to complement either the target analyte or an extended probe and are custom-synthesized by IDT. The thiol-modified ssDNAs (in disulfide form) are reduced with Bond-Breaker TCEP solution (2 μL of 0.5 M TCEP to 100 μL of 100 M oligos) and incubated for 1 hour at room temperature on a rotary shaker, producing ssDNA-Acceptor Probe-SH. This solution is diluted to 10 μg/mL by adding 7.81 mL of 1×TE solution to 102 μL of the treated oligos, resulting in a total volume of ˜7.91 mL. The treated oligos (70 μL per well) are immediately incubated overnight at 4° C. on the AuSP-functionalized nanoparticle array well plates. After incubation, the plates are aspirated and blocked with SuperBlock buffer for 4 hours, followed by three washes with a washing buffer containing Tris (1×), 0.1% bovine serum albumin (BSA), and 0.05% Tween 20.

Acceptor Antibodies (for Method 1 and Method 2): Acceptor antibodies are labeled with Oregon Green using an Oregon Green halo tag linker (OG-HL) from Promega. Antibody buffer exchange is performed using a Zeba spin desalting column with 10 mM sodium bicarbonate (pH 8.5). 1 μL of OG-HL is added to 100 μg/mL of antibody and incubated for 90 minutes at room temperature. After incubation, 1% acetic anhydride is added, and the reaction proceeds for 2 hours at 4° C. to block unbound amines on the antibodies. A final buffer exchange into PBS is performed to remove free ligands, and the antibody concentration is measured via absorbance at 280 nm. Labeled antibodies are stored at 4° C. for short-term or in 50% glycerol at −20° C. for long-term storage.

Conjugation of OG-labeled Acceptor Antibodies with AuSPs: Fresh solutions of EDC (8 mg/mL) and Sulfo-NHS (22 mg/mL) are prepared in 100 μL of 0.1 M MES buffer (pH 4.7). 1 mL of OG-functionalized antibody (diluted to 10 μg/mL in 0.1 M PBS, pH 7.4) is mixed with 10 L of the EDC-Sulfo-NHS solution and incubated for 30 minutes at 37° C. 70 μL of the NHS-OG-Ab mixture is added to each well containing NH2-PEG-S-AuSPs and incubated overnight at 4° C. on a shaker (100 rpm). After incubation, excess EDC/Sulfo-NHS and unbound antibodies are aspirated, and each well is rinsed with 100 μL of conjugation buffer. To ensure removal of non-bound antibodies, wells are washed three times with washing buffer. 50 μL of Thermo Fisher SuperBlock is added for 30 minutes to block nonspecific sites, followed by a final wash with washing buffer.

### Preparation of Donor Probes and Donor Antibodies

Donor Probes (for Method 1): The donor probes are single-stranded DNA (ssDNA) conjugated with NanoLuc (nLuc) enzyme at the 3′ end to bind with the nLuc/HaloTag protein (HP). The probes, 22-25 bases long, are designed to complement either the target analyte or an extended probe, with sequences sourced from IDT, including a 3′ HaloTag linker. NanoLuc-HaloTag is conjugated to 3′-HL-ssDNA using a 4-fold molar excess of NanoLuc-HaloTag protein. The mixture is incubated with 10% IGEPAL® CA-630 (900 μL PBS+100 μL IGEPAL® CA-630) for 16-20 hours at 4° C. with gentle mixing. For purification, the solution is dissolved in 100 μL of deionized water and extracted three times with an equal volume of chloroform. NanoLuc-ssDNA is then precipitated by adding one-tenth volume of 3 M NaCl and 2.5 times the volume of cold absolute ethanol. The mixture is incubated at −20° C. for 30 minutes, centrifuged at 12,000×g for 30 minutes, and the supernatant removed. The pellet is rinsed with cold 70% ethanol, dried under vacuum, and redissolved in deionized water to 25 μg/mL. The conjugate is stored at 4° C. for short-term use or in 50% glycerol at −20° C. for long-term storage.

Donor Probes (for Method 2): In Method 2, the donor probe includes a 5′ biotin tag and a 3′ HaloTag. The complementary sequence with a 5′ biotin tag is sourced from IDT. The remaining protocol follows the same steps as Method 1.

Donor Antibodies (Method 1 and Method 2): Donor antibodies are labeled with NanoLuc® luciferase using a two-step process. First, antibodies are conjugated with the HaloTag ligand via an amine-reactive HaloTag® succinimidyl ester (04) ligand. The antibody buffer is exchanged into 10 mM sodium bicarbonate (pH 8.5) using a Zeba spin desalting column. 1 μL of HaloTag ligand is added to 100 μg/mL of antibody and incubated for 90 minutes at room temperature. After incubation, a buffer exchange into PBS is performed to remove free ligand, and the labeled antibody concentration is determined by absorbance at 280 nm. In the second step, NanoLuc-HaloTag is conjugated to the HaloTag-labeled antibody. A 4-molar excess of NanoLuc-HaloTag is added, and the mixture is incubated with 10% IGEPAL® CA-630 (900 μL PBS+100 L IGEPAL® CA-630) for 16-20 hours at 4° C. with gentle mixing. After incubation, buffer exchange into PBS is performed to remove free ligands, and the antibody concentration is again measured via absorbance at 280 nm. The conjugates are stored at 4° C. for short-term use or in 50% glycerol at −20° C. for long-term storage.

### Preparation of Capture Probes and Capture Antibodies

Capture Probes (Method 1): Capture probes consist of single-stranded DNA (ssDNA) with a photocleavable spacer and a biotin affinity tag at the 5′ end, referred to as capture probe-PC biotin. For small RNA applications, two probe types are used: Capture Probe 1 (10-20 bases) complementary to one half of the target RNA, and Capture Probe 2 (50-100 bases) with a sequence complementary to the remainder of the RNA and designed to hybridize with donor and acceptor probes. These sequences are synthesized by IDT. Streptavidin magnetic beads (Catalog 65001) are used for magnetic separation.

Capture Probes (Method 2): In Method 2, capture probe-PC—NH2 is used. COOH-Streptavidin magnetic beads (Catalog 65012) are employed for magnetic separation with a biotin tag on the donor probe.

Capture Antibodies (Method 1): For protein detection, primary monoclonal antibodies specific to the target antigen are biotinylated with a photocleavable spacer. Antibodies are functionalized with PC biotin-PEG3-NHS ester (Sigma Aldrich 901767) following buffer exchange using a Zeba spin desalting column with 10 mM sodium bicarbonate (pH 8.5). The antibody concentration is adjusted to 1 mg/mL, and a 10 mg/mL PC biotin-PEG3-NHS solution in DMSO is prepared. The biotin reagent is added to the antibody at a 5:1 ratio, and the mixture is incubated overnight at 4° C. Excess biotin is removed by buffer exchange into PBS. Antibody concentration is determined by measuring absorbance at 280 nm (Abs ˜1.4 corresponds to 1.0 mg/mL antibody).

Capture Antibodies (Method 2): In Method 2, additional steps are performed to conjugate PC biotin-PEG3-NHS-functionalized antibodies with HaloTag linker. PC biotin-PEG3-NHS-functionalized antibodies are then conjugated with HaloTag ligand using an amine-reactive HaloTag® succinimidyl ester (04). Buffer exchange is performed with 10 mM sodium bicarbonate (pH 8.5) using a Zeba desalting column. 1 μL of HaloTag ligand is added to 100 μg/mL of antibody and incubated for 90 minutes at room temperature. Excess ligand is removed via buffer exchange into PBS, and antibody concentration is measured by absorbance at 280 nm.

### Preparation of Universal Signal Enhancer Solution

For Method 1: HS-PEG-COOH functionalized gold nanorods (AuNRs) are used. Anti-NanoLuc antibodies (10 μg/mL, Promega) are paird to the AuNRs using the EDC/NHS protocol as described in the acceptor antibody section. The antibody mixture is added directly to the AuNR solution (1 OD) and incubated for 2 hours at room temperature. The anti-NanoLuc-functionalized AuNRs are purified using an Amicon® Ultra Centrifugal Filter (100 kDa MWCO, Millipore Sigma, UFC9100). The purified AuNRs are resuspended in 0.25 OD PBS buffer and stored at 4° C. for future use.

For Method 2: HS-PEG-COOH functionalized gold nanorods (AuNRs) are used. Streptavidin is paird to the AuNRs using the EDC/NHS protocol.

Although the invention has been described with reference to the above examples, it should be understood that modifications and variations are encompassed within the spirit and scope of the invention. Accordingly, the invention is limited only by the following claims.

What is claimed is:

1. A target analyte detection system comprising:
(a) a capture probe comprising an affinity tag and a cleavable linker or photo-cleavable spacer;
(b) a donor probe;
(c) an acceptor probe;
(d) a nanoparticle array comprising a substrate with nanoparticles, functionalized with the acceptor probes on the surface of the substrate and a spacer between the nanoparticle and the acceptor probe; and
(e) a universal signal enhancer solution comprising nanoparticles, wherein each nanoparticle is conjugated with one or more of: an anti-bioluminescent antibody for donor bioluminescent tag, an anti-fluorescent antibody for donor fluorescent tag, an anti-chemiluminescent antibody for donor chemiluminescent tag and an affinity tag,
wherein the donor probe and the acceptor probe comprise a BRET signal pair, a FRET signal pair, a bioluminescent subunit complex, a bioluminescent signal emitter, a fluorescent signal emitter, a chemiluminescent signal emitter, a LSPR signal, or an LSPR signal pair.

2. The detection system of claim 1, wherein the donor probe comprises an affinity tag and a bioluminescent tag, a first fluorescent tag, first bioluminescent subunit tag or a chemiluminescence tag.

3. The detection system of claim 1, wherein the acceptor probe comprises an affinity tag and one or more of: a fluorescent tag compatible with the bioluminescent tag, a second fluorescent tag compatible with the fluorescent tag, or a second bioluminescent subunit tag compatible with the first bioluminescent subunit tag.

4. The detection system of claim 1, wherein the detection mechanism includes one or more resonance energy transfer (RET) mechanisms, including but not limited to BRET, FRET, CRET, ECL, SPA, QRET, LSPR, BiFC, TR-FRET, fluorescence anisotropy, SPR, luminescence, chemiluminescence, Raman scattering, or FLIM, for detecting and measuring interactions between donor and acceptor molecules.

5. The detection system of claim 1, wherein the BRET signal pair, the FRET signal pair, the bioluminescent signal emitter, or the fluorescence signal emitter is located:
(a) between about 5 nm to 60 nm from nanoparticles in the nanoparticle array; or
(b) between about 5 nm to 60 nm from the nanoparticle of the universal signal enhancer solution.

6. The detection system of claim 1, wherein signal enhancement is achieved using Localized Surface Plasmon Resonance (LSPR) of the nanoparticle of the nanoparticle array and the nanoparticle of the universal signal enhancer solution.

7. The detection system of claim 1, wherein signal enhancement is achieved:
(a) using LSPR of the nanoparticle of the nanoparticle array only; or
(b) using LSPR of the nanoparticle of the universal signal enhancer solution only.

8. The detection system of claim 1, wherein analyte detection can be used without signal enhancement from the nanoparticle of the nanoparticle array or the nanoparticle of the universal signal enhancer solution.

9. The detection system of claim 1, wherein the target analyte is:
(a) a nucleic acid selected from DNA, lncRNA, mRNA, tRNA, rRNA;
(b) small RNA selected from the group consisting of short RNA, microRNA (miRNA), tiny non-coding RNAs (tncRNA), small modulatory RNA, small interfering RNAs (siRNAs), Piwi-interacting RNAs (piRNAs), transfer RNA-derived small RNAs (tsRNAs or tRFs), small nuclear RNAs (snRNAs), small nucleolar RNAs (snoRNAs), small RNA fragments derived from ribosomal RNAs (rRFs), small modulatory RNAs (smRNAs), small guide RNAs (sgRNAs), and small temporal RNAs (stRNAs); or (c) a protein.

10. The detection system of claim 9, further comprising an extended probe, wherein the extended probe comprises a sequence of 25-100 nucleotides, wherein about 6-20 nucleotides are complementary to a portion of the small RNA, and remaining bases of the extended probe are complementary to one or more of the donor probes and the acceptor probe.

11. The detection system of claim 1, wherein the capture probe comprises a nucleotide sequence about 10 to 100 or 25 to 50 nucleotides in length, wherein the capture probe is complementary to a target analyte, and wherein the capture probe comprises an affinity tag and a cleavable spacer attached to the 5′ end of the capture probe.

12. The detection system of claim 11, wherein the affinity tag comprises His-tag, FLAG-tag, Glutathione S-transferase (GST) tag, Maltose Binding Protein (MBP) tag, Strep-tag, HA-tag, Myc-tag, Avi-tag, V5-tag, T7-tag, biotin or its derivatives, SNAP tag, CLIP tag, or HaloTag®, and wherein the affinity tag comprises amine, carboxylic acid, amino acid, thiol (SH), hydroxyl (OH), phosphate, azide group, alkaline group, ketone group, biotin or a functional derivative thereof, or a halide group, or biotin or a functional derivative thereof.

13. The detection system of claim 1, wherein the donor probe comprises a nucleotide sequence of about 10-100 nucleotides or about 15 to 30 nucleotides in length, wherein the donor probe is complementary to the target analyte or the extended probe, and wherein the donor probe comprises a bioluminescent tag or a fluorophore tag or bioluminescent subunit tag or chemiluminescence tag attached to the 3′ end of the donor probe and an affinity tag attached to the 5′ end for binding with the affinity tag on the universal signal enhancer solution nanoparticles.

14. The detection system of claim 1, wherein the acceptor probe comprises a nucleotide sequence about 6 to 100 nucleotides or about 10 to 25 nucleotides in length, wherein the acceptor probe is complementary to the target analyte or the extended probe, wherein the acceptor probe comprises an affinity tag and one or more of: a fluorescent tag compatible with the bioluminescent tag, a second fluorescent tag compatible with the first fluorescent tag, a second bioluminescent subunit tag compatible with the first bioluminescent subunit tag or a fluorescent tag compatible chemiluminescence tag, wherein the acceptor probe comprises a spacer between the affinity tag and the nucleotide sequence, and wherein the spacer comprises hydrocarbon chain, polyethylene glycol (PEG), a polyamino acid, a polyacrylamide, polyvinylpyrrolidone, a zwitterionic polymer, a polysaccharide, poly(N-(2-hydroxypropyl)methacrylamide), poly(oligo(ethylene glycol), methylether methacrylate, a carboxylic dextran, a hydrocarbon chain, a substituted hydrocarbon chain, a silane group, 3-mercaptopropyl triethoxysilane (MPTES), hydrocarbon chain, wherein the spacer comprises a 1-20 nm distance from nanoparticles on the nanoparticle array.

15. The detection system of claim 14, wherein the acceptor probe is bound to the donor probe via analyte to create:
(a) a bioluminescent resonance energy transfer (BRET) complex;
(b) a fluorescence resonance energy transfer (FRET) complex;
(c) a bioluminescent complex;
(d) a bioluminescent signal;
(e) a fluorescence signal; or
(f) a chemiluminescence signal.

16. The detection system of claim 1, wherein the target analyte is a protein, and wherein the capture probe is a capture antibody, the donor probe is a donor antibody, the acceptor probe is an acceptor antibody.

17. The detection system of claim 16, wherein the capture antibody comprises a photocleavable spacer with a biotin label, and epitope binding sites for the target analyte.

18. The detection system of claim 17, further comprising an affinity tag to bind with the nanoparticle of the universal signal enhancer solution, wherein the affinity tag is located on either: (a) the capture antibody; or (b) the donor antibody.

19. The detection system of claim 18, wherein the affinity tag comprises a: His-tag, FLAG-tag, Glutathione S-transferase (GST) tag, Maltose Binding Protein (MBP) tag, Strep-tag, HA-tag, Myc-tag, Avi-tag, V5-tag, T7-tag, biotin, SNAP tag, CLIP tag, HaloTag®, amine, carboxylic acid, amino acid, thiol (SH), hydroxyl (OH), phosphate, azide group, alkaline group, ketone group, or a halide group, or a functional equivalent thereof.

20. The detection system of claim 16, wherein the donor antibody comprises a bioluminescent tag and epitope binding sites for the target analyte.

21. The detection system of claim 20, further comprising an affinity tag to bind with the nanoparticle of the universal signal enhancer solution, wherein the affinity tag is located on either: (a) the capture antibody; or (b) the donor antibody.

22. The detection system of claim 20, wherein the bioluminescent tag is Luciferin, Luciferyl adenylate firefly luciferase, Renilla luciferase, aequorin, Gaussia luciferase, or bacterial luciferase, coelenterazine aequorin, dinoflagellate luciferin Photoprotein, nanoluc luciferase, cypridina luciferase, nanobit-smallbit, nanobit-largebit or a luciferin or a functional equivalent thereof.

23. The detection system of claim 16, wherein the acceptor antibody comprises a fluorescent tag or a bioluminescent tag, an affinity tag, and binding sites for the donor antibody, wherein the donor antibody is complexed with the target analyte and the capture antibody.

24. The detection system of claim 23, wherein the acceptor antibody is bound to the donor antibody to create:
(a) a bioluminescent resonance energy transfer (BRET) complex;
(b) a fluorescence resonance energy transfer (FRET) complex;
(c) a bioluminescent complex;
(d) a bioluminescent signal;
(e) a fluorescence signal;
(f) a chemiluminescence signal; or
(g) a LSPR signal.

25. The detection system of claim 1, wherein the substrate comprises a custom nanofiber array, glass, an elastomeric polymer, polydimethylsiloxane (PDMS), ECOFLEX®, SILBIONE®, polyethylene terephthalate (PET), polyurethane (PU), polyethylene naphthalate (PEN), a polyimide (PI), polybutadiene, polyisoprene, a silane, a polyamine, polymethylmethacrylate (PMMA), polydopamine, polyvinylpyrrolidone (PVP), polyvinyl alcohol (PVA), a polyolefin, a polyamide, a polyimide, a protein, silk, cellulose, a polyelectrolyte, a peptoid, or a combination thereof.

26. The detection system of claim 1, wherein the nanoparticle comprises gold, silver, iron oxide, a quantum dot, a carbon-based nanoparticle, a chemical nanoparticle, a liposome, a polymeric nanoparticle, a dendrimer, a magnetic nanoparticle, a silica nanoparticle, a metal oxide nanoparticle, a semiconductor nanoparticle, an MXene nanoparticle, or a combination thereof.

27. The detection system of claim 1, wherein the nanoparticle is spherical, rod-shaped, triangular, prismatic, cube-shaped, star-shaped, wire-shaped, a sheet, tube-shaped, hollow or cage-like, flower-shaped, disk-shaped, a nanopyramid, a nanobipyramid, a nanoplate, a self-assembled nanostructure, a bowtie antenna, a nano island, a nanoshell, or a combination thereof.

28. The detection system of claim 1, wherein the nanoparticle is about 4 nm to about 2000 nm.

29. The detection system of claim 1, wherein the spacer between the nanoparticle and the acceptor probe has a thickness about 0.5 nm to about 20 nm, and wherein the spacer comprises polyethylene glycol (PEG), a polyamino acid, a polyacrylamide, polyvinylpyrrolidone, a zwitterionic polymer, a polysaccharide, poly(N-(2-hydroxypropyl)methacrylamide), poly(oligo(ethylene glycol), methylether methacrylate, a carboxylic dextran, a hydrocarbon chain, a substituted hydrocarbon chain, a silane group, 3-mercaptopropyl triethoxysilane (MPTES), or a combination thereof.

30. The detection system of claim 16, wherein the spacer between the nanoparticle and the acceptor antibody has a thickness about 0.5 nm to about 20 nm.

31. The detection system of claim 1, wherein the universal enhancer solution comprises nanoparticles, each nanoparticle being conjugated with one or more of the following:
(a) an affinity tag that binds to an affinity tag on the donor probe or the donor antibody or capture antibody;
(b) an anti-bioluminescent antibody that binds to a bioluminescent tag on the donor probe or the donor antibody;
(c) an anti-fluorescent antibody that binds to a fluorescent tag on the donor probe or the donor antibody; or
(d) an anti-chemiluminescent antibody that binds to a chemiluminescent tag on the donor probe or the donor antibody.

32. The detection system of claim 31, further comprising a spacer between the nanoparticle and the anti-fluorescent/bioluminescent/chemiluminescent antibody or affinity tag, wherein the spacer comprises polyethylene glycol (PEG), a polyamino acid, a polyacrylamide, polyvinylpyrrolidone, a zwitterionic polymer, a polysaccharide, poly(N-(2-hydroxypropyl)methacrylamide), poly(oligo(ethylene glycol), methylether methacrylate, a carboxylic dextran, a hydrocarbon chain, a substituted hydrocarbon chain, a silane group, 3-mercaptopropyl triethoxysilane (MPTES), or a combination thereof, and wherein the spacer has a thickness of about 0.5 nm to about 20 nm.

33. The detection system of claim 31, wherein the nanoparticle comprises gold, silver, iron oxide, a quantum dot, a carbon-based nanoparticle, a chemical nanoparticle, a liposome, a polymeric nanoparticle, a dendrimer, a magnetic nanoparticle, a silica nanoparticle, a metal oxide nanoparticle, a semiconductor nanoparticle, an MXene nanoparticle, or a combination thereof.

34. The detection system of claim 31, wherein the nanoparticle is spherical, rod-shaped, triangular, prismatic, cube-shaped, star-shaped, wire-shaped, a sheet, tube-shaped, hollow or cage-like, flower-shaped, disk-shaped, a nanopyramid, a nanobipyramid, a nanoplate, a self-assembled nanostructure, a bowtie antenna, a nano island, a nanoshell, or a combination thereof.

35. The detection system of claim 31, wherein the nanoparticle is about 4 nm to about 2000 nm, and wherein the thickness from the nanoparticle surface to the donor bioluminescent and fluorescence tag is about 0.5 nm to about 20 nm.

36. The detection system of claim 1, wherein the donor probe comprises a bioluminescent tag attached to the 3′ end to bind with the anti-bioluminescent antibody on the nanoparticle of the universal signal enhancer.

37. The detection system of claim 36, wherein the bioluminescent tag is Luciferin, Luciferyl adenylate firefly luciferase, Renilla luciferase, aequorin, Gaussia luciferase, or bacterial luciferase, coelenterazine aequorin, dinoflagellate luciferin Photoprotein, nanoluc luciferase, cypridina luciferase, nanobit-smallbit, nanobit-largebit or a functional equivalent thereof.

38. The detection system of claim 16, wherein the donor antibody comprises a bioluminescent tag to bind with the anti-bioluminescent antibody on the nanoparticle of the universal signal enhancer.

39. The detection system of claim 38, wherein the bioluminescent tag is Luciferin, Luciferyl adenylate firefly luciferase, Renilla luciferase, aequorin, Gaussia luciferase, or bacterial luciferase, coelenterazine aequorin, dinoflagellate luciferin Photoprotein, nanoluc luciferase, cypridina luciferase, nanobit-smallbit, nanobit-largebit or a functional equivalent thereof.

40. A method for detection of a target analyte, comprising the detection system of claim 1, wherein the method comprises:
(a) hybridizing the capture probe to the target analyte to form a capture probe-target analyte complex, wherein the capture probe comprises a photocleavable spacer with a biotin label affinity tag;
(b) attaching the biotin label to a magnetic bead;
(c) separating the capture probe-target analyte complex with a magnet;
(d) hybridizing the donor probe to the target analyte to form a capture probe-target analyte-donor probe complex, wherein the donor probe comprises an affinity tag and a bioluminescent tag;
(e) separating the capture probe-target analyte-donor probe complex with a magnet;
(f) exposing the capture probe-target analyte-donor probe complex to ultraviolet light to detach the magnetic bead from the complex;
(g) contacting the capture probe-target analyte-donor probe complex with acceptor probes on the nanoparticle array to create a BRET or FRET complex;
(h) adding the universal signal enhancer solution of claim 1; and
(i) introducing a substrate reagent to generate light emission.

41. The method of claim 40, further comprising hybridizing an extended probe to the target analyte and capture probe.

42. The detection system of claim 2, wherein the bioluminescent tag on the donor probe and the fluorescent tag on the acceptor probe are positioned at a distance less than 10 nm apart.

43. The detection system of claim 3, wherein the first fluorescent tag and the second fluorescent tag are positioned at a distance less than 10 nm apart.

44. The detection system of claim 3, wherein the first bioluminescent subunit tag and the second bioluminescent subunit tag are positioned at a distance less than 10 nm apart.

45. The detection system of claim 1, wherein the donor probe comprises no tag, and acceptor probe comprises no tag, and the detection system generates a localized surface plasmon resonance (LSPR) signal.

46. The detection system of claim 1, wherein the donor probe comprises an affinity tag for interaction with a universal signal enhancer, and the acceptor probe comprises no tag, and the detection system generates a localized surface plasmon resonance (LSPR) signal.

47. The detection system of claim 1, wherein the universal signal enhancer solution increases the sensitivity of chemiluminescence detection by at least 1 to 1,000 or more times.

48. The detection system of claim 47, wherein the universal signal enhancer solution containing one nanoparticle increases the sensitivity of standard chemiluminescence detection by at least about 10, 20, 30, 40, 50 or more times.
